(19) United States
(12) Patent Application Publication
(10) Pub. No.: US 2020/0094066 A1
(43) Pub. Date: Mar. 26, 2020

(54) SYSTEMS, APPARATUS, AND/OR METHODS UTILIZING LASER GENERATING PULSES OF LIGHT, PULSED SOUND FREQUENCIES AND/OR PULSED ELECTROMAGNETIC FIELDS (EMFs) FOR IMPROVING PROPER SYSTEMS, APPARATUS, AND/OR METHODS UTILIZING LASER GENERATING PULSES OF LIGHT, PULSED SOUND FREQUENCIES AND/OR PULSED ELECTROMAGNETIC FIELDS (EMFs) FOR IMPROVING PROPERTIES OR FUNCTIONS OF CELLS, TISSUES, PROTEINS, FATS, OR ORGANS IN VITRO, IN VIVO, OR IN SITU

(71) Applicant: Stephan Heath, Littleton, CO (US)

(72) Inventor: Stephan Heath, Littleton, CO (US)

(21) Appl. No.: 16/577,154

(22) Filed: Sep. 20, 2019

Related U.S. Application Data
(60) Provisional application No. 62/734,000, filed on Sep. 20, 2018.

(51) Int. Cl. A61N 2/00; A61N 5/06; A61N 7/00; A61N 2/02; A61L 27/36

(52) U.S. Cl. CPC A61N 2/002; A61N 5/0616; A61N 5/0624; A61N 7/00; A61N 2/02; A61N 2005/067; A61N 2/004; A61N 2005/0663; A61N 2005/066; A61N 2005/0659; A61N 2005/0661; A61L 27/3691

(57) ABSTRACT

Systems, apparatus, and methods utilizing laser generating pulses of light, pulsed sound frequencies and/or pulsed electromagnetic fields (EMFs) for improving properties or functions of cells, tissues, proteins, fats, or organs in vitro, in vivo, or in situ,

### FIELD OF THE INVENTION

The invention relates to one or more of systems, apparatus, and methods utilizing laser generating pulses of light. pulsed sound frequencies and/or pulsed electromagnetic fields (EMFs) for improving properties or functions of cells, tissues, proteins, fats, or organs in vitro, in vivo, or in situ.

### BACKGROUND

DNA damage is an abnormal chemical structure in DNA, while a mutation is a change in the sequence of standard base pairs. DNA damages cause changes in the structure of the genetic material and prevents the replication mechanism from functioning and performing properly. DNA damage and mutation have different biological consequences. While most DNA damages can undergo DNA repair, such repair is not 100 percent efficient. Un-repaired DNA damages accumulate in non-replicating cells, such as cells in the brains or muscles of adult mammals and can cause aging. In replicating cells, such as cells lining the colon, errors occur upon replication past damages in the template strand of DNA or during repair of DNA damages. These errors can give rise to mutations or epigenetic alterations. These alterations can change gene function or regulation of gene expression and possibly contribute to progression to cancer.

All cancers begin in cells. The human body is made up of more than a hundred million-million (100,000,000,000,000) cells. Cancer starts with changes in one cell or a small group of cells. Usually, the human body has the right number of each type of cell. This is because cells produce signals to control how much and how often the cells divide. If any of these signals are faulty or missing, cells may start to grow and multiply too much and form a lump called a tumor. For a cancer to start, certain changes take place within the genes of a cell or a group of cells. All cancer cells are abnormal cells that occurs when normal cell division are disrupted. Cancer develops from changes that cause normal cells to acquire abnormal functions. Sometimes a change happens in the genes when a cell divides. This is a mutation. It means that a gene has been damaged or lost or copied twice. Mutations can happen by chance when a cell is dividing. Some mutations in genes that regulate cell division, apoptosis (cell death), and DNA repair may result in uncontrolled cell proliferation and cancer.

There is a need to provide new technologies utilizing laser generating pulses of light, pulsed sound frequencies and pulsed electromagnetic fields (EMFs) for improving properties or functions of cells, tissues, proteins, fats, or organs in vitro, in vivo, or in situ.

### SUMMARY OF THE INVENTION

Alternative and/or optional embodiments of the invention relate to systems, apparatus, and methods for treatment of abnormal cells, mutation cells or cancer cells and pathogens; and cellular markers of aging by utilizing laser generating pulses of light, pulsed sound frequencies and pulsed electromagnetic fields (EMFs) for improving properties or functions of cells, tissues, proteins, fats, or organs in vitro, in vivo, or in situ utilizing at least one or more pulsed electromagnetic field (EMF) comprising one or more of laser generating pulses of light, pulsed sound frequencies and pulsed resonant frequencies of one or more EMFs. Such systems, apparatus, and/or methods can optionally include the application of EMFs that are optionally applied to the cells, tissues, proteins, fats, or organs in vitro, in vivo, or in situ using laser generating pulses of light, pulsed sound frequencies and pulsed electromagnetic fields (EMFs) to focus on one or more EMFs on an area for improving properties or functions of cells, tissues, proteins, fats, or organs in vitro, in vivo, or in situ. The application of one or more EMFs to one or more cells, tissues, proteins, fats, or organs can optionally be done in vivo, ex vivo, and/or in vitro, to change a desired property or function of the one or more cells, tissues, proteins, fats, or organs in vitro, in vivo, or in situ. The changed property can optionally be selected from one or more of chemical, physical, or energetic properties. The function can include one or more chemical, physical, and/or energetic functions.

Conditions in the body are controlled, to provide a constant internal environment called homeostasis, The conditions that must he controlled include body temperature, water content, carbon dioxide level, and blood sugar level. Hormones are chemicals secreted by glands. Optionally the raising or lowering of one or more properties or functions of one or more cells, tissues, proteins, fats, or organs changes one or more conditions of one or more cells, tissues, proteins, this, or organs towards homeostasis or normal functioning of one or more cells, tissues, proteins, fats, or organs in vitro, in vivo, or in situ.

Non-limiting examples of properties include, but are not limited to, raising or lower 0.0001 to 10,000 percent of the concentration, amount, or volume (weight, mass, weight per volume, mass per volume, weight per weight, mass per weight, weight percent (wt. percent), volume, volume percent, mass per volume, moles, mole percent, moles per volume, moles per mass, moles per weight, and the like) of elements, molecules, and/or ions, e.g. optionally including one or more of carbon dioxide (CO2), oxygen (O2), hydroxide ions (OH−), hydrogen ions (H+), (e.g., pH), carbonate, biocarbonate, inflammation, magnesium, calcium, phosphate, potassium, chlorine, chloride, manganese, selenium, aluminum, mercury, monatomic gold, molecular geometry, sacred geometry, atomic geometry, amino acid(s), protein, saturated fatty acids, unsaturated fatty acids, sugars, triglycerides, ATPs, AMPs, ADPs, DNAs, RNAs, nucleic acids, ribonucleic acids, magnetic field, electric field, electromagnetic field, or the presence of non-naturally occurring compounds or compositions, such as, but not limited to, organic chemicals, pesticides, herbicides, glyphosate, DDT, DNA damage or mutation in cells, tissues, proteins, fats, or organs of diseased cells, tissues, proteins, fats, or organs of DNA damaged cells, tissues, proteins, fats, or organs of abnormal cells, mutation cells or cancer cells, and pathogens; and cellular markers of aging.

Optionally the raising or lowering of one or more properties or functions of one or more cells, tissues, proteins, fats, or organs changes one or more conditions of one or more cells, tissues, proteins, fats, or organs towards homeostasis or normal functioning of one or more cells, tissues, proteins, fats, or organs in vitro, in vivo, or in situ.

Non-limiting examples of functions of cells, tissues, proteins, fats, or organs include, but are not limited to, raising or lower 0.0001 to 10,000 percent of calories metabolized or burned, water uptake, homeostasis, plasma, lymph, urine, cytoplasmic, tissue, cell, organ, cerebrospinal fluid (CSF), saliva, nasal secretion, subcutaneous, intramuscular, or blood: pH, O2, CO2, carbonate, biocarbonate, glucose metabolism, fat metabolism, toxin excretion or removal, heavy metal excretion or removal, ostomosis, intracellular or extracellular transport, exocytosis, or cellular uptake.

The one or more frequencies optionally are selected from 1, 2, 3, 4, 5, b, 7, 8, 9, 10, 11, 12, 13, 14, or any range or value therein or between, optionally comprising one or more of laser generating pulses of light or more of; (a) one or more frequencies between 0.25 Hz and 11780000 Hz, optionally including (b) particular combinations of pulsed resonant frequencies, electromagnetic pulses, and/or one or more harmonics thereof; and applying such particular EMF frequency combinations onto cells, tissues, proteins, fats, or organs a particular area of the body for application to cells, tissues, proteins, fats, or organs of abnormal cells, mutation cells or cancer cells and pathogens with one or more pulsed electromagnetic fields (EMFs) of one or more pulsed resonant frequencies to provide EMF treated cells, tissues, proteins, fats, or organs in vitro, in vivo, or in situ.

Optionally the method includes wherein the application step (a) and/or the applying step (b) comprises the use of a generator or system of generators of pulsed resonant frequencies using copper rings to produce the one or more EMF at pulsed resonant frequencies.

Optionally the method includes wherein the use of laser generating pulses of light, pulsed sound frequencies and pulsed electromagnetic fields (EMFs) at pulsed frequencies using a frequency tone generator or system of frequency tone generators using one or more copper rings are provided as one or more copper rings with substantially the same radius, wherein the one or more copper rings intersect with each other such that the center of each disk is adjacent to a perimeter portion of the other copper rings that produce electromagnetic fields (EMFs) for application to cells, tissues, proteins, fats, or organs for improving properties or functions of cells, tissues, proteins, fats, or organs in vitro, in vivo, or in situ.

Optionally the method includes wherein the use of laser generating pulses of light, pulsed sound frequencies and pulsed electromagnetic fields (EMFs) at pulsed frequencies using a frequency tone generator or system of frequency tone generators using one or more copper rings vibrate at said at least one or more pulsed electromagnetic frequencies corresponding to the length, or a fraction or multiple of the length, of at least a portion of the copper rings that produce electromagnetic fields (EMFs) for application to cells, tissues, proteins, fats, or organs for improving properties or functions of cells, tissues, proteins, fats, or organs in vitro, in vivo, or in situ.

Optionally the method includes wherein the use of laser generating pulses of light, pulsed sound frequencies and pulsed electromagnetic fields (EMFs) at pulsed frequencies using a frequency tone generator or system of frequency tone generators wherein a primary coil of the copper or other metal rings creates at said one or more EMF frequencies that are resonant with a secondary frequency at which a secondary coil of the copper rings vibrate that balances the force of gravity within a magnetic field produced by the copper rings that produce electromagnetic fields (EMFs) for application to cells, tissues, proteins, fats, or organs for improving properties or functions of cells, tissues, proteins, fats, or organs in vitro, in vivo, or in situ.

The method or apparatus can optionally include utilizing a frequency tone generator or system of frequency generators, laser generating pulses of light, pulsed sound frequencies and EM fields alternating in polarity, either permanently or periodically, and magnetically charged copper rings in the shape of the vesica piscis, which creates a vortex of magnetic energy, that is the intersection of one or more copper rings with the same radius, intersecting in such a way that the center of each disk lies on the perimeter of the other. The positively charged end of the metal strand can optionally include the use of silver-soldered to the negatively charged end, bringing the copper wire to a neutral state. The copper ring creates a vortex of magnetic energy, a wavelength of invisible light that shines out both sides. The copper rings will vibrate at a pulsed, frequency based upon the length of a coiled copper wire. The copper rings are two pieces of twisted wires that are plated with precious metals. First, the copper or metal rings or electrodes are coated with a layer of silver and then a layer of 24 karat gold. This process is repeated with a layer of silver and layer of 24 karat gold for a total of nine layers. The primary and secondary circuits are tuned so they resonate at the same resonant frequency. This allows them to exchange energy, so the oscillating current alternates back and forth between the primary and secondary copper rings. The primary coil of the copper rings shall create an electrical circuit that consists of using electromagnetic fields (EMFs) alternating current electricity in combination with the energy field at a pulsed frequency compatible with the resonant frequency at which the secondary copper ring wants to vibrate that will balance the force of gravity to a pointe of neutralization within the magnetic fields. The frequencies and electromagnetic pulses pass through the copper coils to charge the cells. This electrical charge changes the frequency and/or behavior of the cells, tissues or organs in vitro, in vivo, or in situ.

Alternative and/or optional embodiments of the invention relate to systems, apparatus, and methods for application to non-healthy, abnormal or diseased cells, tissues or organs in the body utilizing at least one or more pulsed electromagnetic field (EMF) comprising one or more of laser generating pulses of light or more pulsed resonant frequencies the EMFs optionally applied to the cells, tissues or organs utilizing laser generating pulses of light at pulsed resonant frequencies with one or more pulsed electromagnetic fields (EMFs) of one or more pulsed resonant frequencies to provide EMF treated cells, tissues, proteins, fats, or organs in vitro, in vivo. or in situ.

Alternative and/or optional embodiments of the invention relate to systems, apparatus, and methods for application to diseased cells, tissues or organs in the heart using laser generating pulses of light, pulsed sound frequencies and at least one or more pulsed electromagnetic field (EMF) comprising one or more of laser generating pulses of light or more pulsed resonant frequencies the EMFs optionally applied to the cells, tissues or organs in the heart utilizing laser generating pulses of light to focus the application on cells, tissues or organs in the heart with one or more pulsed electromagnetic fields (EMFs) of one or more pulsed resonant frequencies to provide EMF treated cells, tissues, proteins, fats, or organs in the heart.

Alternative and/or optional embodiments of the invention relate to systems, apparatus, and methods for application to diseased cells, tissues or organs in the lungs utilizing at least one or more pulsed electromagnetic field (EMF) comprising one or more of laser generating pulses of light or more pulsed resonant frequencies the EMFs optionally applied to the cells, tissues or organs in the lungs utilizing laser generating pulses of light to focus the application on cells, tissues or organs in the lungs with one or more pulsed electromagnetic fields (EMFs) of one or more pulsed resonant frequencies to provide EMF treated cells, tissues, proteins, fats, or organs in the lungs.

Alternative and/or optional embodiments of the invention relate to systems, apparatus, and methods for application to diseased cells, tissues or organs in the adrenals utilizing at least one or more pulsed electromagnetic field (EMF) comprising one or more of laser generating pulses of light or more pulsed resonant frequencies the EMFs optionally applied to the cells, tissues or organs in the adrenals utilizing laser generating pulses of light to focus the application on cells, tissues or organs in the adrenals with one or more pulsed electromagnetic fields (EMFs) of one or more pulsed resonant frequencies to provide EMF treated cells, tissues, proteins, fats, or organs in the adrenals.

Alternative and/or optional embodiments of the invention relate to systems, apparatus, and methods for application to diseased cells, tissues or organs in the stomach utilizing at least one or more pulsed electromagnetic field (EMF) comprising one or more of laser generating pulses of light or more pulsed resonant frequencies the EMFs optionally applied to the cells, tissues or organs in the stomach utilizing laser generating pulses of light to focus the application on cells, tissues or organs in the stomach with one or more pulsed electromagnetic fields (EMFs) of one or more pulsed resonant frequencies to provide EMF treated cells, tissues, proteins, fats, or organs in the stomach.

Alternative and/or optional embodiments of the invention relate to systems, apparatus, and methods for application to diseased cells, tissues or organs in the kidney utilizing at least one or more pulsed electromagnetic field (EMF) comprising one or more of laser generating pulses of light or more pulsed resonant frequencies the EMFs optionally applied to the cells, tissues or organs in the kidney utilizing laser generating pulses of light to focus the application on cells, tissues or organs in the kidney with one or more pulsed electromagnetic fields (EMFs) of one or more pulsed resonant frequencies to provide EMF treated cells, tissues, proteins, fats, or organs in the kidney.

Alternative and/or optional embodiments of the invention relate to systems, apparatus, and methods for application to diseased cells, tissues or organs in the diaphragm utilizing at least one or more pulsed electromagnetic field (EMF) comprising one or more of laser generating pulses of light or more pulsed resonant frequencies the EMFs optionally applied to the cells, tissues or organs in the diaphragm utilizing laser generating pulses of light to focus the application on cells, tissues or organs in the diaphragm with one or more pulsed electromagnetic fields (EMFs) of one or more pulsed resonant frequencies to provide EMF treated cells, tissues, proteins, fats, or organs in the diaphragm.

Alternative and/or optional embodiments of the invention relate to systems, apparatus, and methods for application to diseased cells, tissues or organs in the spleen utilizing at least one or more pulsed electromagnetic field (EMF) comprising one or more of laser generating pulses of light or more pulsed resonant frequencies the EMFs optionally applied to the cells, tissues or organs in the spleen utilizing laser generating pulses of light to focus the application on cells, tissues or organs in the spleen with one or more pulsed electromagnetic fields (EMFs) of one or more pulsed resonant frequencies to provide EMF treated cells, tissues, proteins, fats, or organs in the spleen.

Alternative and/or optional embodiments of the invention relate to systems, apparatus, and methods for application to diseased cells, tissues or organs in the liver utilizing at least one or more pulsed electromagnetic field (EMF) comprising one or more of laser generating pulses of light or more pulsed resonant frequencies the EMFs optionally applied to the cells, tissues or organs in the liver utilizing laser generating pulses of light to focus the application on cells, tissues or organs in the liver with one or more pulsed electromagnetic fields (EMFs) of one or more pulsed resonant frequencies to provide EMF treated cells, tissues, proteins, fats, or organs in the liver.

Alternative and/or optional embodiments of the invention relate to systems, apparatus, and methods for application to diseased cells, tissues or organs in the pancreas utilizing at least one or more pulsed electromagnetic field (EMF) comprising one or more of laser generating pulses of light or more pulsed resonant frequencies the EMFs optionally applied to the cells, tissues or organs in the pancreas utilizing laser generating pulses of light to focus the application on cells, tissues or organs in the pancreas with one or more pulsed electromagnetic fields (EMFs) of one or more pulsed resonant frequencies to provide EMF treated cells, tissues, proteins, fats, or organs in the pancreas.

Alternative and/or optional embodiments of the invention relate to systems, apparatus, and methods for application to diseased cells, tissues or organs in the intestines utilizing at least one or more pulsed electromagnetic field (EMF) comprising one or more of laser generating pulses of light or more pulsed resonant frequencies the EMFs optionally applied to the cells, tissues or organs in the intestines utilizing laser generating pulses of light to focus the application on cells, tissues or organs in the intestines with one or more pulsed electromagnetic fields (EMFs) of one or more pulsed resonant frequencies to provide EMF treated cells, tissues, proteins, fats, or organs in the intestines.

Alternative and/or optional embodiments of the invention relate to systems, apparatus, and methods for application to diseased cells, tissues or organs in the gall bladder utilizing at least one or more pulsed electromagnetic field (EMF) comprising one or more of laser generating pulses of light or more pulsed resonant frequencies the EMFs optionally applied to the cells, tissues or organs in the gall bladder utilizing laser generating pulses of light to focus the application on cells, tissues or organs in the gall bladder with one or more pulsed electromagnetic fields (EMFs) of one or more pulsed resonant frequencies to provide EMF treated cells, tissues, proteins, fats, or organs in the gall bladder.

Alternative and/or optional embodiments of the invention relate to systems, apparatus, and methods for application to diseased cells, tissues or organs in the bladder utilizing at least one or more pulsed electromagnetic field (EMF) comprising one or more of laser generating pulses of light or more pulsed resonant frequencies the EMFs optionally applied to the cells, tissues or organs in the bladder utilizing laser generating pulses of light to focus the application on cells, tissues or organs in the bladder with one or more pulsed electromagnetic fields (EMFs) of one or more pulsed resonant frequencies to provide EMF treated cells, tissues, proteins, fats, or organs in the bladder.

Alternative and/or optional embodiments of the invention relate to systems, apparatus, and methods for application to diseased cells, tissues or organs in the fat cells utilizing at least one or more pulsed electromagnetic field (EMF) comprising one or more of laser generating pulses of light or more pulsed resonant frequencies the EMFs optionally applied to the cells, tissues or organs in the fat cells utilizing laser generating pulses of light to focus the application on cells, tissues or organs in the fat cells with one or more pulsed electromagnetic fields (EMFs) of one or more pulsed resonant frequencies to provide EMF treated cells, tissues, proteins, fats, or organs in the fat cells.

Alternative and/or optional embodiments of the invention relate to systems, apparatus, and. methods for application to diseased cells, tissues or organs in the muscles utilizing at least one or more pulsed electromagnetic field (EMF) comprising one or more of laser generating pulses of light or more pulsed resonant frequencies the EMFs optionally applied to the cells, tissues or organs in the muscles utilizing laser generating pulses of light to focus the application on cells, tissues or organs in the muscles with one or more pulsed electromagnetic fields (EMFs) of one or more pulsed resonant frequencies to provide EMF treated cells, tissues, proteins, fats, or organs in the muscles.

Alternative and/or optional embodiments of the invention relate to systems, apparatus, and methods for application to diseased cells, tissues or organs in the bone utilizing at least one or more pulsed electromagnetic field (EMF) comprising one or more of laser generating pulses of light or more pulsed resonant frequencies the EMFs optionally applied to the cells, tissues or organs in the bone utilizing laser generating pulses of light to focus the application on cells, tissues or organs in the cells, tissues, proteins, fats, or organs in the bone with one or more pulsed electromagnetic fields (EMFs) of one or more pulsed resonant frequencies to provide EMF treated cells, tissues, proteins, fats, or organs in the bone.

Alternative and/or optional embodiments of the invention relate to systems, apparatus, and methods for application to diseased cells, tissues or organs in the brain utilizing at least one or more pulsed electromagnetic field (EMF) comprising one or more of laser generating pulses of light or more pulsed resonant frequencies the EMFs optionally applied to the cells, tissues or organs in the brain utilizing laser generating pulses of light to focus the application on cells, tissues or organs in the brain with one or more pulsed electromagnetic fields (EMFs) of one or more pulsed resonant frequencies to provide EMF treated cells, tissues, proteins, fats, or organs in the brain.

Alternative and/or optional embodiments of the invention relate to systems, apparatus, and methods for application to diseased cells, tissues or organs in the blood utilizing at least one or more pulsed electromagnetic field (EMF) comprising one or more of laser generating pulses of light or more pulsed resonant frequencies the EMFs optionally applied to the cells, tissues or organs in the blood utilizing laser generating pulses of light to focus the application on cells, tissues or organs in the blood with one or more pulsed electromagnetic fields (EMFs) of one or more pulsed resonant frequencies to provide EMF treated cells, tissues, proteins, fats, or organs in the blood.

Alternative and/or optional embodiments of the invention relate to systems, apparatus, and methods for application to diseased cells, tissues or organs are selected wherein the increase, reduction, or improvement of the sugar levels in the blood is to an amount or concentration closer to normal physiological levels in the blood utilizing at least one or more pulsed electromagnetic field (EMF) comprising one or more of laser generating pulses of light or more pulsed resonant. frequencies the EMFs optionally applied to the cells, tissues or organs in the blood utilizing laser generating pulses of light to focus the application on cells, tissues or organs in the blood with one or more pulsed electromagnetic fields (EMFs) of one or more pulsed resonant frequencies to provide EMF treated cells, tissues, proteins, fats, or organs are selected wherein the increase, reduction, or improvement of the sugar levels in the blood is to an amount or concentration closer to normal physiological levels in the blood.

Alternative and/or optional embodiments of the invention relate to systems, apparatus, and methods for application to diseased cells, tissues or organs in the colon utilizing at least one or more pulsed electromagnetic field (EMF) comprising one or more of laser generating pulses of light or more pulsed resonant frequencies the EMFs optionally applied to the cells, tissues or organs in the colon utilizing laser generating pulses of light to focus the application on cells, tissues or organs in the colon with one or more pulsed electromagnetic fields (EMFs) of one or more pulsed resonant frequencies to provide EMF treated cells, tissues, proteins, fats, or organs in the colon.

Alternatively the raising or lowering of one or more properties or functions of one or more cells, tissues, proteins, fats, or organs changes one or more conditions of one or more cells, tissues, proteins, fats, or organs towards homeostasis or normal functioning of one or more cells, tissues, proteins, fats, or organs where non-homeostasis and/or non-normal or non-healthy cells can include the improved, properties or function of cells, tissues, proteins, fats, or organs is due to removal or inactivation of one or more of a non-naturally occurring substance with one or more pulsed electromagnetic fields (EMFs) of one or more pulsed resonant frequencies to provide EMF treated cells, tissues, proteins, fats, or organs in vitro, in vivo, or in situ.

Alternatively the raising or lowering of one or more properties or functions of one or more cells, tissues, proteins, fats, or organs changes one or more conditions of one or more cells, tissues, proteins, fats, or organs towards homeostasis or normal functioning of one or more cells, tissues, proteins, fats, or organs where non-homeostasis and/or non-normal or non-healthy cells can include the improved properties or function of cells, tissues, proteins, fats, or organs is due to removal or inactivation of one or more of abnormal cells, mutation cells or cancer cells with one or more pulsed electromagnetic fields (EMFs) of one or more pulsed resonant frequencies to provide EMF treated cells, tissues, proteins, fats, or organs in vitro, in vivo, or in situ.

Alternatively the raising or lowering of one or more properties or functions of one or more cells, tissues, proteins, fats, or organs changes one or more conditions of one or more cells, tissues, proteins, fats, or organs towards homeostasis or normal functioning of one or more cells, tissues, proteins, fats, or organs where non-homeostasis and/or non-normal or non-healthy cells can include the improved properties or function of cells, tissues, proteins, fats, or organs is due to DNA damage of abnormal chemical structure in DNA molecules with one or more pulsed electromagnetic fields (EMFs) of one or more pulsed resonant frequencies to provide EMF treated cells, tissues, proteins, fats, or organs in vitro, in vivo, or in situ.

Alternatively the raising or lowering of one or more properties or functions of one or more cells, tissues, proteins, fats, or organs changes one or more conditions of one or more cells, tissues, proteins, fats, or organs towards homeostasis or normal functioning of one or more cells, tissues, proteins, fats, or organs where non-homeostasis and/or non-normal or non-healthy cells can include the improved properties or function of cells, tissues, proteins, fats, or organs is due to DNA repair of cells with one or more pulsed electromagnetic fields (EMFs) of one or more pulsed resonant frequencies to provide EMF treated cells, tissues, proteins, fats, or organs in vitro, in vivo, or in situ.

Alternatively the raising or lowering of one or more properties or functions of one or more cells, tissues, proteins, fats, or organs changes one or more conditions of one or more cells, tissues, proteins, fats, or organs towards homeostasis or normal functioning of one or more cells, tissues, proteins, fats, or organs where non-homeostasis and/or non-normal or non-healthy cells can include the improved properties or function of cells, tissues, proteins, fats, or organs is due to DNA damage of abnormal chemical structure in DNA molecules to effect the cellular markers of aging in the human body with one or more pulsed electromagnetic fields (EMFs) of one or more pulsed resonant frequencies to provide EMF treated cells, tissues, proteins, fats, or organs in vitro, in vivo, or in situ.

### DETAILED DESCRIPTION

Alternative and/or optional embodiments of the invention relate to systems, apparatus, and methods for application to cells, tissues, proteins, fats, or organs utilizing at least one or more pulsed electromagnetic field (EMF) comprising one or more of laser generating pulses of light or more pulsed resonant frequencies of one or more EMFs. Such systems, apparatus, and/or methods can optionally include the application of EMFs that are optionally applied to the cells, tissues, proteins, fats, or organs in vitro, in vivo, or in situ utilizing laser generating pulses of light, pulsed sound frequencies and/or frequency tone generator or system of frequency tone generators to focus the one or more EMFs on an area for improving properties or functions of cells, tissues, proteins, fats, or organs in vitro, in vivo, or in situ. The application of one or more EMFs to one or more cells, tissues, proteins, fats, or organs can optionally be done in vivo, ex vivo, and/or in vitro, to change a desired property or function of the one or more cells, tissues, proteins, fats, or organs in vitro, in vivo, or in situ. The changed property can optionally be selected from one or more of chemical, physical, or energetic properties. The function can include one or more chemical, physical, and/or energetic functions.

Conditions in the body are controlled, to provide a constant internal environment called homeostasis. The conditions that must be controlled include body temperature, water content, carbon dioxide level, and blood sugar level. Hormones are chemicals secreted by glands. Optionally the raising or lowering of one or more properties or functions of one or more cells, tissues, proteins, fats, or organs changes one or more conditions of one or more cells, tissues, proteins, fats, or organs towards homeostasis or normal functioning of one or more cells, tissues, proteins, fats, or organs in vitro, in vivo, or in situ.

Optionally the raising or lowering of one or more properties or functions of one or more cells, tissues, proteins, fats, or organs changes one or more conditions of one or more cells, tissues, proteins, fats, or organs towards homeostasis or normal functioning of one or more cells, tissues, proteins, fats, or organs in vitro, in vivo, or in situ.

### DEFINITIONS

Absorption of electromagnetic radiation is the way in which the energy of a photon is taken up by matter, typically the electrons of an atom. Thus, the electromagnetic energy is transformed into internal energy of the absorber, for example thermal energy. The reduction in intensity of a light wave propagating through a medium by absorption of a part of its photons is often called attenuation. Usually, the absorption of waves does not depend on their intensity (linear absorption), although in certain conditions (usually, in optics), the medium changes its transparency dependently on the intensity of waves going through, and saturable absorption (or nonlinear absorption) occurs.

Acoustic resonance is a phenomenon where acoustic systems amplify sound frequencies whose frequency matches one of its own natural frequencies of vibration (its sound frequencies). The term “acoustic resonance” is sometimes used to narrow mechanical resonance to the frequency range of human hearing, but since acoustics is defined in general terms concerning vibrational waves in matter, acoustic resonance can occur at frequencies outside the range of human hearing. An acoustically resonant object usually has more than one resonance frequency, especially at harmonics of the strongest resonance. It will easily vibrate at those frequencies in Hz, and vibrate less strongly at other frequencies.

Aging is the process of becoming older. The term refers especially to human beings, many animals, and fungi, whereas for example bacteria, perennial plants and some simple animals are potentially immortal. The causes of aging are uncertain; current theories are assigned to the damage concept, whereby the accumulation of damage (such as DNA oxidation) may cause biological systems to fail, or to the programmed aging concept, whereby internal processes (such as DNA methylation) may cause aging. Programmed aging should not be confused with programmed cell death (apoptosis).

Amplitude of a wave is the distance from the center line (or the still position) to the top of a crest or to the bottom of a trough. Be careful with this quantity—the center line is not always given in a diagram. Amplitude is measured in meters (m). The greater the amplitude of a wave then the more energy it is carrying. The wavelength, λ, of a wave is the distance from any point on one wave to the same point on the next wave along. (The symbol is a Greek letter, ‘lambda’.) To avoid confusion, it is best to measure wavelength from the top of a crest to the top of the next crest, or from the bottom of a trough to the bottom of the next trough. Wavelength is also measured in meters (m)—it is a length after all. The frequency, f, of a wave is the number of waves passing a point in a certain time. We normally use a time of one second, so this gives frequency the unit hertz (Hz), since one hertz is equal to one wave per second. For water waves and sound frequencies the unit hertz is usually good enough but radio and TV waves have such a high frequency that the kilohertz (kHz) or even the megahertz (MHz) are better units. 1 kHz1000 Hz; 1 MHz=1000000 Hz. The speed (or sometimes you might see it called velocity) of a wave, v, is how far the wave travels in a certain time. Wave speed is measured in meters per second (m/).

Audio frequency (abbreviation: AF) or audible frequency is characterized as a periodic vibration whose frequency is audible to the average human. The SI unit of audio frequency is the hertz (Hz). It is the property of sound that most determines pitch. The generally accepted standard range of audible frequencies is 20 to 20,000 Hz, although the range of frequencies individuals hear is greatly influenced by environmental factors. Frequencies below 20 Hz are generally felt rather than heard, assuming the amplitude of the vibration is great enough. Frequencies above 20000 Hz can sometimes be sensed by young people. High frequencies are the first to be affected by hearing loss due to age and/or prolonged exposure to very loud noises.

Amplitude modulation (AM) is a modulation technique used in electronic communication, most commonly for transmitting information via a radio carrier wave. In amplitude modulation, the amplitude (signal strength) of the carrier wave is varied in proportion to that of the message signal being transmitted. The message signal is, for example, a function of the sound to be reproduced by a loudspeaker, or the light intensity of pixels of a television screen. This technique contrasts with frequency modulation, in which the frequency of the carrier signal is varied, and phase modulation, in which its phase is varied.

Bioelectrodynamics is a branch of medical physics and bioelectromagnetism which deals with rapidly changing electric and magnetic fields in biological systems, i.e. high frequency endogenous electromagnetic phenomena in living cells. Unlike the events studied by the electrophysiology, the generating mechanism of bioelectrodynamic phenomenon is not connected with currents of ions and its frequency is typically much higher. Examples include vibrations of electrically polar intracellular structures and non-thermal emission of photons as a result of metabolic activity.

Biomagnetism is the phenomenon of magnetic fields produced by living organisms; it is a subset of bioelectromagnetism. In contrast, organisms' use of magnetism in navigation is magnetoception and the study of the magnetic fields' effects on organisms is magnetobiology. (The word biomagnetism has also been used loosely to include magnetobiology, further encompassing almost any combination of the words magnetism, cosmology, and biology, such as “magnetoastrobiology”).

Biomarkers of aging are biomarkers that could predict functional capacity at some later age better than will chronological age. Stated another way, biomarkers of aging would give the true “biological age,” which may be different from the chronological age. Validated biomarkers of aging would allow for testing interventions to extend lifespan, because changes in the biomarkers would be observable throughout the lifespan of the organism. Although maximum lifespan would be a means of validating biomarkers of aging, it would not be a practical means for long-lived species such as humans because longitudinal studies would take far too much time. Ideally, biomarkers of aging should assay the biological process of aging and not a predisposition to disease, should cause a minimal amount of trauma to assay in the organism, and should be reproducibly measurable during a short interval compared to the lifespan of the organism. Although graying of hair increases with age, hair graying cannot be called a biomarker of aging. Similarly, skin wrinkles and other common changes seen with aging are not better indicators of future functionality than chronological age.

Body capacitance is the physical property of the human body that has it act as a capacitor. Like any other electrically-conductive object, a human body can store electric charge if insulated. The actual amount of capacitance varies with the surroundings; it would be low when standing on top of a pole with nothing nearby, but high when leaning against an insulated, but grounded large metal surface, such as a household refrigerator, or a metal wall in a factory. Synthetic fabrics and friction can charge a human body to about 3 kV. Low potentials may not have any notable effect, but some electronic devices can be damaged by modest voltages of 100 volts.

Cancer bacteria are bacteria infectious organisms that are known or suspected to cause cancer. While cancer-associated bacteria have long been considered to be opportunistic (i.e., infecting healthy tissues after cancer has already established itself), there is some evidence that bacteria may be directly carcinogenic. The strongest evidence to date involves the bacterium H. pylori and its role in gastric cancer. Oncoviruses are viral agents that are similarly suspected. of causing cancer.

Cancer cells are cells that divide relentlessly, forming solid tumors or flooding the blood with abnormal cells. Cell division is a normal process used by the body for growth and repair. A parent cell divides to form two daughter cells, and these daughter cells are used to build new tissue, or to replace cells that have died because of aging or damage. Healthy cells stop dividing when there is no longer a need for more daughter cells, but cancer cells continue to produce copies. They are also able to spread from one pail of the body to another in a process known as metastasis.

Carcinogen is any substance, radionuclide, or radiation that promotes carcinogenesis, the formation of cancer. This may be due to the ability to damage the genome or to the disruption of cellular metabolic processes. Several radioactive substances are considered carcinogens, but their carcinogenic activity is attributed to the radiation, for example gamma rays and alpha particles, which they emit. Common examples of non-radioactive carcinogens are inhaled asbestos, certain dioxins, and tobacco smoke. Although the public generally associates carcinogenicity with synthetic chemicals, it is equally likely to arise in both natural and synthetic substances. Carcinogens are not necessarily immediately toxic; thus, their effect can be insidious. Cancer is any disease in which normal cells are damaged and do not undergo programmed cell death as fast as they divide via mitosis. Carcinogens may increase the risk of cancer by altering cellular metabolism or damaging DNA directly in cells, which interferes with biological processes, and induces the uncontrolled, malignant division, ultimately leading to the formation of tumors. Usually, severe DNA damage leads to program cell death, but if the programmed cell death pathway is damaged, then the cell cannot prevent itself from becoming a cancer cell. There are many natural carcinogens. Aflatoxin B₁, which is produced by the fungus Aspergillus flavus growing on stored grains, nuts and peanut butter, is an example of a potent, naturally occurring microbial carcinogen. Certain viruses such as hepatitis B and human papilloma virus have been found to cause cancer in humans.

Cellular aging is generally defined as the progressive decline in the resistance to stress and other cellular damages, causing a gradual loss of cellular functions and resulting eventually in cell death.

Cellular agriculture is an interdisciplinary branch of science combining biology and engineering, focused on the production of agriculture products from cell cultures. Cellular agriculture combines disciplines such as biotechnology, tissue engineering, molecular biology, and synthetic biology to design new methods of producing proteins, fats, and tissues that would otherwise come from traditional agriculture. Cellular agriculture most often refers to the creation of animal products such as meat, milk, and eggs, produced in cell culture rather than raising and slaughtering farmed livestock. The most well known cellular agriculture concept is cultured meat.

Cell damage can occur as a result of an adverse stimulus, which disrupts the normal homeostasis of affected cells. Among other causes, this can be due to physical, chemical, an infectious, biological, nutritional or immunological factor. Cell damage can be reversible or irreversible. Depending on the extent of injury, the cellular response may be adaptive and where possible, homeostasis is restored. Cell death occurs when the severity of the injury exceeds the cell's ability to repair itself. Cell death is relative to both the length of exposure to a harmful stimulus and the severity of the damage caused. Cell death may occur by necrosis or apoptosis.

Cell function. The structural and functional characteristics of different types of cells are determined by the nature of the proteins present. The generalized cell functions include movement of substances across the cell membrane, cell division to make new cells, and protein synthesis.

Cell injury can occur when the limits of adaptive responses are exceeded cell injury occurs, initially reversible, then irreversible leading to cell death. Necrosis: severe cell swelling or cell rupture, denaturation and. coagulation of cytoplasmic proteins and breakdown of cell organelles.

Cell-cell interaction refers to the direct interactions between cell surfaces that play a crucial role in the development and function of multicellular organisms. These interactions allow cells to communicate with each other in response to changes in their microenvironment. This ability to send and receive signals is essential for the survival of the cell. Interactions between cells can be stable such as those made through cell junctions. These junctions are involved in the communication and organization of cells within a particular tissue. Others are transient or temporary such as those between cells of the immune system or the interactions involved in tissue inflammation. These types of intercellular interactions are distinguished from other types such as those between cells and the extracellular matrix. The loss of communication between cells can result in uncontrollable cell growth and cancer.

Crop is a plant or animal product that can be grown and harvested extensively for profit or subsistence. Crop may refer either to the harvested parts or to the harvest in a more refined state. Most crops are cultivated in agriculture or aquaculture. A crop is usually expanded to include macroscopic fungus (e.g. mushrooms), or alga (algaculture). Most crops are harvested as food for humans or fodder for livestock. Some crops are gathered from the wild (including intensive gathering, e.g. ginseng). Important non-food crops include horticulture, floriculture and industrial crops. Horticulture crops include plants used for other crops (e.g. fruit trees). Floriculture crops include bedding plants, houseplants, flowering garden and pot plants, cut cultivated greens, and cut flowers. Industrial crops are produced for clothing (fiber crops), biofuel (energy crops, algae fuel), or medicine (medicinal plants).

Cultured meat, also called clean meat, synthetic meat or in vitro meat, is meat grown from in vitro animals cell culture instead of from slaughtered animals It is a form of cellular agriculture. Cultured meat is produced using many of the same tissue engineering techniques traditionally used in regenerative medicine.

Disease is any condition, which results in the disorder of a structure or function in a living organism that is not due to any external injury. The study of disease is called pathology, which includes the study of cause. Disease is often construed as a medical condition associated with specific symptoms and signs. It may be caused by external factors such as pathogens or by internal dysfunctions, particularly of the immune system, such as an immunodeficiency, or by a hypersensitivity, including allergies and autoimmunity When caused by pathogens (e.g. malaria by Plasmodium ssp.), the term disease is often misleadingly used even in the scientific literature in place of its causal agent, the pathogen. This language habit can cause confusion in the communication of the cause-effect principle in epidemiology, and as such it should be strongly discouraged. In humans disease is often used more broadly to refer to any condition that causes pain, dysfunction, distress, social problems, or death to the person afflicted, or similar problems for those in contact with the person. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases can affect people not only physically, but also emotionally, as contracting and living with a disease can alter the affected person's perspective on life. Death due to disease is called death by natural causes. There are four main types of disease: infectious diseases, deficiency diseases, genetic diseases (both hereditary and non-hereditary), and physiological diseases. Diseases can also be classified as communicable and non-communicable. The deadliest diseases in humans are coronary artery disease (blood flow obstruction), followed by cerebrovascular disease and lower respiratory infections.

DNA damage is distinctly different from mutation, although both are types of error in DNA. DNA damage is an abnormal chemical structure in DNA, while a mutation is a change in the sequence of standard base pairs. DNA damages cause changes in the structure of the genetic material and prevents the replication mechanism from functioning and performing properly. DNA damage and mutation have different biological consequences. While most DNA damages can undergo DNA repair, such repair is not 100 percent efficient. Un-repaired DNA damages accumulate in non-replicating cells, such as cells in the brains or muscles of adult mammals and can cause aging. In replicating cells, such as cells lining the colon, errors occur upon replication past damages in the template strand of DNA or during repair of DNA damages.

These errors can give rise to mutations or epigenetic alterations. Both of these types of alteration can be replicated and passed on to subsequent cell generations. These alterations can change gene function or regulation of gene expression and possibly contribute to progression to cancer. Throughout the cell cycle there are various checkpoints to ensure the cell is in good condition to progress to mitosis. The three main checkpoints are at G1/s, G2/m, and at the spindle assembly checkpoint regulating progression through anaphase. G1 and G2 checkpoints involve scanning for damaged DNA. During S phase the cell is more vulnerable to DNA damage than any other part of the cell cycle. G2 checkpoint checks for damaged DNA and DNA replication completeness.

DNA photolyase is an evolutionary conserved protein domain. This domain binds a light harvesting cofactor. Deoxyribodipyrimidine photolyase (DNA photolyase) is a DNA repair enzyme. It binds to UV-damaged DNA containing pyrimidine dimers and, upon absorbing a near-UV photon (300 to 500 nm), breaks the cyclobutane ring joining the two pyrimidines of the dimer. DNA photolyase is an enzyme that requires two chromophore-cofactors for its activity: a reduced FADH2 and either 5,10-methenyltetrahydrofolate (5,10-MTFH) or an oxidized 8-hydroxy-5-deazaflavin (8-HDF) derivative (F420). The folate or deazaflavin chromophore appears to function as an antenna, while the FADH2 chromophore is thought to be responsible for electron transfer. On the basis of sequence similarities DNA photolyases can be grouped into two classes. The first class contains enzymes from Gram-negative and Gram-positive bacteria, the halophilic archaebacteria Halobacterium halobium, fungi and plants. Class 1 enzymes bind either 5,10-MTHF (E. coli, fungi, etc.) or 8-HDF (S. griseus, H. halobium). Proteins containing this domain also include Arabidopsis thaliana cryptochromes 1 (CRY1) and 2 (CRY2), which are blue light photoreceptors that mediate blue light-induced gene expression.

DNA repair is a collection of processes by which a cell identifies and corrects damage to the DNA molecules that encode its genome. In human cells, both normal metabolic activities and environmental factors such as radiation can cause DNA damage, resulting in as many as 1 million individual molecular lesions per cell per day. Many of these lesions cause structural damage to the DNA molecule and can alter or eliminate the cell's ability to transcribe the gene that the affected DNA encodes. Other lesions induce potentially harmful mutations in the cell's genome, which affect the survival of its daughter cells after it undergoes mitosis. As a consequence, the DNA repair process is constantly active as it responds to damage in the DNA structure, When normal repair processes fail, and when cellular apoptosis does not occur, irreparable DNA damage may occur, including double-strand breaks and DNA cross-linkages (interstrand crosslinks or ICLs). This can eventually lead to malignant tumors, or cancer as per the two hit hypothesis. The rate of DNA repair is dependent on many factors, including the cell type, the age of the cell, and the extracellular environment. A cell that has accumulated a large amount of DNA damage, or one that no longer effectively repairs damage incurred to its DNA, can enter one of three possible states: an irreversible state of dormancy, known as senescence; cell suicide, also known as apoptosis or programmed cell death; unregulated cell division, which can lead to the formation of a tumor that is cancerous. The DNA repair ability of a cell is vital to the integrity of its genome and thus to the normal functionality of that organism. Many genes that were initially shown to influence life span have turned out to be involved in DNA damage repair and protection.

DNA virus is a virus that has DNA as its genetic material and replicates using a DNA-dependent DNA polymerase. The nucleic acid is usually double-stranded DNA (dsDNA) but may also be single-stranded DNA (ssDNA), DNA viruses belong to either Group I or Group II of the Baltimore classification system for viruses. Single-stranded DNA is usually expanded to double-stranded in infected cells. Although Group VII viruses such as hepatitis B contain a DNA genome, they are not considered DNA viruses according to the Baltimore classification, but rather reverse transcribing viruses because they replicate through an RNA intermediate. Notable diseases like smallpox, herpes, and the chickenpox are caused by such DNA viruses.

Electromagnetic Spectrum consists of the full range of known electromagnetic waves, including gamma rays, X-rays, ultraviolet light, visible light, infrared light, microwaves, and radio waves. All of these are produced by various forms of oscillating charges or currents. Electromagnetic waves are transverse waves, which travel at the speed of tight and involve oscillating electric and magnetic fields at right angles to each other.

Gamma Rays are the most energetic known form of electromagnetic radiation, with frequencies of 3*10¹⁹ Hz (30 EHz) or higher and wavelengths of 10⁻¹¹ m (10 pm) or lower. They are produced from the decay of excited nuclei of radioactive elements. Gamma rays can penetrate through several centimeters of lead and large doses of them are harmful.

X-rays have frequencies ranging from 3*10¹⁶ to 3*10¹⁹ Hz (30 PHz to 30 EHz) and wavelengths from 10⁻¹¹ m to 10⁻⁸ m (10 pm to 10 nm). X-rays penetrate easily through some materials such as fat and muscle but not others such as bone, which makes them useful in medicine. Medical X-rays are produced by colliding high-energy electrons into a metal target, usually tungsten.

Ultraviolet Light (UV) light is light with higher frequency (lower wavelength) than visible light. UV light has frequencies ranging from 8*10¹⁴ to 3*10¹⁶ Hz (800 THz to 30 PHz) and wavelengths from 10⁻⁸ m to 3.8*10⁻⁷ m (10 nm to 380 nm). Prolonged exposure to UV light causes sunburn and can cause skin cancer. The earth's ozone layer absorbs most of the incoming UV light from the sun.

Visible Light is light we can see. Visible light has frequencies ranging from 4*10¹⁴ Hz to 8*10¹⁴ Hz (400 to 800 THz) and wavelengths from 3.8*10⁻⁷ m to 7.5*10⁻⁷ m (380 nm to 750 nm). Red light has the lowest frequency (longest wavelength) and violet light has the highest frequency (shortest wavelength) of visible light. White light is composed of the colors of the rainbow, each of which has a unique frequency or wavelength, as can be seen by diffracting white light through a prism.

Infrared Light (IR) light is light with lower frequency (higher wavelength) than visible light. IR light has frequencies ranging from 3*10¹¹ to 4*10¹⁴ Hz (300 GHz to 400 THz) and wavelengths from 7.5*10⁻⁷ m to 10⁻³ m (750 nm to 1 mm). Infrared light is emitted from all objects at or near room temperature in the form of blackbody radiation.

Microwaves have frequencies ranging from 3*10⁹ to 3*10¹¹ Hz (3 GHz to 300 GHz) and wavelengths from 10⁻³ m to 10⁻¹ in mm to 100 mm). Excitations from microwaves cause many molecules such as water to oscillate and heat up, which is how microwave ovens work. The primordial big bang radiation discovered by Penzias and Wilson in 1965 is in the microwave region.

Radio waves have frequencies ranging from 3*10¹ to 3*10⁹ Hz (30 Hz to 3 GHz) and wavelengths from 10⁻¹ m to 10⁷ m (100 mm to 10,000 km). Radio waves are produced from the alternating current of an LC circuit. Radio waves carry information through modulation. Radio, television, cell phones, and remote controls all use radio waves.

Electromagnetic wave, also known as electromagnetic radiation, is formed when an atomic particle moves, causing a combined electric field and magnetic field to oscillate, creating a combined wave pattern of the two forces. In the wave pattern, the magnetic and electric waves, although combined into one wave, oscillate at tight angles of each other. The electric wave oscillates vertically while the magnetic wave oscillates horizontally, but both waves move along a shared axis in the direction the combined wave is moving. Electromagnetic waves are usually measured in terms of frequency, wavelength, and energy, usually depending upon the size of the waves and the reason for the measurement. The longest waves, like radio and microwaves, are generally measured in frequency; the middle range of waves, like infrared, visible light and ultraviolet are measured by wavelength; and the shortest wavelengths, like x-rays arid gamma rays, are often measured by the amount of energy they produce. Using these different units to measure electromagnetic waves allow scientists to describe the waves without using numbers that are unwieldy or difficult to use in formulas. The frequency of an electromagnetic wave is measured by computing how many times the crest of a wave passes through a point in 1 second, the wavelength of an electromagnetic wave is the distance between two crests of the wave, and the energy of a wave is described by the amount of energy needed to move an electron.

Extracellular. In cell biology, molecular biology and related fields, the word extracellular (or sometimes extracellular space) means “outside the cell”. This space is usually taken to be outside the plasma membranes, and occupied by fluid. The term is used in contrast to intracellular (inside the cell). According to the gene ontology database the extracellular space is a cellular component defined as: “That part of a multicellular organism outside the cells proper, usually taken to be outside the plasma membranes, and occupied by fluid. For multicellular organisms, the extracellular space ⁻refers to everything outside a cell, but still within the organism (excluding the extracellular matrix). Gene products from a multi-cellular organism that are secreted from a cell into the interstitial fluid or blood can therefore be annotated to this term”. The composition of the extracellular space includes metabolites, ions, various proteins and non-protein substances (e.g. DNA, RNA, lipids, microbial products etc.) that might affect cellular function. For example, hormones, growth factors, cytokines and chemokines act by travelling the extracellular space towards biochemical receptors on cells. Other proteins that are active outside the cell are various enzymes, including digestive enzymes (Trypsin, Pepsin), extracellular proteinases (Matrix metalloproteinases, ADAMTSs, Cathepsins) and antioxidant enzymes (extracellular superoxide dismutase). Often, proteins present in the extracellular space are stored outside the cells by attaching to various extracellular matrix components (Collagens, Proteoglycans, etc.). In addition, extracellular matrix proteolytic products are also present in the extracellular space, especially in tissues undergoing remodeling.

Frequency is the number of occurrences of a repeating event per unit of time. It is also referred to as temporal frequency. which emphasizes the contrast to spatial frequency and angular frequency. The period is the duration of time of one cycle in a repeating event, so the period is the reciprocal of the frequency. For example, if a newborn baby's heart beats at a frequency of 120 times a minute, its period—the time interval between beats—is half a second (that is, 60 seconds divided by 120 beats). Frequency is an important parameter used in science and engineering to specify the rate of oscillatory and vibratory phenomena, such as mechanical vibrations, audio (sound) signals, radio waves, and light.

Frequencies of the present invention include two or more frequencies selected from 1, 2, 3, 4, 5, 6, 7, 8, 9, 10, 11, 12, 13, 14, or any range or value therein or between, comprising one or more of frequencies including two or more of 963, 852, 741, 639, 528, 417, 396, 288, 174, or harmonics, or subharmonics thereof, including pulses of EMFs, sound, and/or laser light including two or more of frequencies between 0.25 Hz and 11780000 Hz

Gene mutation. The DNA sequence of a gene can be altered in a number of ways. Gene mutations have varying effects on health, depending on where they occur and whether they alter the function of essential proteins.

Missense mutation, This type of mutation is a change in one DNA base pair that results in the substitution of one amino acid for another in the protein made by a gene.

Nonsense mutation. A nonsense mutation is also a change in one DNA base pair. Instead of substituting one amino acid for another, however, the altered DNA sequence prematurely signals the cell to stop building a protein. This type of mutation results in a shortened protein that may function improperly or not at all.

Insertion. An insertion changes the number of DNA bases in a gene by adding a piece of DNA. As a result, the protein made by the gene may not function properly.

Deletion. A deletion changes the number of DNA bases by removing a piece of DNA. Small deletions may remove one or a few base pairs within a gene, while larger deletions can remove an entire gene or several neighboring genes. The deleted DNA may alter the function of the resulting protein(s).

Duplication. A duplication consists of a piece of DNA that is abnormally copied one or more times. This type of mutation may alter the function of the resulting protein.

Frameshift mutation. This type of mutation occurs when the addition or loss of DNA bases changes a gene's reading frame. A reading frame consists of groups of 3 bases that each code for one amino acid. A frameshift mutation shifts the grouping of these bases and changes the code for amino acids. The resulting protein is usually nonfunctional. insertions, deletions, and duplications can all be frameshift mutations.

Repeat expansion. Nucleotide repeats are short DNA sequences that are repeated a number of times in a row. For example, a trinucleotide repeat is made up of 3-base-pair sequences, and a tetranucleotide repeat is made up of 4-base-pair sequences. A repeat expansion is a mutation that increases the number of times that the short DNA sequence is repeated. This type of mutation can cause the resulting protein to function improperly.

Glycocalyx, also known as the pericellular matrix, is a glycoprotein and glycolipid covering that surrounds the cell membranes of some bacteria, epithelia and other cells. Most animal epithelial cells have a fuzz-like coat on the external surface of their plasma membranes. This coating consists of several carbohydrate moieties of membrane glycolipids and glycoproteins, which serve as backbone molecules for support. Generally, the carbohydrate portion of the glycolipids found on the surface of plasma membranes helps these molecules contribute to cell-cell recognition, communication, and intercellular adhesion. The glycocalyx is a type of identifier that the body uses to distinguish between its own healthy cells and transplanted tissues, diseased cells, or invading organisms. Included in the glycocalyx are cell-adhesion molecules that enable cells to adhere to each other and guide the movement of cells during embryonic development. The glycocalyx plays a major role in regulation of endothelial vascular tissue, including the modulation of red blood cell volume in capillaries. The slime on the outside of a fish is an example of glycocalyx.

Hematologic conditions. There are many conditions of or affecting the human hematologic system—the biological system that includes plasma, platelets, leukocytes, and erythrocytes, the major components of blood and the bone marrow. An anemia is a decrease in number of red blood cells (RBCs) or less than the normal quantity of hemoglobin in the blood. However, it can include decreased oxygen-binding ability of each hemoglobin molecule due to deformity or lack in numerical development as in some other types of hemoglobin deficiency. Anemia is the most common disorder of the blood. There are several kinds of anemia, produced by a variety of underlying causes. Anemia can he classified in a variety of ways, based on the morphology of RBCs, underlying etiologic mechanisms, and discernible clinical spectra, to mention a few. The three main classes of anemia include excessive blood loss (acutely such as a hemorrhage or chronically through low-volume loss), excessive blood cell destruction (hemolysis) or deficient red blood cell production (ineffective hematopoiesis).

Hertz (symbol: Hz) is the derived unit of frequency in the International System of Units (SI) and is defined as one cycle per second. It is named for Heinrich Rudolf Hertz, the first person to provide conclusive proof of the existence of electromagnetic waves. Hertz are commonly expressed in multiples: kilohertz (10³ Hz, kHz), megahertz (10⁶ Hz, MHz), gigahertz (10⁹ Hz, GHz), and terahertz (10¹² Hz, THz). Some of the unit's most common uses are in the description of sine waves and musical tones, particularly those used in radio- and audio-related applications. It is also used to describe the speeds at which computers and other electronics are driven. One hertz simply means “one cycle per second” (typically that which is being counted is a complete cycle); 100 Hz means “one hundred cycles per second,” and so on. The unit may be applied to any periodic event—for example, a clock might be said to tick at 1 Hz, or a human heart might be said to beat at 1.2 Hz.

Homeostasis can be defined as the stable condition of an organism and of its internal environment; or as the maintenance or regulation of the stable condition, or its equilibrium; or simply as the balance of bodily functions. The stable condition is the condition of optimal functioning for the organism, and is dependent on many variables, such as body temperature and fluid balance, being kept within certain pre-set limits. Other variables include the pH of extracellular the concentrations of sodium, potassium and calcium ions, as well as that of the blood sugar level, and these need to be regulated despite changes in the environment, diet, or level of activity. Each of these variables is controlled by one or more regulators or homeostatic mechanisms, which together maintain life. Homeostasis is brought about by a natural resistance to change in the optimal conditions, and equilibrium is maintained by many regulatory mechanisms. All homeostatic control mechanisms have at least three interdependent components for the variable being regulated: a receptor, a control center, and an effector. The receptor is the sensing component that monitors and responds to changes in the environment, either external or internal. Receptors include thereto receptors, and mechanoreceptors. Control centers include the respiratory center, and the renin-angiotensin system. An effector is the target acted on, to bring about the change back to the normal state. At the cellular level, receptors include nuclear receptors that bring about changes in gene expression through up-regulation or down-regulation, and act in negative feedback mechanisms. An example of this is in the control of bile acids in the liver.

Host-pathogen interaction is defined as how microbes or viruses sustain themselves within host organisms on a molecular, cellular, organismal or population level. This term is most commonly used to refer to disease-causing microorganisms although they may not cause illness in all hosts. Because of this, the definition has been expanded to how known pathogens survive within their host, whether they cause disease or not. On the molecular and cellular level, microbes can infect the host and divide rapidly, causing disease by being there and causing a homeostatic imbalance in the body, or by secreting toxins, which cause symptoms to appear. Viruses can also infect the host with virulent DNA, which can affect normal cell processes (transcription, translation, etc.), protein folding, or evading the immune response.

Human electrical energy is generated by chemical processes in nerve cells. Billions of nerve impulses travel throughout the human brain and nervous system. A nerve impulse is a wave of electrical activity that passes from one end of nerve cell to another. Each impulse is the same size that carries information about the intensity of the nerve signal. Neurons, basic unit of nervous system, are responsible for sending, receiving, and interpreting information from all parts of the body. The human body is a complex and carefully-balanced superhighway of cells, tissues and fluids that direct an incomprehensible array of electrical impulses almost every second. This is only possible due to a homeostatic environment where electricity is well conducted to carry the signals to their intended destinations. They key to maintaining this conductive superhighway lies within the electrolytes. Electrolytes regulate our nerve and muscle function, the human body's hydration, blood pH, blood pressure and the rebuilding of damaged tissues. Various mechanisms exist in the human body that keeps the concentrations of different electrolytes under strict control.

Electrical Activity of Cells. One of the simplest physiological units is the cell. It has the power of maintaining itself alive given suitable surroundings. The endocrine pancreas consists of clusters of cells called islets of Langerhans. Most of the cells in an islet are insulin-secreting β-cells. Electrical impulses or action potentials are generated in bursts when the bath or blood glucose level is in the stimulatory range. Insulin insufficiency is generally a result of β-cell damage by autoimmunity. Closing adenosine triphosphate (ATP)-sensitive K+ channels (KATP) in response to glucose increase is generally considered the initial event that depolarizes the β-cell membrane and activates the voltage-dependent Ca2+ channels, which triggers the release of insulin. However, glucose is unable to extract insulin release if electrical activity and the accompanying Ca2+ influx are prevented. Thus beta-cell electrical activity is a key element in the series of steps culminating in glucose-induced insulin secretion.

Electrical Activity of Organs in vitro, in vivo, or in situ. Brain—an organ of soft nervous tissue contained in the skull of vertebrates, functioning as the coordinating center of sensation, intellectual and nervous activity. Human brain cells fire electrical impulses to communicate with one another. Ion channels located in the membranes, some with calcium and some with potassium; work in sync to deliver messages throughout the brain. Within a single neuron, information is conducted through electrical signaling. When a neuron is stimulated, an electrical impulse, called an action potential, moves along the neuron axon. Action potential then enables signals to travel very rapidly along the neuron fiber. When large numbers of neurons show synchronized activity, the electric fields generated can be large enough to detect outside the skull, using Electroencephalography (EEG) or Magnetoencephalography (MEG).

Human parasites are divided into endoparasites, which cause infection inside the body, and ectoparasites, which cause infection superficially within the skin. The cysts and eggs of endoparasites may be found in feces, which aids in the detection of the parasite in the human host while also providing the means for the parasitic species to exit the current host and enter other hosts.—Although there are a number of ways in which humans can contract parasitic infections, observing basic hygiene and cleanliness tips can reduce its probability. The most accurate diagnosis is by qPer DNA antigen assay, not generally available by primary care physicians in the USA: most labs offer research only service.

Human pathogen is a pathogen (microbe or microorganism such as a virus, bacterium, prion, or fungus) that causes disease in humans. The human physiological defense against common pathogens (such as Pneumocystis) is mainly the responsibility of the system with help by some of the body's normal flora and fauna. However, if the immune system or “good” microbiota are damaged. in any way (such as by chemotherapy, human immunodeficiency virus (HIV), or antibiotics being taken to kill other pathogens), pathogenic bacteria that were being held at bay can proliferate and cause harm to the host. Such cases are called opportunistic infections. Some pathogens (such as the bacterium Yersinia pestis, which may have caused the Black Plague, the Variola virus, and the malariaprotozoa) have been responsible for massive numbers of casualties and have had numerous effects on afflicted groups.

Hyperplasia or hypergenesis, is an increase in the amount of organic tissue that results from cell proliferation. It may lead to the gross enlargement of an organ and the term is sometimes confused with benign neoplasia or benign tumor. Hyperplasia. is a common preneoplastic response to stimulus. Microscopically, cells resemble normal cells but are increased in numbers, Sometimes cells may also be increased in size (hypertrophy). Hyperplasia is different from hypertrophy in that the adaptive cell change in hypertrophy is an increase in the size of cells, whereas hyperplasia. involves an increase in the number of cells.

Infrared radiation (IR) is electromagnetic radiation (EMR) with longer wavelengths than those of visible light. and is therefore generally invisible to the human eye (although IR at wavelengths up to 1050 nm from specially pulsed lasers can be seen by humans under certain conditions). It is sometimes called infrared light. IR wavelengths extend from the nominal red edge of the visible spectrum at 700 nanometers (frequency 430 THz), to 1 millimeter (300 GHz) Most of the thermal radiation emitted by objects near room temperature is infrared. Like all EMR, IR carries radiant energy, and behaves both like a wave and like its quantum particle, the photon.

Intense pulsed light (IPL) is a technology used by cosmetic and medical practitioners to perform various skin treatments for aesthetic and therapeutic purposes, including hair removal, photo rejuvenation (e.g. the treatment of skin pigmentation, sun damage, and thread veins) as well as to alleviate dermatologic diseases such as acne. IPL is increasingly used in optometry and ophthalmology as well, to treat evaporative dry eye disease due to meibomian gland dysfunction. The technology uses a light-powered, hand-held, computer-controlled flashgun to deliver an intense, visible, broad-spectrum pulse of light, generally in the visible spectral range of 400 to 1200 nm. Various cutoff filters are commonly used to selectively filter out lower wavelengths, especially potentially damaging ultra violet light. The resulting light has a spectral range that targets specific structures and chromophores (e.g. melanin in hair, or oxyhemoglobin in blood vessels) that are heated to destruction and reabsorbed by the body. IPL shares some similarities with laser treatments, in that they both use light to heat and destroy their targets. But unlike lasers that use a single wavelength (color) of light which typically matches only one chromophore, and hence only one condition, IPL uses a broad spectrum that when used with filters, allows it to be used against several conditions. This can be achieved when the IPL technician selects the appropriate filter that matches a specific chromophore.

Internal organs in the human body include: heart, lungs, stomach, kidney, diaphragm, spleen, liver, pancreas, large and small intestine, gall bladder, bladder and brain.

Intracellular. In cell biology, molecular biology and related fields, the word intracellular means “inside the cell”. It is used in contrast to extracellular (outside the cell). The cell membrane (and, in many organisms, the cell wall) is the barrier between the two, and chemical composition of intra- and extracellular milieu (Milieu Intérieur) can be radically different.

Macrophages are a type of white blood cell that engulfs and digests cellular debris, foreign substances, microbes, cancer cells, and anything else that does not have the types of proteins specified to healthy body cells on its surface in a process called phagocytosis. These large phagocytes are found in essentially all tissues, where they patrol for potential pathogens; and cellular markers of aging by amoeboid movement. They take various forms (with various names) throughout the body (e.g., histiocytes, Kupffer cells, alveolar macrophages, microglia, and others), but all are part of the mononuclear phagocyte system. Besides phagocytosis, they play a critical role in non-specified. defense (innate immunity) and also help initiate specified defense mechanisms (adaptive immunity) by recruiting other immune cells such as lymphocytes, For example, they are important as antigen presenters to T cells, In humans, dysfunctional macrophages cause severe diseases such as chronic granulomatous disease that result in frequent infections. Beyond increasing inflammation and stimulating the immune system, macrophages also play an important anti-inflammatory role and can decrease immune reactions through the release of cytokines. Macrophages that encourage inflammation are called M1 macrophages, whereas those that decrease inflammation and encourage tissue repair are called M2 macrophages, This difference is reflected in their metabolism; M1 macrophages have the unique ability to metabolize arginine to the “killer” molecule nitric oxide, whereas rodent M2 macrophages have the unique ability to metabolize arginine to the “repair” molecule ornithine. However, this dichotomy has been recently questioned as further complexity has been discovered. Human macrophages are about 21 micrometers (0.00083 in) in diameter and produced by the differentiation of monocytes in tissues. They can be identified using flow cytometry or immunohistochemical staining by their specific expression of proteins such as CD14, CD40, CD 11b, CD64, F4/80 (mice)/EMR1 (human), lysozyme M, MAC-1/MAC-3 and CD68.

Malignancy (from Latin male, meaning ‘badly’, and -gnus, meaning ‘born’) is the tendency of a medical condition to become progressively worse. Malignancy is most familiar as a characterization of cancer. A malignant tumor contrasts with a non-cancerous benign tumor in that a malignancy is not self-limited in its growth, is capable of invading into adjacent tissues, and may be capable of spreading to distant tissues, A benign tumor has none of those properties. Malignancy in cancers is characterized by anaplasia, invasiveness, and metastasis. Malignant tumors are also characterized by genome instability, so that cancers, as assessed by whole genome sequencing, frequently have between 10,000 and 100,000 mutations in their entire genomes. Cancers usually show tumor heterogeneity, containing multiple sub-clones. They also frequently have reduced expression of DNA repair enzymes due to epigenetic methylation of DNA repair genes or altered microRNAs that control DNA repair gene expression.

Metastasis is a pathogenic agent's spread from an initial or primary site to a different or secondary site within the host's body; it is typically spoken of as such spread by a cancerous tumor. The newly pathological sites. then, are metastases (mets). Cancer occurs after cells are genetically altered to proliferate rapidly and indefinitely. This uncontrolled proliferation by mitosis produces a primary heterogeneic tumor. The cells which constitute the tumor eventually undergo metaplasia, followed by anaplasia then dysplasia, resulting in a malignant phenotype. This malignancy allows for invasion into the circulation, followed by invasion to a second site for tumor genesis. Some cancer cells known as circulating tumor cells acquire the ability to penetrate the walls of lymphatic or blood vessels, after which they are able to circulate through the bloodstream to other sites and tissues in the body. This process is known (respectively) as lymphatic or hematogenous spread. After the tumor cells come to rest at another site, they re-penetrate the vessel or walls and continue to multiply, eventually forming another clinically detectable tumor. This new tumor is known as a metastatic (or secondary) tumor. Metastasis is one of the hallmarks of cancer, distinguishing it from benign tumors. Most cancers can metastasize, although in varying degrees. Basal cell carcinoma for example rarely metastasizes. When tumor cells metastasize, the new tumor is called a secondary or metastatic tumor, and its cells are similar to those in the original or primary tumor. This means that if breast cancer metastasizes to the lungs, the secondary tumor is made up of abnormal breast cells, not of abnormal lung cells. The tumor in the lung is then called metastatic breast cancer, not lung cancer. Metastasis is a key element in cancer staging systems such as the TNM staging system, where it represents the “M”. In overall stage grouping, metastasis places a cancer in Stage IV.

Molecular vibration occurs when atoms in a molecule are in periodic motion while the molecule as a whole has constant translational and rotational motion. The frequency of the periodic motion is known as a vibration frequency, and the typical frequencies of molecular vibrations range from less than 10¹³ to approximately 10¹⁴ Hz, corresponding to wavenumbers of approximately 300 to 3000 cm⁻¹. In general, a molecule with N atoms has 3N-6 normal modes of vibration, but a linear molecule has 3N-5 such modes, because rotation about its molecular axis cannot be observed. A diatomic molecule has one normal mode of vibration. The normal modes of vibration of polyatomic molecules are independent of each other but each normal mode will involve simultaneous vibrations of different parts of the molecule such as different chemical bonds. A molecular vibration is excited when the molecule absorbs a quantum of energy, E, corresponding to the vibration's frequency, v, according to the relation E=h v (where h is Planck's constant). A fundamental vibration is excited when one such quantum of energy is absorbed by the molecule in its ground state.

Mutation is the permanent alteration of the nucleotide sequence of the genome of an organism, virus, or extrachromosomal DNA or other genetic elements. Mutations result from errors during DNA replication (especially during Meiosis) or other types of damage to DNA (such as may be caused by exposure to radiation or carcinogens), which then may undergo error-prone repair (especially microhomology-mediated end joining), or cause an error during other forms of repair, or else may cause an error during replication (translesion synthesis). Mutations may also result from insertion or deletion of segments of DNA due to mobile genetic elements. Mutations may or may not produce discernible changes in the observable characteristics (phenotype) of an organism. Mutations play a part in both normal and abnormal biological processes including: evolution, cancer, and the development of the immune system, including junctional diversity. The genomes of RNA viruses are based on RNA rather than DNA. The RNA viral genome can be double stranded (as in DNA) or single stranded. In some of these viruses (such as the single stranded human immunodeficiency virus) replication occurs quickly and there are no mechanisms to check the genome for accuracy. This error-prone process often results in mutations. Mutation can result in many different types of change in sequences. Mutations in genes can either have no effect, alter the product of a gene, or prevent the gene from functioning properly or completely. Mutations can also occur in nongenic regions.

Necrosis is a form of cell injury which results in the premature death of cells c by autolysis. Necrosis is caused by factors external to the cell or tissue, such as infection, toxins, or trauma, which result in the unregulated digestion of cell components. In contrast, apoptosis is a naturally occurring programmed and targeted cause of cellular death. While apoptosis often provides beneficial effects to the organism, necrosis is almost always detrimental and can be fatal. Cellular death due to necrosis does not follow the apoptotic signal transduction pathway, but rather various receptors are activated, and result in the loss of cell membrane integrity and an uncontrolled release of products of cell death into the extracellular space. This initiates in the surrounding tissue an inflammatory response, which attracts leukocytes and nearby phagocytes, which eliminate the dead cells by phagocytosis. However, microbial damaging substances released by leukocytes would create collateral damage to surrounding tissues. This excess collateral damage inhibits the healing process. Thus, untreated necrosis results in a build-up of decomposing dead tissue and cell debris at or near the site of the cell death.

Pathogen is an infectious thing, such as a vines, bacteria, fungi or parasite, winch causes a disease. This ability is called pathogenicity.

Pathogenic bacteria are bacteria that can cause infection. This article deals with human pathogenic bacteria. Although most bacteria are harmless or often beneficial, some are pathogenic, with the number of species estimated as fewer than 100 that are seen to cause infectious diseases in humans. By contrast, several thousand species exist in the human digestive system.

Pathogenic fungi are fungi that cause disease in humans or other organisms. Approximately :300 fungi are known to be pathogenic to humans. The study of fungi pathogenic to humans is called “medical mycology”. Although fungi are eukaryotic, many pathogenic fungi are microorganisms. The study of fungi and other organisms pathogenic to plants is called plant pathology.

Photolyases (EC 4.1.99.3) are DNA repair enzymes that repair damage caused by exposure to ultraviolet light. These enzymes require visible light (from the violet/blue end of the spectrum) both for their own activation and for the actual DNA repair. The DNA repair mechanism involving photolyases is called photo reactivation. Photolyase is a phylogenetically old. enzyme, which is present and functional in many species, from the bacteria to the fungito plants and

to the animals. Photolyase is particularly important in repairing UV induced damage in plants. The photolyase mechanism is no longer working in humans and other placental mammals that instead rely on the less efficient nucleotide excision repair mechanism. Photolyases bind complementary DNA strands and break certain types of pyrimidine dimers that arise when a pair of thymine or cytosine bases on the same strand of DNA become covalently linked. The bond length of this dimerization is shorter than the bond length of normal B-DNA structure, which produces an incorrect template for replication and transcription. The more common covalent linkage involves the formation of a cyclobutane bridge. Photolyases have a high affinity for these lesions and reversibly bind and convert them back to the original bases.

Pulse-amplitude modulation (PAM), is a form of signal modulation where the message information is encoded in the amplitude of a series of signal pulse. It is an analog pulse modulation scheme in which the amplitudes of a train of carrier pulses are varied according to the sample value of the message signal. Demodulation is performed by detecting the amplitude level of the carrier at every single period.

Pulse-density modulation, or PDM, is a form of modulation used to represent an analog signal with a binary signal. In a PDM signal, specific amplitude values are not encoded into code words of pulses of different weight as they would be in pulse-code modulation (PCM); rather, the relative density of the pulses corresponds to the analog signal's amplitude. The output of a 1-bit DAC is the same as the PDM encoding of the signal. Pulse-width modulation (PWM) is a special case of PDM where the switching frequency is fixed and all the pulses corresponding to one sample are contiguous in the digital signal. For a 50 percent voltage with a resolution of 8-bits, a PWM waveform will turn on for 128 clock cycles and then off for the remaining 128 cycles. With PDM and the same clock rate the signal would alternate between on and off every other cycle. The average is 50 percent for both waveforms, but the PDM signal switches more often. For 100 percent or 0 percent level, they are the same.

Pulse duration. In signal processing and telecommunication, pulse duration is the interval between the time, during the first transition, that the amplitude of the pulse reaches a specified fraction (level) of its final amplitude, and the time the pulse amplitude drops, on the last transition, to the same level. The interval between the 50 percent points of the final amplitude is usually used to determine or define pulse duration, and this is understood to be the case unless otherwise specified. Other fractions of the final amplitude, e.g., 90 percent or 1/e, may also he used, as may the root mean square (rms) value of the pulse amplitude. The duration of optical pulses (also called pulse width or pulse length) can vary in a huge range: By modulating a continuous-wave light source, e.g., with an electro-optic modulator, pulses with durations from some tens of picoseconds to arbitrarily high values can be generated. Gain switching e.g. of laser diodes leads to pulses with durations down to a few nanoseconds or even to some hundred picoseconds. Pulse durations from Q-switched lasers typically vary between 100 ps and hundreds of nanoseconds. Mode-locked lasers can generate pulses with durations between ≈5 fs and hundreds of picoseconds. high harmonic generation allows the formation of single attosecond pulses or attosecond pulse trains, with pulse durations of a few hundred attoseconds or even below 100 as.

Pulsed operation of lasers refers to any laser not classified as continuous wave, so that the optical power appears in pulses of some duration at some repetition rate. This encompasses a wide range of technologies addressing a number of different motivations. Some lasers are pulsed simply because they cannot be run in continuous mode. in other cases the application requires the production of pulses having as large energy as possible. Since the pulse energy is equal to the average power divided by the repetition rate, this goal can sometimes be satisfied by lowering the rate of pulses so that more energy can be built up in between pulses. In laser ablation for example, a small volume of material at the surface of a work piece can be evaporated if it is heated in a very short time, whereas supplying the energy gradually would allow for the heat to be absorbed into the bulk of the piece, never attaining a sufficiently high temperature at a particular point. Other applications rely on the peak pulse power (rather than the energy in the pulse), especially in order to obtain nonlinear optical effects. For a given pulse energy, this requires creating pulses of the shortest possible duration utilizing techniques such as Q-switching. The optical bandwidth of a pulse cannot be narrower than the reciprocal of the pulse width. In the case of extremely short pulses, that implies lasing over a considerable bandwidth, quite contrary to the very narrow bandwidths typical of CW lasers. The lasing medium in some dye lasers and vibronic solid-state lasers produces optical gain over a wide bandwidth, making a laser possible which can thus generate pulses of light as short as a few femtoseconds.

Q-switching. In a Q-switched laser, the population inversion is allowed to build up by introducing loss inside the resonator, which exceeds the gain of the medium; this can also be described as a reduction of the quality factor or ‘Q’ of the cavity. Then, after the pump energy stored in the laser medium has approached the maximum possible level, the introduced loss mechanism (often an electro- or acousto-optical element) is rapidly removed (or that occurs by itself in a passive device) allowing lasing to begin which rapidly obtains the stored energy in the gain medium. This results in a short pulse incorporating that energy, and thus a high peak power, Mode-locking. A mode-locked laser is capable of emitting extremely short pulses on the order of tens of picoseconds down to less than 10 femtoseconds. These pulses will repeat at the round trip time, that is, the time that it takes light to complete one round trip between the mirrors comprising the resonator. Due to the Fourier limit (also known as energy-time uncertainty), a pulse of such short temporal length has a spectrum spread over a considerable bandwidth. Thus such a gain medium must have a gain bandwidth sufficiently broad to amplify those frequencies. An example of a suitable material is titanium-doped, artificially grown sapphire (Ti:sapphire), which has a very wide gain bandwidth and can thus produce pulses of only a few femtoseconds duration. Such mode-locked lasers are a most versatile tool for researching processes occurring on extremely short time scales (known as femtosecond physics, femtosecond chemistry and ultrafast science), for maximizing the effect of nonlinearity in optical materials (e.g. in second-harmonic generation, parametric down-conversion, optical parametric oscillators and the like) due to the large peak power, and in ablation applications. Again, because of the extremely short pulse duration, such a laser will produce pulses, which achieve an extremely high peak power. Pulsed pumping. Another method of achieving pulsed laser operation is to pump the laser material with a source that is itself pulsed, either through electronic charging in the case of flash lamps, or another laser, which is already pulsed. Pulsed pumping was historically used with dye lasers where the inverted population lifetime of a dye molecule was so short that a high energy, fast pump was needed. The way to overcome this problem was to charge up large capacitors, which are then switched to discharge through flash lamps, producing an intense flash. Pulsed pumping is also required for three-level lasers in which the lower energy level rapidly becomes highly populated preventing further lasing until those atoms relax to the ground state. These lasers, such as the excimer laser and the copper vapor laser, can never be operated in CW mode. Pulsed lasers in soft-tissue surgery. When a laser beam comes into contact with soft-tissue, one important factor is to not overheat surrounding tissue, so necrosis can be prevented. Laser pulses must be spaced out to allow for efficient tissue cooling (thermal relaxation time) between pulses.

Pulse Repetition rate (PRR). When the laser is running in pulsed anode the number of pulses per second or frequency of pulses is known as the Pulse Repetition rate (PRR). The tem PRR and pulse frequency are commonly used. In welding this tends to be in the 10's of Hz, in fine cutting in the 100-1000's of Hz, and in marking or micro machining in the 10000's of Hz.

Pulse Repetition Frequency. The typical values of PRP in clinical echo are form 100 microseconds to 1 millisecond. A related parameter to PRP is the Pulse Repetition Frequency or PRF. PRP and PRF are reciprocal to each other. PRF is the number of pulses that occur in 1 second. When the laser is running in pulsed mode the number of pulses per second or frequency of pulses is known as the Pulse Repetition rate (PRR). The term PRR and pulse frequency are commonly used.

Pulse-width modulation (PWM), or pulse-duration modulation (PDM), is a modulation technique used to encode a message into a pulsing signal. Although this modulation technique can be used to encode information for transmission, its main use is to allow the control of the power supplied to electrical devices, especially to inertial loads such as motors. In addition, PWM is one of the two principal algorithms used in photovoltaic solar battery chargers, the other being maximum power point tracking. The average value of voltage (and current) fed to the load is controlled by turning the switch between supply and load on and off at a fast rate. The longer the switch is on compared to the off periods, the higher the total power supplied to the load. The PWM switching frequency has to be much higher than what would affect the load (the device that uses the power), which is to say that the resultant waveform perceived by the load must be as smooth as possible. The rate (or frequency) at which the power supply must switch can vary greatly depending on load and application, for example, Switching has to be done several times a minute in an electric stove; 120 Hz in a lamp dimmer;

between a few kilohertz (kHz) and tens of kHz for a motor drive; and well into the tens or hundreds of kHz in audio amplifiers and computer power supplies. The term duty cycle describes the proportion of ‘on’ time to the regular interval or ‘period’ of time; a low duty cycle corresponds to low power, because the power is off for most of the time. Duty cycle is expressed in percent, 100 percent being fully on. The main advantage of PWM is that power loss in the switching devices is very low. When a switch is off there is practically no current, and when it is on and power is being transferred to the load, there is almost no voltage drop across the switch. Power loss, being the product of voltage and current, is thus in both cases close to zero. PWM also works well with digital controls, which, because of their on/off nature, can easily set the needed duty cycle. PWM has also been used in certain communication systems where its duty cycle has been used to convey information over a communications channel.

Resonance is a phenomenon in which a vibrating system or external force drives another system to oscillate with greater amplitude at pulsed resonant frequencies. Frequencies at which the response amplitude is a relative maximum are known as the system's resonant frequencies or sound frequencies. At resonant frequencies in Hz, small periodic driving forces have the ability to produce large amplitude oscillations, due to the storage of vibrational energy. Resonance occurs when a system is able to store and easily transfer energy between one or more different storage modes (such as kinetic energy and potential energy in the case of a simple pendulum).

Sound energy is a form of energy associated with the vibration of matter. The SI unit of sound energy is the joule (J). Sound is a mechanical wave and as such consists physically in oscillatory elastic compression and in oscillatory displacement of a fluid.

Sound frequencies are often simplified to a description in terms of sinusoidal plane waves, which are characterized by these generic properties: Frequency, or its inverse, wavelength. Amplitude, sound pressure or Intensity. Speed of sound.

Specified absorption rate (SAR) is a measure of the rate at which energy is absorbed by the human body when exposed to a radio frequency (RF) electromagnetic field. It can also refer to absorption of other forms of energy by tissue, including ultrasound. It is defined as the power absorbed per mass of tissue and has units of watts per kilogram (W/kg). SAR is usually averaged either over the whole body, or over a small sample volume (typically 1 g or 10 g of tissue). The value cited is then the maximum level measured in the body part studied over the stated volume or mass.

Tumor cells are an abnormal growth of tissue resulting uncontrolled growth of cells and serving no function within the body; a cancerous growth. Tumors can he benign (unlikely to spread to other body parts) or malignant (likely to spread).

Ultrasound is sound frequencies with frequencies higher than the upper audible limit of human hearing. Ultrasound is no different from ‘normal’ (audible) sound in its physical properties, except in that humans cannot hear This limit varies from person to person and is approximately 20 kilohertz (20,000 hertz) in healthy, young adults. Ultrasound devices operate with frequencies from 20 kHz up to several gigahertz. Ultrasound is used in many different fields. Ultrasonic devices are used to detect objects and measure distances. Ultrasound imaging or sonography is often used in medicine. In the nondestructive testing of products and structures, ultrasound is used to detect invisible flaws. Industrially, ultrasound is used for cleaning, mixing, and to accelerate chemical processes.

Vibration or oscillation means quickly moving back and forth (or up and down) about a point of equilibrium. The vibration may be periodic (having a pattern) or random. Something that is vibrating may shake at the same time. If it vibrates in a regular way, it may produce a musical note because it can make the air vibrate. This vibration will send sound. waves to the ear and to the brain. In structural engineering including earthquake engineering, vibrations may be bad. They may cause the structure to fail. The time it takes a vibrating object to go back and forth completely is the period. The number of back-and-forth movements in one second is its frequency, measured in Hertz (Hz). The farthest possible distance from the equilibrium point is the amplitude.

Waves are described and measured by five wave parameters: the period, the frequency, the amplitude, the wavelength, and the speed. The period of a wave is the time it takes to complete one cycle. The frequency is just the opposite; its the number of wave cycles that arc completed in one second.

Wavelength of a sinusoidal wave is the spatial period of the wave—the distance over which the wave's shape repeats, and thus the inverse of the spatial frequency. It is usually determined by considering the distance between consecutive corresponding points of the same phase, such as crests, troughs, or zero crossings and is a characteristic of both traveling waves and standing waves, as well as other spatial wave patterns. Wavelength is commonly designated by the Greek letter lambda (λ). The concept can also be applied to periodic waves of non-sinusoidal shape. The term wavelength is also sometimes applied, to modulated waves, and to the sinusoidal envelopes of modulated waves or waves formed by interference of several sinusoids. Assuming a sinusoidal wave moving at a fixed wave speed, wavelength is inversely proportional to frequency of the wave: waves with higher frequencies have shorter wavelengths, and lower frequencies have longer wavelengths. Wavelength depends on the medium (for example, vacuum, air, or water) that a wave travels through, Examples of wave-like phenomena are frequencies in Hz, light, water waves and periodic electrical signals in a conductor. A sound wave is a variation in air pressure, while in light and other electromagnetic radiation the strength of the electric and the magnetic field vary. Water waves are variations in the height of a body of water. In a crystal lattice vibration, atomic positions vary. Wavelength is a measure of the distance between repetitions of a shape feature such as peaks, valleys, or zero-crossings, not a measure of how far any given particle moves.

### LIST OF DISEASES, ABNORMAL CONDITIONS, DISORDERS OR OTHER MEDICAL CONDITIONS AND CORRESPONDING FREQUENCIES IN HERTZ (HZ) THAT IMPROVE ONE OR MORE PROPERTIES OR FUNCTIONS OF CELLS, TISSUES, PROTEINS, FATS, OR ORGANS IN VITRO, IN VIVO, OR IN SITU

Abdominal_inflammation—2720, 2489, 2170, 2000, 1865, 1800, 1600, 1550, 880, 832, 802, 787, 776, 727, 660, 465, 450, 444, 440, 428, 380, 250, 146, 125, 95, 72, 20, 1.2; Abdominal_pain—10000, 3000, 95, 3; Abscesses (also use Staphylococcus aureus and see Listeriose)—2720, 2170, 1865, 1550, 880, 802, 787, 727, 500, 444, 190; Abscesses_1—2720, 2170, 1865, 1550, 880, 802, 787, 760, 727, 690, 660, 500, 465, 450, 444, 428, 190; Abscesses_secondary—1.550, 802, 760, 660, 465, 450, 444, 428; Acidosis (hyperacidity. Also use a calcium magnesium supplement long term)—10000, 880, 802, 787, 776, 727, 146, 20; Acne (run 564 for 6 min)—2720, 2170, 1800, 1600, 1550, 1552, 1500, 802, 880, 778, 787, 760, 741, 727, 660, 564, 465, 450, 444, 428; Aconite (used to stimulate lymphocyte production)—3347, 5611, 2791; Actinobacillus (a potentially pathogenic bacteria found in mammals, 773*, 776*, 778*, 822*)—488, 565, 672, 674, 678, 773, 766, 768, 776, 777, 778, 822, 885, 887, 7877, 9687, 42664, 42666, 46668, 46787; Actinomyces_israelii (a bacterium normally found in the bowel and throat that can cause deep, pus filled holes in tissue. Also see Streptothrix, 222*, 262*, 488*, 567*, 7880*)—23, 222, 262, 2154, 465, 488, 567, 7880, 10000, 787, 747, 727, 20, 660, 690. 160, 73, 1.1; Actinomycosis (use Streptothrix); Acupuncture_disturbance_field (scar focus)—5.9; Acute pain—3000, 95, 10000, 1550, 802, 880, 787, 727, 690, 666; Adenoids—1550, 802. 880, 787, 776, 727, 444, 20, 428, 660, 2720, 2170, 14, 333, 523, 768, 786, 428, 1865, 590, 690, 780, 807, 810, 1570, 2000; Adenoma_cervical (epithelial tumor of the cervix that can be either benign or malignant Also see Cancer.)—433; Adenovirus (a virus that can cause colds/flu and infections in the lungs, stomach, and intestines)—333, 523, 666, 786, 768, 959, 962; Adenovirus_36 (AD-36 may be related to weight gain in some cases)—8875, 6140, 5859, 5797, 5219; Adenovirus_comp—10000, 7767, 7762, 7702, 7009, 7001, 6989, 5000, 4868, 2720, 2050, 1500, 1395, 1062, 1060, 1034, 1009. 969, 968, 962, 959, 960. 952, 951, 942, 880, 1550, 802, 787, 768, 728, 690, 660, 555, 523, 522, 1865, 444, 333, 300, 180, 160, 125, 95, 72, 60, 48, 26, 20; Adenovirus_HC—28729.05, 16628.88, 18471.00, 18670.15, 19566.32, 1407.93, 827.90, 919.62, 929.53, 974.15; Adhesions—2720, 2170, 1550, 802, 880, 787, 776, 760, 727, 660, 190; Adnexitis (swelling of the ovaries or Fallopian tubes)—440, 441, 522, 572, 3343, 3833, 5312; Adrenal_stimulant—10, 20, 2250; Adynantia_geriatric (fatigue of age)—410, 220, 100, 60, 27.5; Aflatoxin (a liver-damaging toxin produced by certain food molds)—344, 510, 943, 474, 476, 568; Aflatoxin_HC—438.74, 466.01, 9359.97, 8812.31; African_trypanosomiasis—656, 988, 780; Agyfla_HC—3534.88, 175.99; AIDS_1 (not for use in U.S. see HIV)—2489, 465, 727, 787, 880, 1550, 1500, 1.2, 31000, 31750, 34750; AIDS_2—1.44, 1550, 1500, 249, 418, 727, 787, 880, 2489, 3100, 3175, 3475; AIDS_Kaposis_sarcoma (use Cancer_Kaposis_sarcoma); AIDS_secondary (use 1113 for 12 min, 2128 for 16 min 6121 for 21 min, 33 for 1 min)—1113, 2128, 6121, 33; Akathisia—230, 7.83, 3; Alcoholism—10000; Allergy (also see Pertussis)—10000, 7:44, 5000, 1550, 1234, 740, 880, 835, 787, 727, 500, 330, 160, 30, 3; Allergy_1—10000, 1865, 880, 787, 727, 690, 660, 555, 522, 473, 444, 330, 290, 146, 125, 95, 72, 33, 20, 3; Allergy_pollen_TR (pulse 64 75, converge 2 0.03125, dwell 500)—116113, 119061.03, 119441.8; Alopecia (loss of hair)—10000, 30000, 5000, 2720, 2170, 1552, 880, 800, 787, 727, 465, 330, 146, 95, 28, 20, 3; Alpha_streptococcus_HC—18919,09, 18670,15, 941,93, 929.53; ALS_1 (Anwotropic lateral sclerosis, possibly caused by mycoplasma fermentans: Also use MS and see Echo virus, Coxsackie, Herpes 6, Bartonella, and Lyme freqs. Use 2900 for 30 min)—2900, 864, 790, 690, 610, 470, 484, 986, 644, 254; ALS_2 (Potential causative viruses)—5000, 3636, 2632, 1850, 1500, 1488, 1422, 1189, 1044, 922, 868, 845, 822, 788, 776, 766, 742, 733, 721, 676, 654, 625, 620, 607, 608, 609, 610, 611, 612, 613, 595, 515, 487, 461, 435, 423, 380, 322, 283, 232, 144, 136, 20; ALS_3 (use 2900 for 60 min)—2900; ALS_4 (also use for MS)—10000, 5000, 2900, 2750, 2700, 2145, 938, 862, 841, 777, 766, 741, 739, 688, 682, 660, 572, 532, 520, 477, 442, 433, 344, 343, 342, 338, 324, 322, 253, 242, 112; ALS_5 (use 2900 for 20 min)—2900, 986, 864, 790, 728, 690, 660, 644, 980, 692, 610, 484, 254; Alternaria_tenuis (a fungus associated with lung ailments)—853, 304; Alzheimers_1 (also see ALS sets)—430, 620, 624, 840, 866, 5148, 2213, 19180.5, 742,4, 303, 2.3,2, 3773.3, 943.3, 471.66, 470.9 941.8, 3767.3;

Alzheimersi—19180.5, 2213, 5148, 866. 840, 624, 620, 4:30; Alzheimers_TR—430, 620, 624, 866, 5148, 2213, 1918, 742.4, 303, 23.2, 2900, 864, 790, 690, 610, 470, 484, 986, 644, 254, 30, 33, 6; Amenorrhea (absence of menstruation)—10000, 880, 1550, 802, 787, 760, 727, 465, 20: Amoeba (a single celled, sometimes infectious microorganism)—310, 333, 532, 732, 769, 827, 1522; Amoeba_hepar_abcess (liver abcess caused by amoebic infection) 344, 605, 510, 943; Amoebic_dysentery (use Entamoeba histolytica); Amyotropic_lateral_sclerosis (use ALS); Anal_itching (also use Parasites enterobiasis and see Parasites general set)—10000, 880, 787, 760, 727, 465, 125, 120, 95, 72, 444, 1865, 20, 773, 826, 827, 835, 4152; Anaphylaxis—10000; Anaplasma_marginale_HC—19267.60, 959,28, 21010.14, 1046.03; Aneurysm—880, 787, 760, 727, 465, 125, 95, 72, 444, 1865, 20, 727; Angina_quinsy (inflammation of lymph gland in throat)—787, 776, 727, 690, 465, 428, 660; Angina_pectoris—3, 230, 2720, 2170, 1800, 1600, 1500, 880, 832, 787, 776, 727, 465, 444, 1865, 125, 95, 72, 20, 660, 783; Ankylosing_spondylitis (arthritis of the spine. Use 326 longer periods after buildup)—3000, 95, 1550, 802, 880, 787, 776, 727, 650, 625, 600, 28, 10, 35, 28, 7.69, 1.2, 110, 100, 60, 428, 680, 326; Anosmia (loss of smell)—20, 10000; Anthrax (also see Bacillus-anthracis_HC)—4000, 16655, 1370, 1365, 930, 900, 768, 633, 500, 420, 414, 400, 273, 224, 129; Anthrax_1—644, 643, 642, 639, 638, 637, 634, 633, 632, 629, 628, 627, 624, 623, 622; Antiseptic (use General antiseptic); Anxiety_1 (General anxiety disorder. Use 1.5 for 3 min, 6.8, 7.8 for 5 min, 95 for 3 min, 10000 for 5 min)—1.5, 6.8, 7.8, 95, 10000; Apoplexy_stroke_paralysis—40, 1800, 880, 787, 727, 650, 625, 600, 125, 95, 72, 20, 1865, 522, 428; Appendicitis—1550, 802, 880, 787, 727, 444, 380, 190. 10, 650, 444, 522, 125, 95, 72, 20, 522, 146, 440, 450; Appendicitis_1—1770, 1570, 880, 807, 804, 1550, 802, 787, 728, 690, 660, 622, 650, 625, 600, 450, 1865, 444, 440, 380, 190, 146, 125, 522, 95, 72, 20, 10; Appetite_lack_of—465, 444, 1865, 125, 95, 72, 20, 10000, 880, 787, 727; Arenas_tennus13 667; Arnica (a topical healing herb)—1042, 1032; Arryhthmia_heart_TR (also use magnesium, CoQ10, and fish oil)—696, 7.83; Arsenic_alb (homeopathic cell salt)—562; Arteriosclerosis (hardening of the arteries; regeneration takes time. Also try nanobacteria, coxsackie, and CMV freqs)—10000, 2720, 2170, 1800, 1600, 1500, 880, 787, 776, 727, 20; Arthritis_A—1500, 727, 784, 880; Arthritis_arthralgia_due_to_gout 9.39, Arthritis (arthrosis and parathyroid disturbances affecting calcium metabolism)—9.6; Arthritis (focal origin gastrogenic tonsiltogenic and paresis)—9.39, Arthritis_general (use Strep pneumonia and Mycoplasma general if needed)—10000, 5000, 2720, 1664, 1550, 1500, 962, 880, 802, 800, 787, 776, 766, 727, 688, 683, 650, 625, 600, 120, 20, Arthritis_1 (use 1.5 for 10 min, 120 for 20 min)—10000, 5000, 3176, 3000, 2720, 1664, 1550, 1500, 880, 802, 787, 770, 728, 690, 660, 512, 250, 230, 120, 100, 80, 60, 40, 30, 28, 26, 25, 20, 10, 9.4, 9.39, 7.7, 7.69, 3, 1.5, 1.2; Arthritis_rheumatoid (Cause could be bacteria like strep prieumoniae, chlamydia pneumonia, mycoplasina fermentans, or dental bacteria which must be addressed too. Also try General antiseptic and Parasites roundworms general if no response. Use 766 longer periods after building up.)—250, 1,2, 650, 625, 600, 787, 727, 262, 776, 766; Arthritis_secondary—2720, 1000, 1500, 770; Asian_grippe_A—516, 656, 434; Aspergillus_flavus (mold found on corn, peanuts, and grain that produce aflatoxin)—1823, 247, 1972; Aspergillus_general—1972, 1823, 758, 743, 697, 524, 374, 339, 247; Aspergillus_glaucus (blue mold occurring in some human infectious processes)—524, 758; Aspergillus_niger (common mold that may produce severe and persistent infection)—:374, 697: Aspergillus_terreus (mold occasionally associated with infection of the bronchi and lungs)—743, 339; Asthma (also see Liver support and Parasites ascaris and use: Mycoplasma general if needed)—7344, 3702, 3672, 2720, 2170, 1800, 1600, 1500, 1283, 1234, 1233, 880, 787, 727, 522, 444, 146, 125, 95, 72, 20, 0.5; Asthma_1—1283, 1233.4.7; Asthma_2 (all frequencies for 5 min)—1234, 3672, 7346, 727, 787, 880, 10000, 47, 120; Asthma_y—3125, 3124, 890, 886, 871, 822, 782, 756, 712, 665, 633, 521, 515, 487, 434, 411, 322, 263, 172, 128; Astrocytoma (use Cancer, astrocytoma); Ataxia (incoordination of muscles. Slow results in some cases)—2720, 2170, 1800, 1600, 1500, 880, 787, 776, 727, 650, 625, 600, 444, 1865, 125, 95, 72, 20; Ataxia_spastic—9.19, 8.25, 7.69; Atherosclerosis (commonly present microbes in plaque are herpes zoster and Chlamydia pneumonia, this set combines the two. Also use magnesium and vitamin C to help reduce mineral deposits)—7543.4, 7520.5, 4710.5, 3773.3, 3760.3, 1886, 1880, 943.3, 940, 620, 479, 471,66, 470,9, 941.8, 3767,3, 7160, 3343, 2431, 2323, 1577, 1544, 40887, 958, 934, 787, 786, 738, 718, 716, 686, 668, 643, 576, 574, 573, 572, 563, 554, 542, 453, 446, 436, 425, 423, 411, 345, 333, 223, 134; Athletes_foot (also see Epidermophyton floccinum, Tinea, and Trichophyton rubrum freqs. Use all freqs for 5 min)—20, 379, 727, 787, 880, 5000, 644, 766, 464, 802, 1552, 9999, 752, 923, 3176, 304; Atmenic_Alb—562; Attention_Deficit_Disorder (Probably important to avoid preservatives, aspartame, dyes, MSG, and other potential toxins, plus limit sugar and starches but ensure adequate protein and good oils in the diet. Try Shigella, Chlamydia pneum., and General antiseptic); Autointoxication—522, 146, 1550, 10000, 800, 880, 787, 727, 20;

Babesia—76, 570, 1583, 1584, 432, 753, 5776; Bacillinum (homeopathic nosode)—132, 423, 432, 785, 853, 854, 921, 1027, 1042, 1932; Bacillus_anthracis_HC—19665.89, 18122.49, 18321.64, 19317.38, 961.76, 912.18, 902.27, 979.11; Bacillus_cereus—18645.26, 928.29; Bacillus_Coli_Rod_Form—8020, 800, 683, 417, 318, Bacillus_Coli_Virus—17220, 11103, 8581, 2872, 1722, 1552, 770; Bacillus_infections (B. coli, B. coli rod)—787, 880, 802, 727, 1552, 800, Bacillus_lichenoformis—2655, 21554; Bacillus_subtilis (can cause conjunctivitis)—432, 722. 822, 1246; Bacillus_subtilis_nigerHC—19168.02, 18919.09, 18670.15, 954.32, 941.93, 929.53; Bacillus_thuringiensis—520, 2551, 902, 1405; Backache _1 (if no relief from these, use kidney stimulation freqs, magnesium and B6, and drink plenty of water. Use 212 and 305 for 6 min and 33 for 5 min)—10000, 1550, 880, 802, 787, 760, 727, 305, 212, 41.2, 33; Backache_2—787, 784, 776, 728, 727, 465, 432, 9.6, 9.4, 9.3, 7.7, 7.6, 3, 0.5; Backach_and_spasms_1—120, 212, 240, 424, 465, 528, 760, 727, 787, 880, 1550, 2112, 5000, 10000; Backache_chronic_lower 1 (use with back toward tube. Use 728 for 30 min)—728; Bacteria_lactis_nosode—512, 526, 798, 951, 5412; Bacterial_capsules_HC—20786.10, 17923.34, 1034.88, 892.35; Bacterial_infections_general (if bacterial infection is chronic and the type is accurately diagnosed and neither frequencies nor antibiotics are effective long term, also use Parasites general and roundworms sets. Also see General antiseptic and specific types.)—20, 465, 866, 664, 690, 727, 787, 832, 800, 880, 1550, 784; Bacterium_coli (a type of E. coli normally found in the intestines, water, milk, and soil that is the most frequent cause of urinary-tract infections and a common cause of wound infection)—642, 358, 539; Bacterium_coli_commune_combination—282, 333, 413. 957, 1320, 1722; Bacteroides_fragilis (use with Parasites ascaris set)—633, 634, 635, 636, 637; Bacteroides_fragilis_HC—16180.80 16230.58, 808.07, 805.59; Bad_breath (use Halitosis); Bakers_Yeast_allergy (homeopathy preparation)—775, 843, 1000; Balantidium_coli_HC (cysts. B. coli is the largest ciliated protozoon found in humans and can cause a severe colitis with ulcerations)—22902.05, 1140.23; Bantis_syndrome (Ailment in which blood vessels between the intestines and the liver become blocked, leading to congestion of the veins, an enlarged spleen, bleeding of the stomach and intestines, cirrhosis of the liver, and blood cell destruction)—1778; Barley_smut (homeopathic preparation for an allergen)—377, 224, 1447; Barretts_esophagus_1 (changes in esophageal cells usually caused by acid reflux. use 2127,5 for 6 min)—10000, 880, 802, 787, 776, 676, 727, 20, 146, 2127.5; Bartonella_henslae (virus which causes cat scratch fever)—364, 379, 645, 654, 786, 840, 842, 844, 846, 848, 850, 857, 967, 6878, 634, 696, 716, 1518; Basidiomycetes—751; BC_G_Vaccine—277, 768, 832, 725; Bed_wetting (enuresis. See also Parasite general, pinworm, and ascaris freqs)—1550, 880, 802, 787, 727, 465, 120, 112, 7.83; Bedsores—880, 1550, 802, 787, 784, 728, 727, 465, 20, 1.2, 73; Bells_Palsy_1 (also see Herpes_general)—2.4, 3, 3.9, 7.83, 20, 27.57, 33, 35, 40, 47.5, 57.5, 72, 90.88, 110, 125, 194, 222, 304, 393.5, 464, 565.5, 600, 625, 650, 727, 776, 787, 833, 880, 932.5, 1250; Bermuda_smut (homeopathic preparation for an allergen)—422, 767, 847, 971, 644, 780; Besnoitia_HC (protozoan taken from lung sect.)—17823.77, 887.39; Beta_streptococcus_HC—19168.02, 954.32; Bile_duct_1 (spasms and pain)—2, 2.2, 2.5, 2.7, 3.2, 3.5; Biliary_cirrhosis (an inflammatory condition in which bile flow through the liver is obstructed)—381, 514, 677, 2271, 1250, 170, 715, 774, 776; Biliary_headache—8,5, 3.5; Biliousness—1550, 802, 10000, 880, 832, 787, 727, 465; Bilirubin (a bile pigment that may result in jaundice in high concentrations. See also Liver support)—717. 726, 731, 863, 9305, 649, 734, Black_widow_spider—376, 728; Bladder_and_prostate_complaints—2050, 880, 1550, 802, 787, 727, 465, 20, 9.39; Bladder_TBC—642, 771, 360, 726, 724; Blastocystis_hominis (595*, 13469*)—365, 595, 844, 848, 1201, 1243, 5777, 11425, 11841, 11967, 13145, 13469, 21776; Blepharisma—3120; Blepharisma_HC—20238.44, 1007.61; Blood_diseases—880, 787, 727; Blood_pressure_high (use Hypertension), Blood_pressure_low (use Hypotension); Blue_cohosh (a healing herb)—364; Boils (use Funinkulosis); Bone disease_and periodontal disease (see Osteo freq. Use 728 for 15 min. combine with adequate nutrition, especially protein and essential fatty acids, and minerals, especially calcium and magnesium)—47.5, 1800, 1600, 650, 625, 600, 880, 787, 776, 728; Bone_regeneration (combine with adequate nutrition, especially protein, and minerals, especially calcium and magnesium)—2720, 10000; Bone_spurs (also use mineral supplement plus high dose bromelain and water)—1.2, 120, 250; Bone_trauma (cuts, fractures)—380, 1550, 802, 10000, 880, 787, 727, 2720; Bordertella_pertussis_HC—16479.52, 820.47; Borrelia (use Lyme disease); Borrelia_burgdorferi_HC (causes Lyme disease, see also Lyme_disease)—18919.09, 941.93; Botrytis—1545; Botrytis_cinereas—1132, 212; Botulinum (a bacillus that causes an often fatal form of food poisoning)—518, 533, 639, 172. 1372, 691, 683, 253, 435, 668, 775, 2688, 660, 690, 727, 802, 1550, 831, 1552, 10000; Brachial_neuralgia—0.5; Brain_beta_stim_TR (7.83 run at duty 5, 22 and 19.5 run at duty 30 and pulse 7.83 50)—7.83, 22, 19.5; Brain_tumor (use specified type: Cancer, droglioma, astrocytoma, and or glioma); Branhamella_Moraxella_catarrhalis—2013, 579, 581, 687, 770, 772, 775, 778, 2013; Branhamella_Neisseria_catarrhalis_HC—19715.68, 981.59, Breast_fibroid_cysts (also use black walnut hull capsules and mixed tocopherols if possible)—1550, 1384, 880, 802, 787, 690, 666, 267; Breast_tumor_benign_TR (also include sweep from 19:351.563 to 10406.25 by 0.03125 dwell 1)—174, 482, 785, 1132, 5311, 3965, 405, 633, 191,178, 731, 6646, 178, 739.8, 2959.4. 1234, 3702, 3672, 7344, 7760, 10:357, 10380; Breathing_deep—1234, 3702, 3672, 7344, 7760; Brights_syndrome (use Nephritis); Bronchial_asthma (use Asthma), Bronchial_pneumonia (use Pneumonia, bronchial); Bronchiectasis (chronic dilatation of the bronchi)—342, 510, 778, Bronchitis—7344, 3672, 1234, 880, 743, 727, 683, 464, 452, 333, 72, 20, 9.39, 9,35; Bronchitis_secondary—776, 766, 688; Bronchopneumonia_borinum (use Pneumonia, bronchial); Brown_recluse_spider bite (also use a lot of Echinacea and vitamin C)—724, 884, 1830, 3260, 30008; Brucella _abortus (undulant fever or Bang's bacillus, found in cattle)—1423; Bnicella_pelitensis (form of Brucella found in goats and sheep)—748, 643, 695; Bruises—9.1, 110, 10000; Bubonic plague (use Versenia pestis); Bubonic_plague_secondary_infections—880, 787, 727, 20; Bunion_pain—20; Burns—190, 10000, 880, 787, 727, 465, 200; Bursitis (May be caused by any number of organisms and viruses; experiment with arthritis frequencies as well.)—880, 787, 727; BX _Virus (use Cancer_BX_virus); BY_Virus (use Cancer_BY_virus); Caeliacia—674; Calcifications—326; Calming—6000; Campylobacter (bacteria causing sudden infectious diarrhea in newborns, 732*, 1633*, 333*)—732, 733, 1633, 1834, 2222, 786, 768, 523, 333, 378, 705.86, 2823.5; Campylobacter_fetus_HC (smear)—18321.64, 912.18; Campylobacter_pyloridid_HC—17674.41, 879.96; Cancer (Basic comprehensive set. A complete set like this, or rotation of the general cancer sets, or Rife Technology's intensive carcinoma and sarcoma banks are used as Basic sets. Sets listed below for specific types are to be used in addition to basic sets. Also add frequencies determined to be beneficial from scans. Increase run times on frequencies thought to be most effective as detox allows. Don Tunney now includes E_coli_1 frequencies with Resonant Light's intensive regimens. Also use 11780000 Hz if device is capable. James Bare states that 10025 may be master frequency for cancer in general, 10026 for sarcoma. Use + and −3 Hz sweep about 10025, building up to longer periods like 5 min per freq, 2128*)—10000, 11780, 21275, 17034, 11430, 10025, 6766, 6064, 5000, 3713, 3176, 3040, 2950, 2876, 2790, 2720, 2452, 2189, 2182, 2128, 2127, 2084, 2048, 2008, 1604, 1552, 1489, 880, 854, 800, 784, 776, 766, 728, 690, 683, 676, 666, 524, 464, 333, 120, 20; Cancer_adenocarcinoma—47, 2182, 2219, 832, 2084, 2127, 2160, 2452, 2876; Cancer_Adenoma—433; Cancer_Astrocytoma (common tumor of brain and central nervous system)—857, 9.19, 8.25, 7.69, 2170, 543, 641, 2127, 880, 690, 666; Cancer_bladder_secondary (use Parasites, schistosoma); Cancer_bladder_TBC (transitional bladder carcinoma)—642, 771, 360, 726, 724; Cancer_breast—3072, 2950, 2876, 2191, 2189, 2187, 2184, 2182, 2152, 2128, 2127, 2120, 2116, 2112, 2104, 2100, 2008, 1550, 866, 802, 732, 676, 666, 166, 120; Cancer_breast_1—3672, 2008, 2063, 2103, 2128, 2146, 2133, 2162, 2173, 2180, 2189, 2208, 2263, 2289, 2333, 1865, 444, 125, 95, 72, 48; Cancer_breast_2—656, 127, 1582, 478, 982, 2134, 2120, 9000, 9999, 304; Cancer_breast_3 (use 2128 for 56 mini—2128, 33, 1131; Cancer_breast_secondary—422, 942, 4412, 1862, 808, 1552, 728, 2720, 1234, 690, 2160, 2136, 477, 28, 317, 96, 3176, 3040, 2145, 2048, 1830, 2112; Cancer_BX_virus (carcinoma virus. Sweep up and down 1000 hz on MHz freqs and use 0.01 Hz sweep on 2876 and 2790, 2128*, 11780000*)—11780000, 17033662, 1604368, 21275, 17034, 46015.6, 23007.8, 11503.9, 10025, 3713, 2876, 2790, 2128, 2008, 1604; Cancer_BX2_TR (also use 21275, 20080, 17220, 1604000, 11780000 if device capable)—5318.8, 8610, 8020, 5278.3, 1675, 5020, 2128, 2127.5, 2127, 2663, 334, 2655, 5388.5, 2385, 6687.3, 3324, 8836.9, 2521, 7356, 2787.5, 5575, 8368.2, 1566.4, 2008, 5013, 5013.5, 10025, 10026, 10027, 7037.5, 263.11; Cancer_BY_virus (sarcoma virus, also use Cancer_sarcoma)—11430000, 20080, 17034, 11780, 11430, 3524, 2128, 2008; Cancer_carcinoma_basal_cell_skin (use 2116 for 30 min, others for 5 min, 2116*)—2116, 760, 2128, 2280, 2876; Cancer_carcinoma_basal_cell_skin_1 (use with 5 Hz fuzz)—11546700, 11546730, 11546750, 11546760, 11546870, 11546900; Cancer_carcinoma_bronchial—462, 776, 852, 1582, 2104, 2144, 2184, 3672; Cancer_carcinoma_colon (656*)—656; Cancer_carcinoma_general—2128, 1820: Cancer_carcinoma_larynx—327, 524, 7311, 1133; Cancer_carcinoma_liver_1—393, 479, 520, 734, 3130; Cancer_carcinoma_liver_fermentative 214: Cancer_carcinoma_liver_secondary (use Hepatitis B and Parasites, fluke, liver); Cancer_carcinoma_original_crane—21275, 2127.5; Cancer_carcinoma_scan—728, 690, 2008, 2104, 2112, 2120, 2128, 2136, 2144, 2152, 2160, 2168, 2176, 2184, 2192, 2200, 22117, 5000, 9999, 304; Cancer_carcinoma_uterine_fermentative—127; Cancer_cells_conidium_head—728; Cancer_cetvical_secondary (use Papilloma and Herpes viruses); Cancer_droglioma (see also Cancer, glioma, astrocytoma)—853; Cancer_experimental_additional_frequencies—55,56, 6.8, 66.5, 440, 778, 1050, 1550, 2180, 663, 3672; Cancer_fibrosarcoma (malignancy containing connective tissue and developing rapidly from small bumps on the skin)—1744; Cancer_fibrous_tumor_secondary—1340; Cancer_gastric_adenocarcinoma—676; Cancer_general_1—10000, 5000, 3176, 2720, 2489, 2189, 2184, 2128, 2084, 2050, 2008, 880, 854, 800, 784, 728, 666, 524, 464, 333, 304, 120; Cancer_general_2—10000, 3176, 3040, 2720, 2489, 2182, 2127, 2048, 2008, 1862, 1552, 880, 802, 786, 727, 665, 664 465, 304, 125, 96, 72, 64, 20; Cancer_general_3—10000, 3176, 2950, 2180, 2128, 2049, 2008, 1865, 1488, 943, 886, 866, 776, 732, 728, 690, 676, 650, 523, 442, 414, 304, 240, 128; Cancer_glioblastoma—720, 2008, 2128, 2180, 2182, 728, 832, 800, 664, 20, 855, 543, 641, 857; Cancer_glioblastoma_tumor—463, 466, 470; Cancer_gliomas (largest group of brain cancers)—543, 641, 857; Cancer_harmonic_series (may be based on original Rife/Hoyland cancer frequencies. Extend harmonic series with 66.5 as base, up to 10,000, in this manner: Base*1, 2, 3, 4, 5, 6, 7, 8, 9, 10, etc)—66.5, 133, 199.5, 266, 332.5, 399, 465.5, 532, 598.5, 665: Cancer_Hodgkins_disease (a form of malignancy characterized by enlargement of the lymph nodes, spleen, and lymph tissue and often includes weight loss, fever, night sweats, and anemia. Also called lymphogranuloma. Also see Chlamydia pneumoniae, Pseudomonas, Penicillium, Aspergillus, and Rhizopus.)—552, 1522; Cancer_hodgkins_TR—1552.552, 5318.8, 8610, 8020, 5278.3, 1675, 5020, 2127, 2663, 334, 2655, 5388.5, 2385, 6687.3, 3324, 8836.9, 2521, 7356, 2787.5, 5575, 8368.2, 1566.4, 2008, 5013, 5013.5, 10025, 10026, 10027, 7037.5, 263.11; Cancer_Kaposis_sarcoma (also use Herpes type 8)—249, 418, 647; Cancer_leukemia (also use Leukose)—2127, 2008, 880, 787, 727, 690, 666, 2217: Cancer_leukemia_feline (cat)—258, 332, 414, 424, 535, 536, 544, 830, 901, 918, 997, 741, 743, 1071.5; Cancer_leukemia_hairy_cell (typified by abnormal bbod cells and shortage of others)—122.622, 932, 5122, 488, 781: Cancer_leukemia_lymphatic—478.833: Cancer_leukemia_myeloid (characterized by rapid growth of incompletely formed while blood cells)—422, 822: Cancer_leukemia_T_cell—222, 262, 822, 3042, 3734; Cancer_leukemia_TR—2127, 2008, 880, 822, 787, 727, 690, 666, 590, 10000, 2008, 1850, 450, 440, 422, 428, 2030, 2030, 2030, 465, 15, 14, 6.8; Cancer_liver (use Cancer, carcinoma, liver); Cancer_liver_secondary (use Hepatitis B and Parasites, fluke, liver); Cancer_lung (use Cancer, carcinoma, bronchial); Cancer-lymphogranuloma_lymphoma (use Hodgkin's disease); Cancer_lymphogranuloma_venereum_secondary (use Chlamydia trachomatis); Cancer_lymphosarcoma—482 Cancer_maintenance_secondary—120, 250, 428, 465, 600, 625, 650, 661, 664, 667, 690, 728, 776, 784, 800, 802, 832, 880, 1489, 1550, 1600, 1865, 2000, 2012, 2100, 2170, 2490, 2730; Cancer_melanoma_1 10000, 2217, 2128, 2127, 2050, 2008, 1050, 1000, 880, 787, 728, 666, 495, 465, 450, 125, 100, 95, 80, 60, 45, 20, 10, 7.5; Cancer_melanoma_2—2217, 2128, 2050, 2008, 1552, 1102, 1050, 1000, 979, 907, 880, 802, 787, 728, 666, 495, 466, 465, 450, 125, 110, 100, 95, 80, 60, 45, 20, 10, 7.5; Cancer_melanoma_metastasis—979; Cancer_multiple_myeloma—21275, 11780, 5122, 4750, 4213, 2950, 2145, 2128, 2107, 2008, 1488, 822, 781, 647, 526, 475, 422, 418, 263, 249; Cancer_mycosis_fungoides (a form of skin cancer resembling eczema)—852; Cancer_nasopharyngeal_secondary (use EBV): Cancer_neuroblastoma (exp)—7027, 6148, 5270, 4392, 3513, 2635, 1757, 878; Cancer_non_Hodgkins_1 (also use Cancer, Melanoma)—574, 588.666, 778, 1078, 1120, 1340, 1744, 3524, 3713; Cancer_non_Hodgkins_2 (also use Simian_virus_40)—2008, 2004, 2012, 2116, 2128, 3672, 7760; Cancer_not_kiled_by_2008_2128—2180, 2182.21, 2184; Cancer_pain—3000, 95, 2127, 2008, 727, 690, 666; Cancer_pancreatic (use Cancer_adenocarcinoma); Cancer_plasmacytoma (a tumor with plasma cells that occurs in the bone marrow, as in multiple myrloma, or outside of the bone marrow, as in tumors of the inner organs and lining of the nose, mouth, and throat)—475; Cancer_prostatc (also see Prostate adenominum and Prostate hyperplasia)—20, 60, 72.95, 125, 666, 727, 787, 790, 766, 800, 920, 1998, 1875, 442, 2008, 2127, 2128, 2217, 2720, 2050, 2250, 5000, 2130, 2120, 690, 304. Cancer_prostate_1 (use all freqs for 6 min after assessing detox)—2128, 2125, 2131, 2140, 2145, 666, 672; Cancer_prostate_Vega_1—854, 1840, 2145, 2288; Cancer_rhabdomyosarcoma_1—2000, 2005, 2008, 2016, 2048, 2084, 2093, 6024, 2100, 2128, 2127, 2184, 2217, 6384, 728, 784, 880, 464; Cancer_rhabdomyosarcoma_embryonal (2586*)—6384, 6024, 2586, 2217, 2184, 2128, 2127, 2100, 2093, 2084, 2060, 2048, 2040, 2032, 2016, 2008, 2005, 2000, 880, 784, 728, 464: Cancer_rhabdomyosarcoma_embryonal_v—2586, 5476, 4445; Cancer_sarcoma_general (BY)—1755, 3524, 2007.5, 2005, 2015.9, 2083.8, 728, 880, 802, 785; Cancer_skin (use Cancer, carcinoma, basal cell skin and specific forms); Cancer_stomach_secondary (use Helicobacter pylori); Cancer_tertiary—20, 421, 965, 50, 383; Cancrum_oris (rapidly growing oral or nasal ulcer)—20, 727, 787, 802, 880; Candida (also see Parasite general, roundworm, and ascaris if these don't work long term. Some think that chronic candida cannot be cured unless toxic metal accumulation is reduced or eliminated by using a metals cleanse. 464*)—3176, 2644, 1403, 1151, 943, 886, 877, 866, 762, 742, 661, 465, 464, 450, 414, 412, 386, 381, 254,2, 120, 95, 64, 20; Candida_1—10000, 50000, 3176, 2489, 1395, 1276, 1160, 1044, 928, 877, 812, 728, 696, 580, 465, 464, 381, 348, 232, 116, 58, 20; Candida_2 (includes candida carcinomas and tropicalis)—1403, 675, 709, 2167, 2128, 2182, 465, 20, 60, 95, 125, 225, 427, 464, 727; Candida_albicans_HC—19217.81, 956.80; Candida_carcinomas—2167, 2128, 2182, 465; Candida_secondary (also use other parasite sets esp roundworm frogs if necessary)—72, 422, 582, 727, 787, 802, 1016, 1134, 1153, 1550, 2222, 412, 543, 2128; Candida_sweep_TR (sweep from 72006.25 to 12137.5 by 0.03125 dwell 0.5 pulse 64 75); Candida_tertiary (some causal factors)—880, 95, 125, 20, 60, 225, 427, 240, 650, 688, 152, 442, 8146, 751, 1146; Candida_tropicalis—1403, 675, 709; Canine_Parvovirus—185, 188, 323, 428, 433, 562, 613, 622, 1000, 4027; Canine_parvovirus_mutant_strain—323, 514; Canine_parvovinis_type_B—323, 535, 613, 755, 761, 764, 766, 768; Canker_sore (use Stomatitis aphthous); Carbo_animalis (homeopathic remedy from animal bone charcoal)—444; Carbuncles (use Staphylococcus aureus); Cardiacedema—9.19; Carpal_tunnel_secondary (also use Arthritis)—2008, 666; Carvularia_spitatera—879; Cat_scratch_fever (use Bartonella henslae); Cataract (also use eye formula antioxidant supplement for all macular degeneration problems, use 1830 for 10 min, 1830*)—1830, 728, 784, 787, 800, 880, 10000; Cataract_1—1830, 1600, 9999, 1552, 2110, 1335, 1654, 2187, 2195, 2211; Cataract_2—292, 30, 9.1, 0.3; Cataract_brunescent (brown opacity in later life, 1830*)—2010, 1335, 1830; Cataract_complicated (secondary type caused by disease, degeneration, or surgery, 1830*)—1830, 496, 325, 774; Cataract_general (run 1830 extended period, 1830*)—10000, 2211, 2195, 2187, 2110, 2010, 1830, 1654, 1600, 1552, 1335, 1335, 880, 800, 787, 784, 774, 728, 496, 325; Catarrh—1800, 1713, 1550, 802, 800, 880, 787, 727, 444, 20; Causticum—540, 1013; Celiac_disease_1—39, 2, 3, 4.18, 5.13; Cells_of_Leudig (colon tonic)—2500; Cephalosporium (fungi that are the source of some broad spectrum antibiotics)—418, 3966, 544; Cephalothecium—371, 574, 6933; Cerebral_palsy—880, 787, 727, 522, 146; Cerebrospinal_troubles—10000; Cerumen (ear wax)—311, 320, 750, 984, 720; Cervical_polyp—277, 288, 867, 687, 744; Cervicitis (womb neck inflammations)—20, 727, 787, 880; Cervix adenoma (epithelial tumor of the cervix)—433; Chaetomium_globosum (867*) 221, 867, 102; Chalcra_base_root (pulse at 4 Hz)—20; Chancre (use Syphilis); Chelidonium—162; Chemical_sensitivity (see also Liver support)—727; Chemtrail_detox (can also be useful for lung and sinus problems, All frogs for 5 min)—664, 7344, 2842, 1147, 686.6, 684.1, 1113, 779.9, 829,3, 679.2, 865, 969,9, 11067, 78:3.6, 800.4, 1045, 1062, 673.9, 690.7; Chest_infection_secondary—72, 333, 452.683; Chickenpox (by the time pox marks appear, the illness is resolved and the toxins are being expelled through the skin, so must be used in the early stages of flu like symptoms after exposure. Use herpes zoster.); Chillbains (use Perniosis), Chilomastix_HC (amoeba cysts)—1936′7.117, 964.23, 21209.29, 1055.95; Chilomonas_HC—19815.25, 986.54; Chlamydia_general—3773, 3768, 222:3, 2218, 2213, 942, 866. 840, 622, 555, 470, 430; Chlamydia_pneumontae—7543.4, 7520.5, 4710.5, 3773.3, 3760.3, 1886, 1880, 943.3, 940, 620, 479, 471,66, 470.9, 9411.8, 3767,3, Chlamydia_trachomatis (a usually sexually transmitted bacterial infection causing trachoma, inclusion conjunctivitis, lymphogranuloma venereum, urethritis, and proctitis.)—430, 620, 624, 840, 1111.4, 2213, 866, 555.7, 2222.8; Chlamydia_trachomatis_HC—18968.87, 944.40; Cholecystitis_acute (painful gallstone attack)—481, 743, 865, 928; Cholecystitis_chronic (long-term inflammation of the gall bladder)—432, 1551, 801; Cholera (an extremely contagious and serious bacterial infection of the small intestines)—1035, 968, 961, 851 844, 843, 691, 591, 556, 330; Cholera_secondary—880, 802, 450, 832, 78′7, 727; Cholesteatoma (benign tumor usually found in middle ear and mastoid region)—453, 618, 793, 5058; Cholesterinum—1386, 173, 620, 635, 780; Chronic_fatigue_syndrome (use with General parasite, roundworm, and fluke sets if necessary. Also see EBV and Fatigue general sets. If no response to these, try Cancer leukemia hairy cell)—10000, 660, 2127, 787, 465, 424, 664, 120, 880, 1550; Chronic_Fatigue_v—1902, 1000, 959, 649, 568, 243, 922, 2422, 730, 1522, 116, 1489, 962, 172, 1333; Cimicifuga (plant family including black snakeroot and black cohosh)—334, 594; Circulation_disturbances—40, 9.39; Circulatory_stasis (stimulates blood circulation. See also Circulation disturbances)—40, 2112, 2145, 2720, 2489; Cirrhosis_biliary (an inflammatory condition in which bile flow through the liver is obstructed)—381, 514, 677, 2271; Cirrhosis_hepatitis—291; Cladosporium_fulvum (a pathogenic fungus)—438, 233, 776, 510; Clostridium_acetobutylicum_HC—19118.23, 951.84; Clostridium_{—-.}botulinumfIC (produces botulin toxin that causes botulism, a type of food poisoning)—18022,92, 897.31; Clostridium0—difficile (can cause diarrhea following application with antibiotics)—387, 635, 673; Clostridium_perringens_FIC (spores)—19715.68, 981.59; Clostridium_septicum_HC—18122.49, 902.27; CMV (use Cytomegalovirus); Coeliacia—154, 594, 656, 586, 668, 787, 7958, 665, 674; Cold_1 (use 800 and 880 for 10 min, others for 5 min)—5500, 4400, 802, 787, 727, 720, 552, 440, 400, 125, 72, 800, 880; Cold_2—652, 725, 746, 751, 768, 1110, 333, 666, 542, 522; Cold_3 (fail, 99. Use all &cgs 5 min)—20 120, 146, 440, 444, 465, 727, 776, 787, 880, 1500, 1550, 5000, 1000; Cold_4 (use 880 and 800 for 10 min, 728 for 5 min)—3176, 2489, 880, 800, 728; Cold_5 (use 7728 and 4888 for 10 min)—7728, 4888, 8238, 2413, 880, 787, 776, 727, 440, 746, 567, 7880, 787, 300, 310, 1234, 9999; Cold_6—7660, 7344, 5000, 3702, 3672, 2688, 2400, 1862, 1550, 1238, 1234, 1200, 975, 880, 802, 787, 780, 778, 776, 774, 772, 770, 768, 766, 727, 688, 683, 660, 450, 412, 352; Cold_and_Flu (fall, 98. Use 8700 and 7760 for 15 min, others for 5 min)—250, 465, 8210, 8700, 7760; Cold_and flu_winter—959, 962; Cold_in_head_chest (Mutates constantly; too many strains to include complete list of frequencies. See also Strep Pneumonia, Adenovirus, Rhinitis, Coronavirus, Sinusitis, Pneumonia, Chest infection, and Rhino pneumonitis sets. Use lots of echinacea at onset to prevent cell damage that prolongs healing, even if correct freqs are found.)—10000, 7344, 4412, 3176, 2489, 1550, 880, 802, 787, 776, 766, 728, 712, 688, 683, 665, 660, 600, 444, 333, 20; Cold_2005_TR—10000, 7344, 5000, 2950, 2900, 2650, 2600, 1550, 1234, 740, 880, 787, 727, 330, 165, 82.6, 41.75, 20.87, 30; Cold_sores (use Herpes Simplex I); Colic—1550, 832, 802, 787, 727, 20; Colitis_and_Diarrhea (inflammation of the colon)—10000, 5000, 1550, 880, 832, 802, 787, 727, 621, 465, 454, 440, 433, 344, 152; Collectotrichum—1482, Colon_problems_general—20, 440, 880, 1552, 802, 832; Colors (color decode from Jade Machine)—2155, 2055, 960, 920, 858, 815, 800, 745, 677, 640, 624, 470; Comedones (blackhead)—778; Complete_early_crane—34750, 31750, 31000, 10000, 5000, 3500, 2720, 2489, 2452, 2250, 2200, 2180, 2130, 2127.5, 2120, 2100, 2093, 2050, 2008, 2000, 1998, 1865, 1862, 1850, 1840, 1800, 1600, 1570, 1560, 1552, 1550, 1500, 1100, 1050, 1027, 1000, 885, 880, 875, 864, 804, 803, 802, 800, 799, 787, 784, 781, 780, 776, 775, 770, 764, 740, 730, 727.5, 725.712, 700 690, 676, 666, 664, 660, 650, 625, 622, 600, 500, 465, 450, 440, 432, 428, 423, 400, 304, 250, 240,162, 160, 125, 121, 120, 100, 95, 82, 81, 80, 78, 72, 70, 60, 41. 36, 30, 28, 26, 22, 20, 18,15, 14, 13, 10 9, 8, 7, 5.5, 5, 4. 3.5, 1.8, 1; Complete_early_rife—10000, 5000, 3500, 2489, 2250, 2127.5, 2093, 2008, 2000, 1865, 1850, 1800, 1600, 1570, 1560, 1550, 1500, 880, 802, 800, 787, 784, 776, 740, 727.5, 660, 650, 625, 622, 600, 500, 465, 450, 400, 250, 125, 120, 100, 80, 60, 20; Condylomata (usually venereal warts, caused by papilloma virus. Occur near intersection of mucous membranes and skin. Use also Papilloma.)—466; Conjunctivitis (also known as pink eye. Also use Chlamydia trachomatis and see Bacillus subtilis if necessary)—2025, 1830, 1552, 1550, 1246, 1206, 880, 822, 802, 787, 727, 722, 489, 432, 80, 20; Constipation (also use Parasites general and roundworm sets if necessary)—3176, 1550, 880, 832, 802, 787, 776, 727, 444, 440, 422, 20; Contusion (bruise)—9.1, 110, 2720; Convoforce—774; Convulsions_1—10000, 880, 787, 727; Convulsions_spasticity—9,19, 8.25, 7.69; Coraforce—774; Corallinus—533; Corn_smut—546, 1642, 289; Coronavirus_SARS—9918, 9740, 4959, 4870, 2479.5, 2435, 1394.7, 1369.6, 1239.7, 1217,5, 774.8, 760.9, 619,9, 608.7, 464.9, 456.5, 309.9, 304,4, 155, 152.2; Corynebacterium_diptheriae (use Diptheria); Corynebacterium_diptheriae_HC—17027.18, 847.73; Corynebacterium_xerosis_HC—15732.71, 783.29; Costalgia (rib pain)—10000, 880, 787, 727; Coughing—7760, 7344, 3702, 3672, 1550, 1500, 1234, 776, 766, 728, 720, 688, 683, 530, 525, 524, 522, 514, 444, 440, 432, 146, 125, 95, 72, 20, 7.7, 0.5; Cough_lingering_TR—4664.45, 3444.55, 2543.35, 1666.35, 3665.65; Coughing—453, 550, 674, 728, 1109, 7160, 634, 639, 1089, 522, 524, 525, 146, 1500, 1550, 0.5, 514, 530, 432, 440, 444, 720, 1234, 3702, 20, 125, 72, 95, 7.7; Coxsackie_B—353, 384, 834, 587, 723; Coxsackie_B1_HC—18122.49 902.27; Coxsackie_B2—705, 534, 867; Coxsackie_B3—612, 487, 868, 653, 654; Coxsackie_B4—421, 353, 540, 8632; Coxsackie_B4_HC—18047.81, 898.55; Coxsackie_B5—462, 1.043, 1083, 569, 647, 708, 774; Coxsackie_B6—488, 736, 814, 343, 551, 657, 668, 669; Coxsackie_General (see also Mumps)—612, 136, 144, 232, 380, 422, 424, 435, 921, 923, 769, 1189, 595, 676; Cramping_and_nausea—72, 95, 190, 880, 832, 787, 727, 20, 4,9; Cramps—10000, 880, 787, 727, 26; Cramps_menstrual—26; Crinis_humaasis—646; Crocus_sotillus—710; Crohns_and_other_bowelproblems_v—141, 488, 866, 468, 782, 664, 604, 187, 543, 1423, 972, 110, 133, 979, 173, 233, 672, 510, 447, 350; Crohns_disease (also use colitis, colon, and parasite freqs)—10000, 727,786, 440, 832, 880, 1550, 20; Crohns_disease_protozoa_1—200, 206, 249, 298; Crohns_disease_viroid_1—585, 593, 600; Croup (characterized by a loud cough that resembles the barking of a seal, difficulty breathing, and a grunting noise or wheezing during breathing)—10000, 7344, 3702, 3672, 1234, 1165, 580, 290, 870, 333, 666, 523, 786, 728, 880, 1550, 960, 712, 278, 683, 688, 776, 766, 444, 95, 72, 20; Cryptococcus_neoformans (yeast which causes respiratory infection than can turn into meningitis. Also known as torulosis.)—367, 428, 444, 476, 478, 522, 579, 594, 785, 792, 872, 2121, 5880, 5884, 597, 613, 624; Cryptosporidium (parasitic protozoa sometimes causing diarrhea in humans)—220, 482, 575, 4122, 698, 711, 893, 895, 1276, 5690; Cunninghamella—311, 323; Curva_spic—435; Cyclospora—543, 316, 992, 751, 268, 2144; Cyst_ovarian—982; Cyst_sebaceous_TR (streptothrox plus nocardia and actinomyces Israeli)—694, 719, 784, 228, 231, 237, 887, 2890, 222, 262, 2154, 465, 488, 567, 7880, 10000, 787, 747, 727, 20; Cyst_solitary—75, 76, 543; Cystic_fibrosis (Also called mucoviscidosis, a disorder of the exocrine glands that causes them to release very thick mucus. Use Psendomonas_aeruginosa, Breathing_deep, and General Antiseptic and see Parasites general and roundworm freqs)—523, 557, 478, 776, 660, 727, 778, 787, 802, 880; Cystitis_chronic (long-term inflammation of the urinary bladder and ureters)—246, 1550, 880, 802, 787, 727, 465, 20, Cystopyelo_nephritis (inflammation from bladder to kidney)—1385; Cysts_hydatid (use Parasites tapeworm), Cytochalasin_HC—4530.62, 3833.60, 225.57, 190.86; Cytomegalovirus (CMV, known as salivary gland virus or human herpes type 5. 2145*)—126, 597, 629, 682, 11045, 2146, 2144, 2145, 8848, 8856, Cytomegalovirus_HC—20362.91, 101:3.81, Cytophaga_rubra_HC—21408.44, 1065.86;

Deafness (partial to complete)—10000, 1550, 880, 802, 787, 727, 20; Deer_tick_1—7989, 289, 7:37, 738, 271, 671, 773; Dematium_nifrum (soil fungi found in human lesions)—24:3, 738; Dengue_fever_TR—160, 500, 1600, 5000, 7344, 4412, 1550, 1234, 740, 880, 787, 727, 330, 10000, 5000, 30, 10551.5, 5275.8, 2627.9; Dental_and_jawbone_infections_1 (use converge 1 1 on all except use converge 1 0.1 on 728)—7270, 2720, 2170, 880, 787, 727, 500, 190, 728; Dental_and_jawbone_infections_2—15, 326, 465, 727, 787, 880; Dental_foci (Neglecting this can prevent recovery from any illness if infection is a problem)—5170, 3000, 2720, 2489, 1800, 1600, 1550, 1500, 880, 832, 802, 787, 776, 727, 666, 650, 646, 600, 465, 190, 95, 47.5; Dental_general (see also Toothache)—728, 784, 635, 640, 1036, 1043, 1094, 685, 60, 48, 465; Dental_infection (roots and gums)—960, 660, 666, 690, 727, 784, 787, 800, 880, 1560, 1840, 1998, 2489; Dental_infection—5170, 3000, 2720, 2489, 1800, 1600, 1550, 1500, 1094, 1043, 1036, 880, 832, 802, 787, 776, 727, 685, 666, 650, 646, 640, 635, 600, 465, 190, 95, 60, 48, 47.5; Dental_infection_2—10000, 5000, 1562, 1552, 1550, 880, 800, 799, 787, 784, 776, 774, 728, 727, 664, 620, 464, 254.2, 120, 64, 20; Dental_infection_and_Earache_1—960, 930, 900, 880, 832, 802, 800, 787, 776, 775, 768, 760, 750, 727, 685, 680, 666, 650, 646.3, 646, 640, 635, 622,3, 547, 521, 518; Dental_infections_TR—3400, 2489, 1700, 1560; Dental_Infections_v—981, 138, 142, 177, 183, 210, 222, 233, 436, 534, 626, 723, 835, 875, 5227 7059; Depression_anxiety_trembling_weakness—3.5, 800; Depression_drugs_or_toxins—1.1, 73; Depressio_general (avoid starches in diet, use a multivitamin and multimineral, and drink plenty of water)—10000, 5000, 3176, 800, 787, 73, 35, 7.83, 3.5, 1.1; Detox_and_lymphs—10000, 3176, 3040, 880, 787, 751, 727, 676, 635, 625, 522, 465, 444, 440, 304, 306, 148, 146, 15,2, 15.05, 10,36, 10, 7.83, 6.3, 2.5, 3176; Detox_assist—10000, 3176; Detox_1—10000, 3176, 522, 164, 89, 24; Diabetes_1 (Warning; can cause large drop in blood sugar level. Use 5000 for 15 min)—5000, 2127, 2080, 2050, 2013, 2008, 2003, 2000, 1850, 880, 803, 800, 787, 727, 660, 484, 465, 440, 35, 20, 6.8; Diabetes_2—4200, 2128, 1865, 1850, 1550, 787, 465, 444, 125, 95, 72, 48, 302; Diabetes_associated_infection—2020, 800, 727, 786, 190, 80, 20; Diabetes_secondary—10000, 2720, 2170, 1800, 1550, 880, 802, 727, 465, 20; Diabetes_tertiary—1850, 32000, 4000, 500; Diabetic_loading—35, 700; Diabetic_toe_ulcer_1 (also use Staph aureus freqs and see Staph general, General antiseptic, and Circulation stimulation as needed)—786, 2112, 1050, 1.2, 5000, 832, 20; Diarrhea (see also Colitis, Clostridium difficile, E. coli, and for chronic problems, giardia and IBS freqs. Also see Parasite general set if no relief)—1550. 880, 832, 802, 786, 727, 465; Dientainoeba_fragilis_HC—20113.98, 1001.42; Diphtheria (bacterial infection causing sore throat, fever, rapid heartbeat, nausea, chills, headache and may progress to damage heart and nerves from bacterial toxins. Rare in US.)—10000, 1000, 925, 800, 788, 776, 624, 590, 432, 340, 151; Diplococcus_diptheriae_HC—17973.13, 894.83; Diplococcus_pneumoniae_HC—17923.34, 18172.28, 892.35, 904.74; Dirofilaria_immitis (use Parasites heartworm); Disc_herniated—727, 787, 2720, 10000; Distemper—242, 253, 254, 255, 312, 442, 551, 573, 624, 671, 712, 760, 940, 950, 1269, 1950. 8567. 660; Distortion (twisting of muscles, spine)—9.1, 110; Diverticulitis_acute (see Intestines, inflammation.All freqs for 10 min)—120, 500; Diverticulosis (characterized by tiny hernias of intestinal tissue protruding through the muscular wall of the colon)—154, 934; Dizziness (use Vertigo); DNA_repair (exp)—528, 731, 732; Dog_and_cat_hostility_1—3.6; Downs_syndrome_palliative—20; Droglionia (use Cancer, droglionia); Drug_addiction—20; Duodenal_ulcer (use Ulcer, duodenal); Duodenitis—223; Dupuytrens_contracture (4th and 5th finger curling into hand, unable to straighten)—1.2, 250; Dysentery (acute diarrhea with blood and mucus. Also use Entamoeba histolytica, Salmonella, and Shigella)—1552, 802, 832; Dysmenorrhea (painful menstruation. Use with plain water douche)—26, 4,9, 1550, 880, 802, 787, 727, 465; Dyspepsia (indigestion. if chronic or with bloating, also see Parasites general) -10000, 880, 1550, 832, 800, 787, 727, 465, 444, 20, 125, 95, 72, 4.9;

E_coli (Escherichia coli; can cause infections in wounds and the urinary tract. If using these leads to common cold symptoms, follow with Adenovirus freqs, 800/802*, 1550/1552*)—7849, 7847, 1730, 1722, 1552, 1550, 1320, 1244, 1000, 957, 934, 856, 840, 832, 804, 802, 800, 799, 776, 642, 634, 556, 548, 413, 333, 330, 327, 289, 282; (recommended for cancer adjunct)—7847, 1730, 1712, 1244, 1000, 934, 856, 840, 800, 776, 642, 634, 556, 539, 358, 330; E_coli_comp—7849, 7847, 1730, 1722, 1712, 1703, 1552, 1550, 1320, 1244, 1242, 1000, 957, 934. 856, 840, 832, 804, 802, 800, 799, 776. 642, 634, 632, 556, 548, 539, 413, 358, 333, 330. 327, 289, 282; E_coli_HC—17724.20, 19566.32, 974.15, 882,44; E_coli_mutant_strain—556, 934, 1242, 1244, 1703, 632, 634, 776; Ear_conditions_various (discharges, tinnitus, itching, hearing loss. Also see Otitis.)—10000, 880, 787, 727, 776, 766, 688, 683, 652, 645, 542, 535, 440, 410, 340, 201, 158, 20, 9.19; Ear_fungus_1—854; Ear_wax (use Cerumen) EBV (use Epstein-Barr Virus); Echinococcinum (use Parasites, tapeworms, echinococcinum); Echo_virus (Endometriosis Tuberylosa, can cause a type of viral meningitis)—922, 788, 765, 722, 625, 620, 614, 613, 612, 6111, 610, 609, 608, 607, 606, 605, 604, 603, 514.461; Eczema—9.119, 707, 1550, 802, 787, 727, 10000, 5000, 2720, 2008, 2180, 2128, 664, 1120, 20; Eczema_1—770, 916.415; Eczema_2—730.2, 1550, 802, 787, 690 Eczema_vascular_and_lung_disturbances—9.39: Edema (see also Kidney and Lymph stimulation)—40, 522, 146, 6.3, 148, 444, 440, 880, 787, 727, 465, 20, 5000, 10000; Electrolyte_levels (to improve)—8.1, 20, 10000; Electrosmog_1—99.5 Elephantiasis (use with parasite, esp roundworm and nematode freqs)—623, 824, 865, 710; Elkanella_corroderis_HC—19018.66, 946.88; Emotional_ties_to_diseases—764, 664; Emphysema (use with mycoplasma pulmonis, mycoplasma pneumonia, and parasite freqs, esp roundworm)—1234, 3672, 7344, 880, 787, 727, 120, 20, 80; Emphysema_comp—7344, 3672, 2838.5, 2688, 2404.2, 1234, 975, 880, 78′7, 777, 766, 727, 709.2, 688, 683, 660, 650, 601, 422, 300,5, 240, 150.3, 128, 120, 80, 75,1, 20; Encephalitis (inflammation of the tissues of the brain and spinal cord)—841; Endocarditis (use Listeriose); Endocrine_RX_TR (includes stimulating frequencies for pineal, hypothalamus, pituitary, thyroid, adrenal, and thymus gands)—662, 1725, 1342, 1534, 1413, 1351, 635, 763, 1335, 645, 10000; Endolimax_nana_FIC (trophozoites and cysts)—19715.68, 981.59, 21508.01, 1070.82; Endometriosis_1 (use 246, 800, and 1550 for 5 min)—6766, 6672, 6641, 6578, 2150, 2128, 2082, 2013, 2008, 2003, 2000, 1850, 1550, 945, 854. 846, 830, 800, 763, 676, 651, 524, 435, 275, 246, 142; Endometriosis_chronic (growth of uterine tissue outside the uterus that may cause pain, infertility, and abnormal uterine bleeding. Use with Parasites, flukes, general)—246, 800, 1550; Endometriosis_tuberylosa (use Echo virus); Energy_vitality—9999; Entamoeba_coil_HC (trophozoite)—19715.68, 981.59; Entamoeba_gingivalis_HC (trophozoite)—21806.74, 1085.69; Entamoeba_histolytica (highly damaging protozoa causing dysentery and liver infection)—148, 166, 308, 393, 631, 778; Entamoeba-histolytica_HC—19168.02, 954.32; Entamoeba_histolytica_secondaty—1552, 880, 802, 1550, 832, 787, 727, 690, 660, 465, 786, 768, 523, 333; Enterobacter_aerogenes_HC (causes intestinal problems)—18620.36, 927.05; Enterobiasis—(pinworms. Use Parasites, enterobiasis); Enterococcinum (homeopathic nosode for Strep family organisms found in the digestive and urinary tracts)—686, 409; Enterohepatitis (inflammation of bowel and liver)—552, 932, 953; Enterovirus_General (generic term meaning small virus, includes Echo, Coxsackie, and Polio)—5000, 3636, 2632, 1850, 1500, 1488, 1422, 1189, 1044, 922, 868, 845, 822, 788, 776, 766, 742, 733, 721, 676, 654, 625, 620, 612, 610, 608, 595, 515, 487, 461, 435, 423, 380, 322, 283, 232, 144, 136, 20; Enuresis (bed wetting. Use with parasite sets)—10000, 880, 787, 727; Epicoccum—734, 778; Epicondylitis (tennis elbow, lateral or vertical)—1.2, 250, 728, 766, 776, 880: Epidermophyton_floccinum (fungus that attacks skin and nails, including, some athlete's foot and jock itch ringworms. Also use Microsporum freqs and Fungal general if necessary.)—22107, 465, 784, 644, 766, 345; Epididymitis (inflammation of testicle area, ducts. See also Orchitis.)—2250, 1500, 880, 787, 727, 20; Epilepsy (also try Shigella)—10000, 880, 802, 787, 727, 700, 650, 600, 210, 633, 125.20; Epstein_Barr_-HC—18919.09, 18670.15, 941.93, 929.53; Epstein_Barr_virus (the herpes virus causing mononucleosis)—8768, 6618, 1920, 1013, 825, 738, 669, 667, 663, 660, 274, 253, 1172, 105; Epstein_Barr:virus_secondary (also try General parasite and roundworm freqs)—744, 776, 778, 465. 880, 787, 727, 1032, 1920; Ergot_HC—14687.19, 731.23; Erwinida_amylovoran_HC—17425.47, 867.56; Erwinia_carotovora_HC—18570.58, 924.57; Erysipelas (bacterial infection manifesting in skin inflammation caused by strep pyrogens or other pathogens and possibly related to the swine form of the disease)—616, 776, 735, 845, 660, 10000, 880, 787, 727, 465, 20; Erythema_infectiosum (human parvovirus B19, sometimes known as Fifth disease, a contagious viral infection which causes blotchy or raised red rash with mild illness. exp)—809, 1618, 3236; Erythema_nosodum—9.39; Escherichia_coli (use E. coli); Esophagus (constriction. Also see General antiseptic and dental freqs)—880, 787, 727; Euglena—432, 3215, 3225, 3325, 6448; Eustachian_tube _inflammation (also use General_Antiseptic)—1550, 880, 802, 787, 776, 727, 465; Eye_disorders (blurred vision, cataracts, crossed eyes, diplopia, infections, etc.)—10000, 2010, 1831, 1830, 1829, 1600, 1552, 1335, 880, 787, 727, 496, 400, 360, 350, 160, 20; Eye_inflammation (also see General antiseptic)—1.2, 80; Eyelid_droop (use Ptosis); Eyes_glaucoma—1600, 1830, 880, 787, 727; Eyesight_sharpen—350, 360, 1830;

Facial—10000, 6000, 304, 1131, 33; Facial_paralysis—10000, 880, 787, 727; Facial_toning—1,2; Fainting—20; Fascia (fibrous tissue under the skin. Also see Fibroid fiats)—20; Fatigue_general—428, 424, 664, 660, 464, 125, 120, 95, 72, 20, 444, 1865, 10000, 5000; Febris_wolhynia (a Rickettsia illness, transmitted by lice, that is debilitating and conducive to relapse)—547, 356; Fel_tauri (homeopathic preparation of ox bile)—672; Feli—435, 742; Felis—430, 834, 2232, 3233; Felon_1 (pus, infection of finger tips)—657, 659, 738, 751; Felon_2—663, 665, 720, 722; Febris_Wolyhnica—547; Fever (some causes)—1552, 880, 800, 832, 422, 2112, 787, 727, 20; Fever_sunstroke—20, 440, 880; Fibroadenoma_mainae (non cancerous, fibrous nodules in the breasts. Use kelp capsules too)—1384, 2128. 2189; Fibroids_general (sec Parasites, flukes, general)—2950, 2189, 2128, 2127, 2008, 1744, 1552, 1550, 1488, 1384, 1234, 880, 802, 787, 776, 727, 690, 666, 465, 267; Fibroma—2127, 2008, 727, 690, 666, 1550, 802, 465; Fibromyalgia (run Detox afterwards if needed)—328, 880, 800, 728, 5000, 2720, 2180, 2128, 664, 464, 304, 120, 20; Fibromyalgia_1—120, 140, 304, 464, 728, 800, 880, 2489, 3176, 5000, 6000, 9000; Fibromyalgia_2—2008, 2050, 2080, 2127, 880, 800, 787, 728, 600, 420, 320, 120, 3790, 3792, 3794, 3796, 3798, 3800, 3802, 3804, 3806, 3808, 3810; Fibromyalgia_TR (includes muscle relaxtion frequencies. Run 321, 384, and 40 for 12 min each)—321, 384, 40, 965, 200, 120, 240, 760, 68, 6000, 304; Fibropendulum—661, 7465, 211, 233, 766; Fibrosarcoma—1744; Fibrosis_lung (use Pulmonary fibrosis); Fifth _disease (use Erythema infectiostun); Fischpyrogen—832; Fissures—787, 20, 10000; Fistula_dentalis (727*, 878*, 1122*)—550, 727, 844, 878. 1122.786; Fistula_ulcer (use also Staph freqs)—880, 832, 787, 727; FIV—262, 323, 372, 404, 567, 712, 742, 760, 773. 916, 1103, 1132, 3701; Flatulence—1550, 880, 832, 802, 787, 727, 465; Flu (use Influenza); Flukes (use Parasites, flukes); Fluor_Alb (homeopathic cell salt)—110, 342. 420, 423, 688, 757; Follicular_mange (contagious dermatitis found in many animals that is caused by mites and in which the principle activity is at the hair follicles. Also soak with vegetable oil, let sit at least 15 minutes, and rinse with thyme tea.)—253, 693, 701, 774; Folliculitis_hot_tub (tiny pimples)—174, 482, 5311; Food_poisoning (some classes. Also use Salmonella typhimurium and see General antiseptic, Abdominal pain, and. Abdominal inflammation.)—11552, 802, 832 Foot_and_mouth_syndrome (a mild viral infection found in young children but may also be a factor in ALS)—232, 237, 1214, 1243, 1244, 1271, 5411; Fractures_healing—220, 230, 10000, 880, 787, 727; Frequency_fatigue (tiredness that is not detox from using frequency tone generator or system of frequency tone generators too long)—10.55, 7.8:3; Frigidity_female—10000, 20; Frostbite—880, 787, 727; Frozen_shoulder use strep pneumonia if necessary)—10000, 880, 802, 787, 766, 727; Fruitfly—2167; Fungus_and_mold_v—4442, 24111, 1833, 1823, 1333, 1155, 1130, 1016, 942, 933, 886, 880, 866, 784, 774, 766, 745, 743, 728, 623, 623, 594, 592, 565, 555, 524, 512, 464, 414, 374, 344, 337, 321, 254, 242, 222, 158, 132; Fungus_EW_range—823, 824, 825, 826, 827, 828, 829; Fungus_flora_1—331, 336, 555, 587, 632, 688, 757, 882, 884, 887; Fungus_foot_and_general_1(use 1550 for 30 min)—1550; Fungus_general (also see candida, yeast, and other specific types)—2222, 1552, 1550, 1153, 1134, 1016, 880, 802, 787, 784, 727, 582, 465, 422, 254, 72, 20; Furunkulosis (boils on skin. Also use Staph amens freqs)—1550, 1000, 880, 802, 787, 784, 770, 728, 727, 660, 465, 200, 116, 20; Furunkulosis_herpes (also see Herpes general and Furunkulosis)—200, 1000, 1550, 802, 787, 727; Furunkulosis_TR (also use Staph aureus)—1550, 1000, 880, 802, 787, 784, 770, 728, 727, 660, 465, 200, 116, 20; Fusarium_general—768, 625, 746; Fusarium_oxysporum (a fungus causing inflammation of the cornea of the eye)—102, 332, 705, 795, 780;

Gaffkya_tetragena_HC (causes respiratory infections)—17425,47, 867,56; Gardnercila_vaginalis_HC (causes ovarian and genital tract infection)—16927.60, 842.78; General_antiseptic—10000, 5000, 3176, 21145, 1552, 1488, 880, 802, 786, 776, 766, 760, 728, 688, 683, 676, 666, 660, 464, 450, 444, 428, 120, 20; General_antiseptic_1—10000, 5000, 3176, 2720, 2000, 1865, 1770, 1550, 1360, 880, 804, 802, 787, 786, 760, 728 690, 660, 590, 555, 465, 450, 428, 768, 523, 333, 14; General_demo (greatest hits)—10000, 4412, 3040, 2128, 2112, 1862, 1550, 880, 800, 786, 732, 728, 712, 688, 676, 644, 464, 422, 128, 120, 20; General_malady—40000, 10000, 5000; General_prog_Blaster5—2489, 2128, 2008, 2000, 1998, 1840, 1800, 1600, 1570, 1560, 1550, 1500, 880, 800, 787, 784, 776, 740, 727, 690, 666, 622, 600, 500, 400, 120, 80; General_program_EMEM—720, 1550, 20, 4200; General_prophylaxis—20, 64, 95, 125, 225, 427, 440, 664, 728, 784, 880, 802, 832, 680, 760, 1550, 464, 10000, 676, 1488; Geotrichum_candidum (fungus found in feces and dairy products whose manifestations resemble those of candida)—350, 355, 384, 386, 403, 404, 407, 409, 410, 412, 415, 418, 543, 544, 687, 987, 988, 737, 700; German measles (Use Measles, rubella); Giardia (use Parasites, giardia); Gingivitis (inflammation of gums. See also Dental, Dental foci, Stomathis. Take calcium and magnesium internally and brush with CoQ10 and myrrh or goldenseal)—1550, 880, 802, 787, 728, 726, 465, 20, 1556, 776, 465; Glanders (use Pseudomonas mallei); Glandular fever—10000, 20; Glandular_fever_thyroid (exp)—10000, 20, 16000; Gliociadium (brain fungus)—855, 469, 633; Goiter (use Struma); Gonads inflammation (use Orchitis); Gonorrhea—7120, 6000, 2330, 1500, 712, 660, 600, 233, 150; Gonorrhea_neisseria_HC—16628.88, 927.90; Gout (Also use water cure and high dose bromelain or other enzymes. See also Kidney freqs)—9.39, 3000, 10000, 880, 787, 727, 20; Granuloma_dent—441; Gravel_urine—2.65, 3000, 880, 787, 727, 20; Graves_disease_and_goiter—20, 727, 787, 880; Greatest_Hits (use General demo); Green_Dye—563, 2333; Grippe (use Influenza, grippe); Griseofulvin—14338.68, 713.88; Gulf_War_Syndrome_v—7755, 6007, 5419, 2688, 1370, 1365, 975, 866, 837, 771, 714, 639, 633, 578, 533, 435, 392, 255, 253, 136; Guillain_Barr_Syndrome_TR—10000, 7344, 5000, 2950, 2900, 2650, 2600, 1550, 1234, 740 880, 787, 727, 330, 165, 82.6, 41.75, 20.87, 30, 7344, 4412, 5000; Gum_disease (use Gingivitis_Pyorrhea); Haemophilia_tonic (hereditary bleeding disorders in which the blood does not readily clot)—778, 845, 751;

Haemophilus_influenzae_HC (causes bacterial meningitis, infects joints)—16728.45, 832.86; Hair_human—646; Halitosis (bad breath. See also Strep pneumonia, Strep aureus, Pharyngitis, Dental, Parasite general, and General antiseptic sets)—1550, 802, 880, 787, 727, 20 Hallucinations—10000, 880, 787, 727, 20; Hand_foot_and_mouth_syndrome (a mild viral infection found in young children)—232, 237, 1214, 1243, 1244, 1271, 5411; Hangover (see also Kidney and Liver support freqs)—10000, 522, 146; Hayfever (only some types)—880 787, 727, 20, Head_injury_followup (Seek immediate medical attention for head injuries!)—9.6, 3000, 880, 787, 727, 522, 72, 5.8, 4.9; Headaches—144, 160, 1.2, 520, 10, 10000, 304; Headaches_caused_by_vertebral_misalignment (Not a substitute for adjustment)—9.6, 3000, Headaches_comp—10000, 3000, 880, 787, 727, 650, 625, 600, 522, 520, 304, 160, 146, 144, 125, 95, 73, 20, 10, 9.6, 9.39, 7.83, 6.3, 5.8, 4.9, 4, 1.2, Headaches_due_to_parasites (see also Parasites, strongyloides)—125, 95, 73, 20 727, 3000; Headaches_due_to_toxicity—522, 146, 4.9, 3000, 880, 787, 727, 20; Headaches_unknown_cause—10, 4, 5.8, 6.3 7.83, 3000, 650, 625, 600 Headaches_urogenitally_caused—9.39, 3000; Healing_and_regeneration—2720, 266, 47; Heart_blockage_1—59, 60, 61; Heart_general—20, 81, 162, 5000; Heart_tonic (lab animals only. See also Chlamydia Staph infection, Circulatory stasis, and Kidney freq sets)—80, 160, 20, 73, 3,9, 3000, 880, 787, 727, 465, 162, 125, 95, 20, 1.2; Heartburn_chronic (use calcium magnesium supplement and The Water Cure. Also see Staph and Strep general sets and H. pylori)—676, 832, 2720, 2170, 2127, 1800, 1600, 1550, 802, 1500, 880, 787, 727, 685, 465, 444, 1865, 125, 95, 72, 20; Heartworm (use Parasites, heartworm); Heavy_metal_toxicity_TR—31905.6, 38014.4, 39032.6.43644.3, 56656.4, 76677.5, 43644.3, 38014.3, 4202.3, 5333.7, 9887, 1902, 317; Heel_spurs (use Bone_spurs); Helicobacter_pylori (ulcer. Run 676 for 10 min, 676*)—2950, 2819, 2779, 2167, 880, 728, 705, 695, 676, 352, 347; Helicobacter_pylori_1—2167, 724, 880, 2950; Helicobacter_pylori_2—695, 2779, 705, 2819; Helicobacter_pylori_3—3—0.2, 0.4, 0.6, 0.8; Helminthosporium (the reproductive element of some parasitic worms)—793, 969; Hemobartinella_felis—603, 957; Hemophilia—603; Hemorrage—1550, 802; Hemorrhoids—4474, 6117, 774, 1550, 447, 880, 802, 727; Hepatitis_A (add Hepatitis general freqs if necessary)—321, 346, 414, 423, 487, 558, 578, 693, 786, 878, 3220, 717; Hepatitis_B (add Hepatitis general freqs if necessary)—334, 433, 767, 869, 876, 477, 574, 752, 779; Hepatitis_B_HC (antigen)—20562.06, 1023.72; Hepatitis_C (also try Parasites, schistosoma mansoni and Hepatitis general freqs if necessary)—5000, 3220, 3176, 2489, 2189, 1865, 1600, 1550, 1500, 1371, 933, 931, 929, 880, 802, 665, 650, 633, 625, 528, 444, 329, 317, 250, 224, 166, 146, 125. 95, 72, 28, 20; Hepatitis_C_1—10000, 5000, 3220, 3176, 2489, 1865, 1600, 1550, 1500, 880, 802, 665, 650, 600, 444, 250, 166. 146, 125, 95, 72, 28, 20; Hepatitis_C_TR—728, 166, 224, 317, 727, 787, 880, 2189; Hepatitis_general—1550, 1351, 922, 880, 802, 727, 477, 329, 317, 224, 28; Hepatitis_general_secondary—284, 458, 477, 534, 788, 922, 9670, 768, 777, 1041; Hepatitis_general_v—987, 934, 922, 878, 876, 842, 786, 781, 563, 562, 558, 534, 528, 477, 334, 321, 317, 224, 213, 166; Hernia—10000, 787, 727, 2720, 5000; Herpes_general (use 1488 and 2950 for 15 min, 1488*)—2950, 1900, 1577, 1550, 1489, 1488, 629, 464, 450, 383, 304, 165, 141; Herpes_general_secondary—37000; Herpes_general_v—165. 141, 383, 450, 629; Herpes_progenetalis (genital. Also use Herpes simplex II)—141, 878, 898, 5310, 440, 171, 660, 590, 1175; Herpes_siinplex_I (secondary. Cold sores: primarily non-genital, first try Herpes,gerieral)—322, 476, 589, 664, 785, 822, 895, 944, 1043, 1614, 2062, 1489, 2950; Herpes_simplex_I_1—339, 343, 480, 591, 778, 782, 843, 1614, 657, 699, 700, 734; Herpes_simplex_I_2—2489, 1800, 465, 1550, 1500, 880, 787, 727, 1850, 428, Herpes_simplex_I_3—470, 647, 648, 650 652, 654, 656, 658, 660, 847, 5641, 8650; Herpes_simplex_1_4 (use 2950 for 20 min)—2950; Herpes_simplex_I_HC—14537.82, 17201.43, 723.80, 856.41; Herpes_simplex_I_1—556, 832, Herpes_simplex_II_HC—17923.34, 17674.41, 892.35, 879.96; Herpes_simplex_IU2—808, Herpes_simplex_RTI (based on Resonant Light Technology's herpes set, most frequencies contracting spread. Also can be used for measles, chicken and small pox, mononucleosis, shingles, rubella, cold sores, epstein ban, variola, stoma stoinatosis, and pyorrhea.)—10000, 5000, 3176, 2489, 186, 372, 427, 446, 465, 484, 503, 522, 541,560, 579, 598, 617, 636, 655, 674, 693, 712, 731, 750, 769, 788, 807, 826, 845, 864, 883, 902, 921, 940, 959, 978, 997, 1016, 1035, 1054, 1073, 1488, 1550, 1568, 1644, 1865, 1909, 2976, 5310, 5952, Herpes_TR (run set three times, pausing 10 minutes between runs. All frequencies can run 60 sec except 2950, which runs 180 sec)—2950, 322, 476, 468, 589, 664, 785, 822, 895, 936, 944, 1043, 1614, 1871, 2062, 1489, 3742, 748, Herpes_type_1_anec_comp (combines most anecdotal freqs for Simplex I)—8650, 7484, 5641, 3742, 2950, 2489, 1871, 1850, 1800, 1614, 1550, 1500, 935.5, 880, 847, 843, 787, 782, 778, 734, 727, 700, 660, 656. 652, 648, 591, 480, 467.8, 428, 343, 339; Herpes_type_2_comp—8778, 1402, 888, 880, 848, 846, 832, 808, 776, 732, 717, 685, 665, 556, 540, 532, 528, 373, 370, 366, 362; Herpes_type_2A—532, 848; Herpes_type_2A_secondary—360, 362, 364, 366, 368, 370, 373, 528, 685, 846, 880, 888, 8778, 540, 665, 716, 717, 718, 731, 732, 733, 776, 1402; Herpes_type_5 (cytomegalovirus. 2145*)—126, 597, 629, 682, 1045, 2145, 8848, 8856; Herpes_type_C—395, 424, 460, 533, 554, 701, 745, 2450; Herpes_zoster (chickenpox, shingles. 664*)—3343, 2320, 2170, 1600, 1500, 1160, 914, 880, 833, 802, 787, 664, 580, 3.9; Herpes_zoster_1 (4 min each freq)—10000, 833, 802, 3.9; Herpes_zoster_secondary—1550, 802, 1800, 1865, 728, 2720, 2128, 5000, 464, 800, 574, 1557, 304, 20; Herpes_zoster_v—7160, 3343, 2431, 2323, 1577, 1544, 40887, 958, 934, 787, 786, 738, 718, 716, 686, 668, 643, 576, 574, 573, 572, 563, 554, 542, 453, 446, 436, 425, 423, 411, 345, 333, 223, 134; Hiatal_hernia (use staph and strep sets); Hiccups—20, 10000; Hip_pain (use also Arthritis)—880, 787, 727, 20; Hirudo_medicinalis (a homeopathic remedy prepared from a leech used for therapeutic purposes)—128; Histomonas_meleagridis_HC—18769.72, 934.49; Histoplasma—424, 616, 749; Histoplasma_capsulatum_HC—15035.69, 748.58; HIV (see also AIDS)—683, 714, 3554, 830, 450; HIV_HC—18172.28, 904.74; Hives (use Urticaria); Hoarseness—880, 760, 727; Hodgkins_disease (use Cancer, Hodgkin's disease); Hookworm (use Parasites hookworm); Hordeolum (use Stye); Hormodendrum (a genus of fungi that includes human pathogens)—663, 678, 695, 532, 627; Hormonal_imbalances—5.5; Hot_flashes (complications)—10000, 880, 787, 727; Hot_tub_folliculitis (use Pseudomonas freqs); Household_Insect_Mix—723, 100; Human_T_lymphocyte_Virus1 (used for MS and autoimmune disease)—725, 2432, 243, 6353, 732, 844, 646; Human_T_lymphocyte_Virus2 (used for Gulf War syndrome)—725, 1230, 245, 314, 965; Human_T_lymphocyte_Virus3 (used for AIDS)—633, 1220, 6230, 8225, 111, 392, 776, 837, 1675, 2664, 3806, 714; Human_T_lymphocyte_Virus4—444, 2323; Human T_lymphocyte_Virus5—83, 235, 645, 2323, 3432, 4093, 5532; Human_T_lymphocyte_Virus6—702, 747, 2245, 183; Hydrocele (fluid in testicle, etc.)—880, 787, 727; Hyperacidity_stomach—7.82, 20, 230; Hyperosmia (overacute smell and taste)—20, 10000, 522, 146; Hyperparathyroidism—4.6, 9.5, 9.6; Hypertension—3176, 2112, 20, 95, 324, 528, 15, 9.19, 7.83, 6, 10000, 880, 787, 727, 304; Hypertension_reni_induced (red high, diastolic high pressure. Also see Kidney freqs)—9.19, 6; Hypertension_spastic—95; Hyperthyroid—3, 0.5; Hypochondrium_upper_abdomen—20, 10000; Hypophyseal_disturbances (pituitary)—4; Hypotension (low blood pressure)—20, 727, 787, 880; Hypothyroid—2, 12, 35. 16000, 10000, 160, 80, 20; Hypoxia (low oxygen, use also Circulatory stasis)—727, 787, 880, 10000;

Icterus_haemolytic (a chronic form of jaundice involving anemia)—243, 768; Ileocolitis_colon_inflammation (also use Colitis if necssary and see Parasite general and roundworm sets)—802, 832, 440; Immune_system_stabilization_TR—330, 10000, 7344, 5000, 1550, 1234, 740, 880, 835, 787, 727, 30; Immune_system_stimulation—10000, 5611, 5000, 4014, 3448, 3347, 3176, 2929, 2867, 2855, 2791, 2720, 2489, 2180, 2128, 2008, 1862, 1488, 880, 800, 787, 728, 665, 464, 432, 304, 120, 20, 8; Immune_system_stimulation_1—40.4, 40.5, 40.6, 40.7, 40.8; impotence (many classes. Also use Circulatory stasis)—9.39, 2127, 2008, 465, 10000, 880, 802, 787, 727, 690, 666, 125, 95, 73, 72.20, 650, 625, 600; Incontinence_1 (use all freqs for 10 min)—2250, 2050, 2128, 690, 666; indigestion (use Dyspepsia); Infantile_paralysis (also use Polio)—1500, 880, 787, 727, 776, 5000, 10000; Infections_general (see General Antiseptic)—1550, 880, 802, 786, 728, 465, 444, 125, 95, 72, 48, 20, 304, 1.2, 5500, 676, 422, 766; Infections_general_secondary—5000, 1600, 1500, 832, 776, 760, 700, 690, 666, 650, 625, 600, 1865, 500, 450, 440, 428, 660, 2112; Infections_general_tertiary—1800, 2720, 2489, 832, 3040, 2008, 1850, 610, 732, 751, 40000; Infertility (see also Impotence)—2127, 2008, 465, 880, 802, 787, 727, 690, 666, 650, 625, 600, 9.39; Inflammnation_general (also see infection general sets)—1.5, 3.6, 2720; Influencinum_Berlin55—430, 720, 733, 787; Influencinum_vesic_NW—332, 364, 519, 588, 590, 238, 239, 715; Influencinum_vesic_SW—433, 645, 658, 824; Influencinum_nvesica_general (blisters)—203, 292, 588, 612, 975, 407.682; influenza (mutates to new strains constantly but these may be helpful. See also Influenza. Grippe and influenza Virus. Use all freqs for 5 min if necessary)—7766, 7764, 7762, 7760, 7344, 5000, 3672, 2720, 2050, 2008, 1947, 1674, 1550, 1500, 1234, 885, 880, 875, 800, 786, 728, 683, 512, 464. 440, 304, 20; Influenza_1_99_00—10000, 880, 727, 330, 728; Influenza_1957_A_Asian—768, 574; Influenza_1978—844, 814, 610; Influenza_1979—123, 513, 522, 565, 788, 601; Influenza_1983—424, 730, 734, 428; Influenza_1989—216, 322, 627, 703, 748; Influenza1993—522, 615, 778, 850, 959; Influenza_11993_secondary—207, 254, 580, 582, 584, 586, 588, 848, 947, 7967, 8910, 739, 741, 760, 765, 773, 915; Influenza_1994—689, 697, 699, 798; Influenza_1994_secondary—337, 690, 868, 869, 702, 727, 729, 776, 779; Influenza_1997_1998—2008, 880, 787, 727, 35, 20, 465; Influenza_2_9900—440, 465, 613, 666, 727, 787, 800, 1000, 5000, 10000; Influenza_2003_2004_1—7344, 3672, 1234, 880, 743, 727, 683, 464, 452, 333, 72, 200, 93.9, 2720, 2489, 1800, 1600, 1550, 885, 875, 832, 802 787, 776, 766, 1500, 832, 787, 650, 625, 600, 465, 144, 582; Influenza_2003_2004_2—8911.25, 9113,5, 9141,5, 9393.13, 6654.69, 7255, 7284.06, 8041.5, 10334.06, 10346,56, 10976.38, 7414.28, 7631.09, 7632.66 7667.38, 7676.94, 8045.81, 8082.59, 8305,19, 5548.59, 5549,22, 5554.69, 5554,84, 5558.75, 5560.78, 5562.5, 7118.2, 5045.03, 6752.01; Influenza_2005-2006_TR—2950, 322, 476, 468, 589, 664, 785, 822, 895, 936, 944, 1043, 1614, 1871, 2062, 1489, 3742, 748; Influenza_2005-2006_TR—2748.8, 5695.9, 2856.7, 2935.4, 4159.2, 4534.4, 2276,3, 2504,5, 3229.4, 6466.6, 1715,1, 4633.9, 4769.4, 1192.6, 599, 2390, 1257.2, 315.7, 5190,7, 1734, 6936.5, 867.9, 434, 217.1, 6951, 4448.9, 6306,3, 4220, 8478, 1216, 1214, 1060, 1010, 935, 778, 777, 465, 457, 445, 433, 425; Influenza_A_and_B_HC (flu shot)—15931.86, 15682.93, 793.20, 780.81; Influenza_aches_and_respiratory (use 7760 and 7766 for 10 min, all others for 5)—440, 512, 683, 728, 784, 787, 800, 875, 880, 885, 2050, 2720, 5000, 7760, 7766, 304; Influenza_and_Cold_1 (winter 01 to 02)—959, 962; Influenza_Asian_grippe_A—516, 656, 434; Influenza_aviain_exp_TR (sweep from 4896.1 to 5060.9 by 0.03125 dwell 1); influenza_autumn1998 (use 8700 and 7760 for 15 min)—250, 465, 8210, 8700, 7760; Influenza_Bach_Poly—122, 350, 487, 572, 634, 768, 823, 1043, 1272, 764, 771; Influenza_general_1 (winter 98 to 99)—6984, 6986, 6988, 6990, 6992, 6994, 6996, 6998, 7000, 7002, 7004, 7006, 7008, 7010, 7012, 7014, 7016; Influenza_grippe_1986_tri—532, 588, 660, 662, 664, 666, 668, 670, 994, 462, 712; Influenza_grippe_1987—140. 332, 581, 953, 4868, 730; Influenza_grippe_1988—267, 536, 568, 2050, 752, 781; Influenza_grippe_1989—353, 536, 874; Influenza_grippe_1990—541, 560, 656; Influenza_grippe_general—343, 500, 512, 541, 862, 1000, 1192, 3012, 3423, 10223; Influenza_grippe_vapch—153, 343; Influenza_haemophilus—542, 552, 885, 959, 734, 633.1, 2532.4; Influenza_haemophilus_type_B (can cause a type of meningitis)—652, 942, 483, 731, 746; Influenza_spanish—462, 787; Influenza_2005_sweep_TR (long sweep from 9750 to 10156.25 by 0.03125 steps at 0.25 sec duration/step); Influenza_overnight_TR (Combines many of the CAFL flu frequencies into on large set. TR specifies to pulse 0.25 90, use duration of 90 sec per freq to make approx 8 hr set)—10223, 10000, 9090, 8910, 8700, 8250, 8210, 8000, 7967, 7766, 7764, 7762, 7760, 7344, 6984, 6986, 6988, 6990, 6992, 6994, 6996, 6998, 7000, 7002, 7004, 7006, 7008, 7010, 7012, 7014, 7016, 5608, 5000, 4868, 3672, 3423, 3122, 3012, 2720, 2544, 2532.4, 2489, 2558, 2398, 2050, 2008, 1947, 1922, 1800, 1674, 1600, 1550, 1500, 1422, 1272, 1242, 1234, 1215, 1192, 1191, 1186, 1043, 1020, 1000, 995, 994, 985, 984, 969, 967, 962, 959, 954, 953, 947, 946, 945, 942, 932, 922, 915, 895, 889, 885, 880, 878, 875, 874, 869, 868, 863, 862, 861, 850, 848, 844, 841, 839, 836, 833, 832, 823, 814, 802, 800, 798, 788, 787, 786, 784, 782, 781, 780, 779, 778, 777, 776, 773, 771, 770, 768, 766, 765, 764, 763, 762, 761, 760, 758, 757, 756, 753, 752, 750, 748, 746, 745, 743, 742, 741, 740, 739, 734, 732, 731, 730, 729, 728, 727, 726, 724, 722, 720, 715, 713, 712, 703, 702, 699, 697, 690, 689, 687, 683, 680, 679, 678, 676, 674, 672, 670, 668, 666, 664, 663, 662, 660, 656, 653, 652, 651, 650, 649, 647, 640, 632, 634, 633.1, 627, 625, 615, 613, 610, 601, 600, 597, 588, 582, 581, 580, 574, 572, 568, 566, 565, 560, 558, 555, 553, 552, 542, 541, 537, 536, 535, 534, 532, 530, 525, 524, 523, 522, 518, 516, 513, 512, 500, 496, 487, 483, 482, 472, 469, 468, 465, 464, 462, 461, 457, 452, 447, 442, 440, 434, 432, 428, 425, 424, 421, 413, 387, 360, 358, 356, 354, 353, 352, 350, 345, 343, 341, 340, 338, 337, 336, 334, 333, 332, 330, 324, 322, 316, 304, 272, 267, 254, 250, 232, 216, 207, 200, 153, 144, 140, 123, 122, 93.9, 72, 47, 35, 20; Influenza_triple_nosode—421, 632, 1242, 1422, 1922, 3122; Influenza_V_grippe—861; Influenza_V2_grippe—324, 652, 653; Influenza_V3 grippe—550, 553; Influenza_V4_grippe—232, 352, 2558; Influenza_V5_grippe—945, 518; Influenza_V75_Victoria—343, 316, 1020; Influenza_VA2_grippe—3:34, =172, 496, 833, 836, 922, 728; Influenza_VA2L_grippe—447; Influenza_vitus_1991_1992—153, 345, 387, 758, 984, 985; Influenza_virus_1991_1992_secondary—330, 332, 334, 336, 338, 340, 350, 352, 354, 356, 358, 360, 525, 632, 740, 761, 762, 776, 777, 780; Influenza_virus_1992_1993—535, 946; Influenza_virus_1992_1993_secondary—272, 534, 566, 668, 674, 776, 782, 947 632, 640, 713, 715, 742, 773, 777; Influenza_virus_1993_1994—757, 885, 895, 969; Influenza_virus_1993_1994 secondary—447, 457, 597, 756, 764, 776, 798, 878, 967, 9090, 663, 720, 728, 729, 745, 762, 764, 770, 773, 779; Influenza_virus_A—322, 332, 776; Influenza_virus_A1974—442; Influenza_virus_A Port_Chalmers—622, 863; Influenza_virus_B—468, 530, 532, 536, 537, 568, 722, 740, 742, 744, 746, 748, 750, 1186, 679; Influenza_virus_B Hong_Kong—555; Influenza_virus_British—558, 932; Influenza_virus_general—728, 800, 880, 7760, 8000, 8250; Influenza_virus_swine—413, 432, 663, 839, 995; Influenza_with_Fever_v—954, 889, 841, 787, 763, 753, 742, 523, 513, 482, 469, 461, 425, 341, 332; Influenza_with_respiratory_1 (winter 99 to 00)—47, 1191, 2398, 2544, 5608, 7760, 7766, 672, 674, 676, 678, 680, 647, 649, 651, 653, 1215, 724, 726, 728, 730, 732, 746, 768, 687; Influenzum_toxicum—854; Injection_allergic_reaction—10000; insomnia (see also Parasite general set)—3.59, 3, 7.83, 10, 1550, 1500, 880, 802, 6000, 304; Intelligence_and_clarity_of_thought—20, 10000; Intercostal_neuralgia (pain in rib musculature)—3000, 1550, 880, 802, 787, 776, 727, 125, 20, 1865, 444; Interleukin (used to stimulate lymphocyte production)—3448, 2929, 4014, 5611, 2867, 2855, 2791; Intermittent_claudication—45, 48; Intestinal_problems_colon (see also Colitis)—10, 440, 880, 787, 727; Intestinal_problems_general (also see Parasite general set and General antiseptic)—802; Intestines_inflammation (see also Colitis)—727, 787, 880, 832, 1550, 105, 791; Iodamoeba_butschii_HC—22155.25, 1103.04; Irritable_bowel_syndrome (see also Parasites general set and Colitis)—6766, 5429, 4334, 2018, 1550, 880, 832, 829, 812, 802, 787, 727, 465, 422, 407, 334, 20; itching (pruritis. Take hot bath with a cup of apple cider vinegar afterwards. If chronic and no long term relief, use Parasite sets including Parasites general and Parasites blood flukes.)—880, 787, 727, 444, 125, 95, 72, 20, 1865, 3176;

Jaundice (see also Liver support, gall bladder, Leptospirosis, and Parasites fluke and general frequency sets)—5000, 1600, 1550, 1500, 880, 802, 650, 625, 600, 444, 1865, 146 250, 125, 95, 72, 20; Jock_itch (use Epidermophyton floccinum); Joint_inflammation (also see Arthritis sets)—10000; Joint_pain_basic_TR (also known as Pain.Bio, run 160, 500, 1600, 5000 with pulse 4 60, 1600 500 160 with pulse 2 60, 160 324 528 with pulse 2 60 converge 1 1)—160, 500, 1600, 5000, 324, 528;

Kaposis_sarcoma (use Cancer_Kaposis_sarcoma); Kidney_insufficiency—9.2, 10, 40, 440, 1600, 1550, 1500, 880, 802, 650, 625, 600, 444, 1865, 146, 250, 125, 95, 72, 20; Kidney_papilloma (small, usually benign growth on a kidney)—110, 148, 264, 634, 767, 848, 917, 760, 762, 1102; Kidney_stimulation_TR (specified by True Rife as may help against infections common in acute renal failure)—4412, 2720, 2400, 2112, 1864, 1600, 1550, 1500, 880, 854, 800, 784, 751, 732, 728, 712, 668, 651, 644, 625, 600, 524, 465, 442, 334, 250, 240, 152, 146, 28, 125, 120, 112, 100, 96. 93, 72, 64, 40, 20, 2222, 1153, 1134, 1016, 582, 676, 440; Kidney_stones (use with vitamin, mineral, and herb supplementation)—444, 727, 787, 880, 10000, 6000, 3000, 3.5, 1552; Kidney_ionic_general—440, 248.8, 880, 20, 10000, 800, 5000, 3000; Kieferosteitis (a type of bone inflammation marked by enlargement and pain)—432, 516, 384; Klebsiella_pneumoidae (use Pneumoniae klebsiella); Knee_joint_pain—1550, 880, 802, 787, 727. 28, 20, 7.69, 3, 1.2, 250, 9.6, 9.39;

L_lysine (stimulates)—195.5, 391, 782, 1564.1, 3128.2, 6256.4; Lac_deflorat—230, 371, 232, 2121; Lactobacillus_acidophilus_HC—17375.69, 865.08; Lamblia (use Parasites, Giardia); Large_intestine_tonic—8, 440, 880; Laryngeal_polyp—765, 202; Larynx—10, 440, 465, 444, 1550, 880, 802, 787, 727, 28, 7.69. 3, 1.2, 250, 9.6, 9.39; Lateral_sclerose (degeneration of spinal cord leading to spastic paraplegia. See also ALS.)—254; Laxative_mild—802; Legionella (Legionnaires' disease, caused by a gram-negative bacteria associated with condensed or treated water that migrate to lung tissue and stimulate severe respiratory manifestations, fever, headache, abdominal pain, and may affect kidneys and liver.)—723, 724, 897, 975, 8120, 8856, 690, 693; Leiomyoma (use Fibroma); Leishman_Donovan (use Parasites, Leishmania); Leprosy—10000, 6000, 743, 600, 252; Leprosy_secondary _infection—1600, 1550, 1500, 880 832, 802, 787, 776, 760, 727, 700, 690, 666, 650, 625, 600, 465, 444, 20, 500, 450, 440, 428, 660; Leptospira_interrogans_HC—19865.04, 989.02; Leptospirosis (a spirochete bacterial disease that is spread to humans through animal urine that can cause meningitis, jaundice, anemia, miscarriage, and death)—612, 663; Leukocytogenesis_stimulation (sec also Immune system stimulation)—20, 727; Leucocytozoon_HC—19914,83, 991,50; Leukoderma_acquired (use Vitiligo); Leukoencephalitis (inflammation of brain's white matter, usually in infants and children, but also found in horses as a result of forage poisoning)—324, 572, 934, 1079, 1111, 1333; Leukoencephalitis_secondary—338, 783, 932, 1035, 1160, 1630, 712, 713, 715, 1244; Leukoplakia (white patches on mucous membranes, also see EBV, Papilloma, and Cancer, carcinoma freqs)—465, 2127, 2008, 727, 690, 666; Leukose (proliferation of tissues that form white blood cells; considered to be foundational stage of leukemia. See also Cancer Leukemia, Parasite general, Parasite fluke sets)—612, 633, 653, 3722, 644; Lichen_planus _TR—728, 166, 224, 317, 727, 787, 880, 2189, 7344, 4412, 1550, 1234, 740, 880, 787, 727, 330, 10000, 5000, 30; Lichen-sclerosus—1600, 500, 160, 324, 528, 7344, 4412, 1550, 1234, 740, 880, 787, 727, 330, 10000, 5000, 30; Lipoma (benign, soft tumor of fatty tissue. Also use Liver support.)—47, 606, 709; Listeriose (a serious disease causing miscarriage, meningitis, and endocarditis in humans; known as circling disease in ruminants and causes liver necrosis in animals with single stomachs. Also see Strep general and General infection)—377, 471, 626, 628, 634, 774, 2162, 7867, 714, 724; Liver_enlargement—727, 787, 880; Liver_fluke (Use Parasites, flukes); Liver_necrosis (use Listeriose, Liver support, Parasites schistosoma mansoni); Liver_stores_bile_cholesterol—21.34; Liver_support (also try E_coli, Parasites_general and Parasites_flukes_general)—33.13, 1552, 802, 751, 331.3; Lockjaw (use Tetanus), Locomotor_dysfunction_incoordination (may be slow results. See also Schumann response)—10000, 880, 787, 776,72 650, 625, 600 444, 1865, 125, 95, 72, 20; Low_blood_pressure (use hypotension), Luesinum_Syphilinum (use Syphillis): Lumbago—10000, 800 880, 787, 727, 125, 95, 72, 444, 1865, 9.19, 8.25, 7.69, 300: Lung (general)—9, 3672; Lung_abscess (use Nocardia asteroides); Lung_infection_1—10.3. 10.5, 11.8, 11.9. 12.1, 12.4; Lung_general_tonic—318, 307.9, 568; Lung_sinus_bacteria—244, 1466, 597, 1311; Lupus_general (localized degeneration of skin by various diseases; vulgaris is a common form of this ailment that is actually a rare form of tuberculosis that manifests with disfigurement and destruction of the skin and cartilage of the face. Also use Parasite fluke sets for any type. Use 633 for 5 min, 2125 and 3612 for 4 min, 2008 and 802 for 8 min)—3612, 2489, 2125, 2010, 2008, 2006, 942, 802, 800, 798, 702, 633, 632, 442, 386, 243; Lupus_general_secondary—205, 244, 352, 633, 847, 921, 993, 1333, 1464, 7865, 771; Lupus_SLE_secondary—2180, 2128, 2008, 1552, 880, 784, 664, 304, 386, 678, 481; Luxation (dislocation of organs or joints)—9.1. 110, Lycogala_HC—627:3.17, 312.32; Lyme_and_Rocky_Mtn_Spotted_Fever_v—7989, 1590, 239, 846, 422, 417, 1455, 39975, 40439, 884, 797, 758, 693, 673, 577, 4870, 4880, 578, 128, 579; Lyme_thsease (also known as borreliosis; relapsing fever in humans and animals caused by parasitic spirochetes from ticks. Also use Babesia if necessary.)—6870, 6863, 46866, 46851, 34170, 34112, 4200, 2050, 2016, 1520, 1455, 920 884, 800, 797, 758, 673, 625, 615, 605, 432, 345, 344, 338, 254; Lyme_1—864, 495, 485, 490, 495, 500, 505, 625, 610, 615, 620, 625, 630, 690 790, 785, 790, 795; Lyme_2 (use 625 for 10 min, 615 for 5 min)—10000, 6870, 6863, 4200, 2720, 2050, 2016, 1520, 1455, 943, 920 885, 884, 880, 864, 800 797, 795, 790, 785, 758, 732, 727, 699, 690, 688, 673, 664, 673, 660, 644, 630, 625, 620, 615, 610, 605, 597, 534, 533, 525, 510, 505, 495, 485, 490, 500, 484, 432, 345, 344, 338, 306, 254, 230, 3; Lyme_3—27735768, 1380882,58, 68750,10, 3422.87; Lyme_4 (use 2016 and 625 for 10 min, others for 5 min)—2050, 1520, 615, 2016, 625; Lyme_5 (use 920 for 10 min)—920; Lyme_6 (borrelia afzellii)—387500; Lyme_7 (borrelia burgdorferi)—380000; Lyme_8 (borrelia garinii)—382000; Lyme_hatchlings_eggs—640, 8554, 203, 412, 414, 589, 667, 840, 1000, 1072, 7087, 1105; Lyme_JB—27735768; Lyme_secondary (254*)—254, 525, 597, 644, 885, 699; Lyme_tertian—306, 432, 484, 610, 625, 690, 864, 2016, 790; Lyme_TR_A (Program A, run every other day)—6675, 4879, 2899, 2720, 2016, 1800, 1600, 1550, 1519, 1455, 1433, 885, 880, 863, 828, 802, 786, 776; Lyme_TR_B (Program B, run every other day)—765, 758, 749, 726, 672, 604, 600, 581, 464, 451, 432, 345, 250, 144, 62; Lymph_plaque—596, 346; Lymph_stasis (use 3176 for 10 minutes)—3176; Lymph_stasis_secondary (also see Lymphangitis)—6.3, 148, 522, 146, 444, 440, 880, 787, 727, 10000, 2.5, 465, 10; Lymph_support—15.05, 10.36, 3176; Lymphangitis (lymphatic vessel inflammation of humans and horses most commonly caused by strep but also by other bacteria, yeast fungus, and cancer. See also Strep General)—880, 574, 778, 1120, 1078, 3176; Lymphogranuloma (use Cancer, Hodgkin's disease); Lymphs_and_detox—10000, 3177, 3176, 3175, 880, 787, 751, 727, 676, 635, 625, 522, 465, 444, 440, 304, 148, 146, 15.2, 15.05, 10.36, 10, 7.83, 6.3, 2.5; Lymph_mover—100000; Lyssinum (use Rabies); Macular_degeneration_and_visual_acuity (use with Cataracts freqs if necessary, 1830*)—8, 1830, 1832, 1834, 1836, 1838, 1840, 1842, 1844, 1846, 1848, 1850, 1852, 1854, 1856, 1858, 1860;

Macular_degeneration_1—0.6, 1.1, 1.4, 1,9, 9,9, 10, 21, 23.6, 24, 25.6, 26.7, 32, 34,1, 410; Magnesium—480; Malabsorption_syndrome (use with Parasites, General set)—727, 787, 880, 800, 1552, 3000; Malaria (an infectious disease, originating in tropical areas, that is transmitted by a mosquito bite and characterized by fever, anemia, and spleen enlargement. Also use wormwood annua. Also see Perniciosia)—4, 20, 28, 222, 550, 713, 880, 930, 1032, 1433, 1444, 1445, 455, 743; Malaria_1—20, 555; Malaria_2—20, 28, 787, 880; Malaria_3 (all freqs 5 to 15 min each)—880, 800, 787, 728, 20; Malaria_Falciparum_1—1518, 1348, 1473, 1002, 1019; Malassezia_furfur (causes tinea versicolor. See also Fungus general)—222, 225, 491, 616, 700; Mamma_fibromatosis (use Breast, fibroid cysts); Mange_follicular—253, 693; Mannan—961, 661; Marsh_elder—474; Mastitis (an inflamed breast usually caused by bacterial infection)—654, 698; Mastoiditis (inflammation of the bony structure of the head in the region of the ears below the eyes)—287; Measles—727, 787, 880, 342, 442, 443, 467, 520, 521, 552, 1489, 745, 757, 763, 712; Measles_HC (antigen)—18471.00, 919.62; Measles_rubella (German or 3 day measles)—431, 459, 510, 517, 796, 967, 368, 734, 772; Measles_rubella_secondary—727, 787, 880; Measles_rubella_vaccine—429, 459, 832, 926, 505; Measles_rubeola (9 day measles)—342, 467, 520, 784, 787, 962, 1489; Measles_vaccine—214, 725, 747, 783, 962; Measles_vaccine (try for autism)—1489, 962, 880, 787, 783, 763, 757, 747, 745, 727, 725, 712, 552, 521, 467, 443, 342, 214; Medorrhinum (homeopathic nosode for urethral discharge. See also Vaginosis.)—230, 442, 554, 843, 854, 1700, 1880, 2222, 602; Menieres_1—8.8, 8.9, 9.0, Menieres_2—456.6, 723, 880; Menieres_disease (auditory vertigo associated with deafness and tinnitus. Use 5000 for 16 min, 782 for 26 min, others for 7 min. Also use General_antiseptic)—1550, 802, 880, 787, 727, 465, 428, 33, 329, 5000, 1130, 782, 0; Meningcoccus_Virus—720; Meningioma (a benign, slow-growing tumor of the membranes that envelop the brain and spinal cord)—446, 535, 537; Meningitis (inflammation of the membranes that envelop the brain and spinal cord. Bacterial:

also use Streptococcus pneumoniae, Influenza haemophilus type B and see Listeriose and Leptospirosis. Viral: also use Echo, Coxsackie, and Polio)—5000, 1795, 1422, 1044, 928, 822, 764, 733, 720, 517, 427, 423, 322, 20; Meningitis_secondary—130, 676, 677, 507, Meningitis_tertiary—1550, 802, 880, 832, 787, 727, 650, 625, 600, 465, 444, 1865, 125, 95, 72, 20, 428, 660; Menopause_symptoms_TR—880, 787, 727, 5.5; Menstrual_problems (douche with plain water first)—880, 1550, 802, 787, 727, 465, 20, Mental_concentration—10000, 7.82; Mental_disorders (general aid; especially if toxins are the cause. Also use mineral supplement)—522, 146, 10000, 125, 95, 72, 20, 4.9, 428. 550, 802, 7.83; Mercury_toxicity_v—47, 48. 49, 75, Methotrexate_toxicity—584; Microsporum_audouini (a fungus which commonly causes ringworm of the scalp and other areas)—422, 831, 1222, 285; Microsporum_canis (a fungus causing ringworm in cats, dogs, and children)—347, 970, 1644, 402, 650; Migraine (also use Parasites strongyloides and general sets)—10; Mold (see also specific types)—222, 242, 523, 565, 592, 623, 745, 933, 1130, 1155, 1333, 1833, 4442; Mold_and_fungus_general_v (see also specific types)—4442, 2411, 1833, 1823, 1333, 1155, 1130, 1016, 942, 933, 886, 880, 866, 784, 774, 766, 745, 743, 728, 623, 623, 594, 592, 565, 555, 524, 512, 464, 414, 374, 344, 337, 321, 254, 242, 222, 158, 132; Moles—20, 120, 177, 600, 625, 626, 650, 659, 660, 728, 784, 880, 464; Moles_1 (use 761.7 for 15 min, others for 10 min)—761.7, 650, 625, 600, 76.2; Mononucleosis (use Epstein Barr N′inis); Monotospora_languinosa (homeopathic remedy for fungal allergen)—788; Morbillinum—467, 520, 1489; Morbus_Parkinson (use Parkinson's disease); Morgan (bact)—778, 784, 787, 788, 988, 726; Morgellons_disease_TR—5858.25, 5856.375, 4271.25, 4264, 330, 10000, 7344, 5000, 1550, 1234, 740, 880, 835, 787, 727, 160, 500, 1600, 5611, 4014, 3448, 3347, 3176, 2929, 2867, 2855, 2791, 2720, 2489, 2180, 1862, 1488, 880, 800, 787, 728, 665, 464, 432, 304, 120, 30, 20.8, 920, 2016.625; Motion_sickness (also see nausea)—10000, 5000, 648, 624, 600, 465, 440, 648, 444, 1865, 522, 190, 146, 125, 95.72, 20; Mouth_eruptions_herpes (use Herpes and Herpes Simplex I); Mouth_eruptions_white_patches (use leukoplakia); Mucocutan_perniciosis—833, 667, 756; Mucor_mucedo (causes rot in fruit and baked goods and sometimes found on feet and skin)—612, 1000, 488, 766, 9788, 735; Mucor_plumbeus—361, 578, 785, 877; Mucor_racemosis (grows on decaying vegetation and bread and can cause ear infection)—310, 474, 875; Mucor_racemosis_secondary—473, 686, 871, 873, 876, 878, 887, 7768, 7976, 8788, 713, 729, 731, 751, 760, 778, 1200; Mucormycosis (use Zygomycosis); Mucous_membrane_inflammation_general—380; Mucoviscidosis use Cystic fibrosis); Multiple_sclerosis_1 (use 5000 for 30 min)—5000, 4992, 23570.5, 3767, 3056.9, 2720, 24669, 2252.8, 2213, 2189, 2088.59, 1883, 1875, 1550. 1331. 880, 854, 840, 802, 787, 784, 728, 690, 660, 624, 620, 524, 470, 464, 430, 421, 317, 304.6, 275, 253, 241.68, 235, 224, 218, 166, 143, 80.9, 20, Multiple_sclerosis_2—5000, 23570.5, 3056.9, 2466.9, 2252.8, 2088.59, 880, 787, 728, 690, 660, 304.6, 241.68, 80.9, 20; Multiple_sclerosis_3—2253, 2467, 2357, 2358, 242, 305, 2089, 3057, 81, 5000, 1550, 802, 880, 787, 728, 690, 660, 20; Multiple_sclerosis_4—3040, 5000, 2720, 10000, 470, 120, 240, 300, 328, 728, 880, 2005, 2006, 2007, 2008, 2009, 2010, 2011, 20; Multiple_sclerosis_5 (use 5000 for 30 min)—5000, 728, 166, 224, 317, 727, 787, 880, 218; Multiple_sclerosis_6 (use 5000 for 10 min)—10000, 6000, 5000, 3176, 2489, 3057, 2008, 2358, 1488, 2467, 3040, 880, 787, 800, 728, 665, 464, 242, 224, 304, 166, 120, 20; Multiple_sclerosis_brain—0.3, 140, 158, 166, 169, 190, 199; Multiple_sclerosis_brain_infection—2.6, 2.8, 5.1, 6.7, 7.2, 7.5, 11.3: Multiple_sclerosis_myelin_sheath_repair—4.4, 8, 9.4, 9.5, 9,8, 12.6, 13.3, 14.2, 14.4, 14.8, 225, 259, 280; Multiple_sclerosis_secondary—20, 143, 275, 430, 470, 524, 620, 624, 802, 840, 854, 1550, 2213, 5000, 728, 784, 880, 464; Multiple_sclerosis_stiff_legs—315.77; Multiple_sclerosis_tremor_or_twitch—470; Multiple_sclerosis_v (aspertame, mercury, benzene, lead, and toluene can cause same symptoms. Also use Chlamydia General and Herpes type 6 and see ALS sets, Herpes general, Blastocystis hominus, Parasites flukes, Shigella, Nocardia, and Herpes zoster)—2145, 938, 862, 841 741, 739, 682, 660, 572, 532, 520, 477, 442, 433, 344, 343, 342, 338, 324, 322, 253, 242, 112; Mumps (acute viral inflammation of the saliva glands. See also Coxsackie)—152, 242, 642, 674, 922; Mumps_HC (antigen)—19018.66, 946.88; Mumps_secondary—190, 235, 516, 1243, 1660, 2630, 3142, 9667, 729, 741, 759, 761, 1170; Mumps_tertiary—10000, 727, 2720, 2489, 2127, 2008, 428, 880, 787, 727, 20; Mumps_vaccine—273, 551, 711, 730, 14.19; Muscle_tonic—20, 120, 240, 300, 304, 324, 328, 728, 880, 5000, 10000; Muscles to relax—965, 20, 120, 240, 760, 6.8, 6000, 304; Muscular_dystrophy (disorder characterized by weakness and progressive wasting of skeletal muscles despite no concomitant wasting of nerve tissue. Aspartame, mercury, benzene, lead, and toluene can cause same symptoms. Also use Parasites flukes and see Parasites general, ALS, MS sets)—153, 522, 146, 880, 787, 727; Muscular_pain_and_injury (also see Pain and General antiseptic)—2720, 6000, 324, 320, 250, 240, 160, 125, 80, 40, 20, 10, 5.8, 2.5, 1.5, 1.2, 1, 0.5; Mycobacterium_phlei_HC—20412.70, 1016.29; Mycobacterium_tuberculosis_HC (causes tuberculosis)—21508.01, 1070.82; Mycogone_fungoides—488, 532, 662, 764, 852, 1444; Mycogone_fungoides_secondary—328, 367, 490, 491, 495, 496, 628, 678, 761, 766, 768, 1055, 1074, 9979, 709, 714, 729, 746, 757; Mycogone_spp (homeopathic allergenic preparation based on fungus)—371, 446, 1123, 748; Mycoplasma_fermentans (May be a factor in ALS, chronic fatigue, alzheimer's, parkinson's, and MS)—2900, 878,2, 880.2, 864, 790, 706.7, 690, 610, 484, 986, 644, 254; Mycoplasma_general (can be useful for lung, sinus, and other problems which do not respond to other frequencies)—7344, 2950, 2900, 2842, 1147, 1113, 1067, 1062, 1045, 969.9, 880.2, 878.2, 865, 829.3, 800.4, 790, 783.6, 779.9, 706.7, 690.7, 690, 686.6. 684.1, 679.2, 673.9, 664, 644, 610, 484, 254; Mycoplasma_HC—16106.12, 17226.33, 801.88, 857.65, Mycoplasma_pneumonia—688, 975, 777, 2688, 660, 709.2, 2838.5; Mycoplasma_pulmonis—2404.2, 601, 300.5, 150.3, 75.1; Mycoplasma_salivarium_1 (run each for 18 min after building up)—253, 279, 420, 453, 761, 832; Mycosis_fungoides (a form of skin cancer resembling eczema)—532, 662, 678, 852, 1444; Myocarditis_narbe (narbe means scar)—279, 761; Myocardins_necrose (homeopathic remedy from heart cells that died as a result of inadequate blood flow to them. See also Circulatory stasis)—706, 789; Myoma (a benign tumor on the uterus)—253, 420, 453, 832; Myositis (involves progressive muscle weakness)—120, 122, 125, 129, 1124, 1169, 762; Myxosoma_HC—20611.85, 1026.20; Naegleria_fowleri_HC—18022.92, 897.31;

Nanobacter (Nanobacterium sanguineum, a nanobacteria which can be found in some calcium deposits in the arteries, kidneys, gall bladder, muscle and joints as well as in increased numbers in those with “autoimmune” diseases such as lupus, psoriasis, scleroderma, and similar disorders)—634, 317, 1268, 1902; Nanobacteria_2—6771.59, 6772.13, 6749, 6773.44, 6772.29, 6725.50, 5965.19, 5198.33, 5543.65, 5631.24, 9916.73, 8798.81, 8661.95, 4628.34, 4128.50, 2931.45, 2208.53, 2100.67; Nanobacteria3—13543.18, 13543.89, 13544.26, 13544.49, 13546.48, 13546.88, 13544.59, 13545.21, 13546.95, 11930.39, 10396.66, 9916.73, 8798.81, 8661.95, 4628.34, 4128.50, 2931.45, 2208.53, 2100.67; Nanobacteria_sanguineum_TR—7635.45, 6653.86, 6346.71, 5631.24, 5543.65, 2962.14, 2642.24, 1876.13, 1413.46, 1344.43, 1902, 317; Nasal infection (use Sinusitis and/or Rhinitis); Nasal_polyp (benign growth inside the nasal passage)—542, 1436; Nasturtium (a healing herb)—143; Nausea_and_cramping (also see Parasites, enterobiasis, roundworms, and Parasites, general for chronic conditions)—72, 95, 190, 880, 832, 787, 727, 20, 4.9, Nematodes (Use Parasites, nematodes); Neoplasms (Use cancer); Nephritis (kidney inflammatory disease. Also treat for stones and crystals supplementally, or nephrosis non inflammatory kidney disease)—1550, 274, 423, 636, 688, 880, 787, 727, 10, 20, 10000, 40, 73, 465, 3000; Nerve_disorders_and_neuropathy—10000, 3176, 2720, 2489, 2170, 2112, 1800, 1600, 1550, 880, 802, 787, 727, 660, 650, 625, 600, 440, 125, 95, 72, 20, 7.83, 3; Nerves_healing—200, 10, 7.64; Nervousness_Prozac_agitation (akathisia. See also Relaxation)—3, 7.83; Neuralgia (a painful disorder of the nervous system)—833, 3.9, 10000; Neuralgia_intercostal—802; Neuralgia_trigeminal—880, Neurosis—28; Neurospora_sitophila—705, 878; Nigrospora_spp—302, 350, 764; Nocardia_asteroides (the microorganism causing Nocardiosis, an infectious pulmonary disease characterized by abscesses in the lungs. Also see Streptothrix,)—228, 231, 237, 694, 719, 747, 887, 2890, Nocardia_asteroides_HC (found in Parkinson's disease)—17679.39, 880.20; Nogier_frequencies—1.14, 2.28, 4,56, 9.125, 18.25, 36.5, 73, 146, 292, 584, 1168, 2:336, 4672; Nose_infection_congestion (use Sinusitis and Rhinitis); Numbness (also see Circulatory stasis)—10000, 2720, 2489, 2170, 1800, 1600, 1550, 802, 880, 787, 727, 650, 625, 600, 440, 660;

Oat_smut—806; Oligodena—853; Oospora—9599, 5346; Oral_lesions (chronic cases will always return until metal dental bit is replaced with uranium free porcelain. See also herpes simplex I and use: Stomatitis)—2720, 2489, 2008, 1800, 1600, 1550, 802, 880, 787, 727, 465, 444, 522, 146; Orchitis (inflammation of testes due to TB, mumps, gonorrhea, cancer, bacteria, etc. See also causative condition if known)—2720, 2489, 2170, 2127, 2008, 1800, 1600, 1550, 802, 1500, 880, 832, 787, 776, 727, 690, 666, 650, 625, 600, 125, 95, 72, 20, 9; Orchitis_secondary—727, 787, 880, 10000; Ornithosis (Psittacosis or Parrot Fever; an infectious pneumonia transmitted by certain birds)—233, 331, 332, 583, 859, 1217; Osteitis (bone inflammation)—770; Osteoarthritis (use also Arthritis)—962, 1500, 770; Osteoitis—2,65; Osteomyelitis—2720, 2489, 2170, 2127, 2008, 1800, 1600, 1550, 802, 1500, 880, 832, 787, 776, 727, 690, 666; Osteomyelosclerosis (marrow replacement by bone in response to low-grade infection)—79, 330; Osteosinusitis_max—243, 327; Ostitis—770, 743, 736, 724; Otitis_externa (outer ear infection. Also see Pseudomonas aeruginosa)—727, 787, 880, 174, 482, 5311; Otitis_medinum (middle ear swelling and or infection and fever. Use also strep pneumonia if no immediate benefits. Use 880, 720, and 1550 for 10 min, others for 5 min)—683, 688, 776, 766, 316, 784, 786, 125, 802, 72, 522, 440, 880, 720, 1550; Otosclerosis—9.19; Ovarian cyst—567, 982, 711; Ovarian_disorders_general—2720, 2489, 2170, 2127, 2008, 1800, 1600, 1550, 1500, 982, 880, 832, 802, 787, 776, 727, 711, 690, 666, 650, 625, 600, 567, 465, 444, 26, 20; Ovarian_elimination_stimulation—20, 800, 1550; Ovum—752; Ox_bile (the homeopathic remedy derived from it)—672; Ozaena—184, 222, 439;

Pain_acute—3000, 95, 10000, 1550, 802, 880, 787, 727, 690, 666; Pain_general (Also look under name of condition causing pain)—3000, 2720, 95, 666, 80, 40; Pain_of_cancer—3000, 95, 2127, 2008, 727, 690, 666; Pain of infection—3000, 95, 880, 1550, 802, 787, 776, 727, 4.9; Pain relief—304, 6000, 3000, 666, 80; Palsy_general—10000; Pancreas—440, 464, 600, 624, 648, 1552, 727, 787, 880; Pancreatic_insufficiency—20, 250, 650, 625, 600, 465, 444, 26, 2720, 2489, 2170, 2127, 2008, 1800, 1600, 1550, 802, 1500, 880, 832, 787, 776, 727, 690, 666, 20; Papilloma_kidney (small, usually benign growth on a kidney)—110, 148, 264, 634, 767, 848, 917, 760, 762, 1102; Papilloma_virus (causes warts and benign tumors having a branch or stalk and in some cases white patches known as leukoplakia, 874*, 907*)—67265, 64734, 16970, 9609, 9258, 5657, 1051, 1011, 907, 874, 767, 489, 466, 404, 265, 110, 45; Papilloma_virus_cervix_HC (smear)—20128.91, 1002.16; Papilloma_virus_plantar_wart_HC—20163.76, 1003; Papilloma_virus_wart_HC—20263.34, 1008.85; Paraceli—232; Paradontose—424, 1552; Paralysis_nonspastic—10000, 880, 787, 776, 727, 650, 625, 600, 444, 1865, 125, 95, 72, 20, 9.19, 8.25; Paralysis_spastic—10000, 880, 787, 776, 727, 650, 625, 600, 444, 1865, 125, 95, 72, 20, 7.69; Paramecium_caudatum—4500, 1150, 2298; Paramyxovirus (experimental!, types avian 6 and tupaia)—8920.5, 8089.4, 4460.2, 4044.7, 2230.1, 2022.3, 1115.1, 1011.2, 557.5, 505.6, 278.8, 252.8, 139.4, 126.4; Parasites_ancylostoma_braziliense_HC (Dog and cat hookworm, the larva of which is the most common cause of cutaneous larva migrans aka creeping eruption. Also see Parasites hookworm)—19964.61, 993.98; Parasites_ancylostoma_caninum_HC—19914.83, 991.50, 19566.32, 974.15, 19217.81, 956.80, Parasites ascaris (152*)—1.52, 442, 8146, 751, 1146, 797; Parasites_ascaris_HC (larvae in lung)—20313.12, 1011.33; Parasites_ascaris megalocephala_HC—20:313.12, 1011.33; Parasites_capillaria_hepatica—21308.87, 1060.91; Parasites_clonorchis_sinensis_HC—21259.08, 1058,4:3; Parasites_cryptocotyle_lingua—20611.85, 1026.20; Parasites_dirofilaria_immitis_HC (dog heartworm)—20362.91, 1013,81; Parasites_echinoparyphium_recurvatum_HC—20960.36, 1043.55; Parasites_echinostoma_revolutum_HC—21308.87, 1060.91; Parasites_enterobiasis (pinworms; intestinal worms which cause itching of the anal and perianal areas)—20, 112, 120, 773, 826, 827, 835, 4152; Parasites_enterobius_vermicularis_HC—21059.93, 1048.51; Parasites_eurytrema_pancreaticum_HC—20960.36, 104:3.55, Parasites_fasciola_hepatica_HC—21159.50, 1053.47; Parasites_fasciola_hepatica_cercariac_HC—21259.08, 1058,43; Parasites_fasciola_hepatick_eggs_HC—21159.50, 1053,47; Parasites_fasciola_hepatica_miracidia_HC—21059,93, 1048.51; Parasites_fasciola_hepatica_rediae_HC—21159.50, 1053.47; Parasites_fasciolopsis_buskii_adult_HC—21607.59, 1075.78; Parasites_fasciolopsis_buskil_eggs_HC—21607,59, 1075.78; Parasites_fasciolopsis_cercariae_HC—21607,59, 1075.78; Parasites_fasciolopsis_miracidia_HC—21607.59, 1075.78; Parasites_fasciolopsis_rediae_HC—21508.01, 1070.82; Parasites_filariose (worms in blood and organs of mammals, larvae passed from biting insects)—112, 120, 332, 753; Parasites_fischoedrius_elongatus_HC—22005.88, 1095.61; Parasites_flukes_blood—847, 867, 329, 419, 635, 7391, 5516, 9889; Parasites_flukes_general (pancreatic, liver, and intestinal)—6766, 6672, 6641, 6578, 2150, 2128, 2082, 2013, 2008, 2003, 2000, 1850, 945, 854, 846, 830, 763, 676, 651, 524, 435, 275, 142; Parasites_flukes_general_short_set—524, 854, 651; Parasites_flukes_intestinal (2127/2128*)—524, 651, 676, 844, 848, 854, 2128, 2008, 2084, 2150, 6766; Parasites_flukes_liver—143, 275, 676, 763, 238, 6641, 6672; Parasites_flukes_lymph—10050, 157; Parasites_flukes_pancreatic_1—1850, 2000, 2003, 2008, 2013, 2050, 2080, 6578; Parasites_flukes_sheep_liver—826, 830, 834; Parasites_follicular_mange—253, 693, 701, 774; Parasites_gastrothylax elongatus_HC—22653.12, 1127.83; Parasites_general_1—4412, 2400, 2112, 1862, 1550, 800, 732, 728, 712, 688, 676, 644, 422, 128, 120; Parasites_general_2—10000, 3176, 1998, 1865, 1840, 880, 800, 780, 770, 740, 728, 727, 690, 665, 660, 465, 444, 440, 125, 120, 95, 80. 72, 47; Parasites_general-alternative_v—4122, 1522, 967, 942, 854, 829, 827, 749, 741, 732, 633, 605, 604, 591, 524, 422, 411, 344, 172, 102; Parasites_general_comprehensive—10000, 5000, 4412, 2720, 2400, 2112, 1864, 1550, 1360, 880, 854, 800, 784, 751, 732, 728, 712, 688, 651, 644, 524, 465, 442, 422, 334, 240, 152, 128, 125, 120, 112, 96, 72, 64, 20; Parasites_general_shortset—20, 64, 72, 96, 112, 120, 152, 651, 732, 1360, 2720, 10000; Parasites_gen_custom2_TR (sweep 2000 to 2008 by 1 dwell 360)—6578, 2000, 831, 2000, 2008, 2520, 689, 750, 880, 650, 187; Parasites_giardia—5768, 5429, 4334, 2163, 2018, 1442, 829, 812, 721, 407, 334; Parasites_giardia_lablia_HC—21109.72, 1050.99; Parasites_gyrodactylus_HC—18919.09, 941.93; Parasites_haemonchus_contortus_HC—19566.32, 974.15; Parasites_heartworms—543, 2322, 200, 535, 1077, 799, 728; Parasites_helminthsporiim) (worm eggs)—793. 969, 164, 5243; Parasites_hookworm—6.8, 440, 2008, 6436, 5868; Parasites_leishmania_braziliensis—787; Parasites_leishmania_braziliensis_HC—20064.19, 998.94; Parasites_leishmania_donovani—525, 781; Parasites_leishmania_donovani_HC—19914.83, 9911.50; ParasitesleishmaniamexicanaHC!—20014.40, 996.46: Parasites_leishmania_tropica—791: Parasites_leishmania_tropica_HC—20163,76, 1003.89; Parasites_loa_loa_HC—17973.13, 894.83; Parasites_macracanthorhynchus_HC—21906.31, 1090,65; Parasites_metagonimus Yokogawai_HC—21906.31, 1090,65; Parasites_nematode—771; Parasites_onchocerca_volvulus—HC (tumor)—21906.31, 1090.65: Parasites_paragonimus_Westermanil_HC—22503.75, 1120.40, 22254.82, 1108,00; Parasites_passalurus_ambigutis_HC—21956.10, 1093,13, 21756,95, 1083.21; Parasites_pinworm (use Parasites, enterobiasis); Parasites_roundworms_comp—7159, 5897, 4412, 4152, 3212, 2720, 2322, 1372, 1113, 1077, 1054, 942, 835, 827, 826, 822, 799, 776, 773, 772, 753, 752, 749, 746, 738, 732, 728, 722, 721, 698, 688, 650, 543, 541, 535, 422, 380, 332, 240, 200, 152, 128, 120, 112, 104, 101, 20; Parasites_roundworms_general—7159, 5897, 4412, 4152, 3212, 2720, 942, 835, 827, 772, 732, 721, 688, 650, 543, 422, 332, 240, 152, 128, 120, 112, 104, 20; Parasites_roundworms_general_short_set—128, 152, 240, 422, 650, 688; Parasites_roundworms_flatworms_TR (use when there is chronic pain from these. 40 min each with converge 1 0,03125 or 1 0.03333)—6187.5, 6468.8, 5050; Parasites_scabies (follicular mange which is contagious dermatitis found in many animals that is caused by mites and in which the principle activity is at the hair follicles. Use 90, 94, 98, 102, 106, 110, 253, 693 for 10 min. Scan 90 to 110 on lesser frequency intervals if needed. Also, rub skin with olive oil, let sit, then rinse with thyme tea)—920. 1436, 2871, 5742, 90, 94, 98, 102, 106, 110, 253, 693; Parasites_schistosoma_haematobium (blood flukes)—847, 867, 635; Parasites_schistosoma_haematobium_HC—23549.28, 1172.45; Parasites_schistosoma_mansoni (blood fluke which can cause symptoms identical to hepatitis C)—329, 9889; Parasites_stephanurus_dentalus (ova)—22951.84, 1142.70; Parasites_strongyloides (threadworm, genus of roundworms)—332, 422, 721, 732, 749, 942, 3212, 4412; Parasites_strongyloides_HC (filariform larva)—19914.83, 991,50; Parasites_strongyloides_secondary—380, 698, 752, 776, 722, 738, 746, 1113; Parasites_taenia (use Parasites, tapeworms); Parasites_tapeworms (if any of these frequencies are felt strongly, also use a good herbal antiparasitic regimen plus CoQ10 in large amounts, 187*, 5522*)—164, 187, 453, 523, 542, 623, 843, 854, 1223, 803, 1360, 3032, 5522; Parasites_tapeworms_echinococcinum (tapeworms found in dogs, wolves, cats, and rodents that can infect man, 5522*)—164, 453, 542, 623, 5522; Parasites_tapeworms_secondary—142, 187, 624, 662; Parasites_threadworms (use Parasites, strongyloides); Parasites_trichinella_spiralis_HC (found in muscle)—20138.87, 1002.66; Parasites_trichinosis—101, 541, 822, 1054, 1372; Parasites_trichomonas_vaginalis_HC—18968.87, 944.40; Parasites_trichuris_sp_HC (male)—20213.55, 1006.37; Parasites_trypanosoma_brucel_HC—21358.65, 1063.38; Parasites_trypanosoma_cruzi_HC (brain tissue)—23051.41, 1147.66; Parasites_trypanosoma_equiperdum_HC—22055.67, 1098.09, 22005.88, 1095.61, 21707.16, 1080.74; Parasites_trypanosoma gambiense_HC—19715.68, 981.59; Parasites_trypanosoma_lewisi_HC (blood smear)—21159.50, 1053.47; Parasites_trypanosoma_rhodesiense_TR—21209.29, 1055.95; Parasites_turbatrix—104; Parasites_urocleidus_HC—22254.82, 1108.00; Paresis—9.4; Paresthesia 5.5; Parkinson_tremor_temporary_relief (use 6000 for 10 nun)—6000, 470, 130, 169; Parkinsons_disease (a slowly progressive, degenerative, neurologic disorder, also use Mycoplasma fermentans, try Chlamydia pneumoniae, and see Nocardia asteroides freqs. Use 33 for 30 min, 33*)—470, 693, 813, 1.1, 5000, 6000, 1131, 33; Parkinsons_v—4334, 1422, 871, 840, 827, 813, 744, 742, 733, 658, 611, 577, 569, 531, 524, 442, 314, 310, 172, 134; Parrot_Fever (use Ornithosis): Parvovirus_canine—637, 185, 323, 562, 613, 622, 1000, 4027; Parvovirus_canine_mutant_strain—323, 514; Paryovirus_canine_type_B—323, 535, 613, 755; Parvovirus_new_strain—2257, 488; Pasteurella_combination (bacterial diseases spread by animal bites)—323, 913, 694; Pelvic_inflammatory_disease—2950, 2976, 2720, 2489, 2170, 2127, 2008, 1800, 1600, 1550, 1488, 802, 787, 776, 727, 690, 666, 650, 625, 600, 465, 444, 522, 95, 72, 450, 428, Pemphigus (rare, autoimmune skin disorders characterized by blisters in the outer layer of skin and mucous membranes)—694, 893, 665; Penicillium_chyrosogenium—129, 249, 344, 967; Penicillium_chyrosogenium_secondary—345, 688, 868, 1070, 2411, 728, 764, 765; Penicillium_notatum—334, 629; Penicillium_notatum_secondary—321, 550, 555, 556, 558, 560, 562, 566, 572, 644, 825, 922, 942, 4870, 7780, 412, 715; Penicillium_rubrum—332, 457, 460, 462, 766, 1015, 1018; Pennyroyal (an herb)—772: Pepto_streptococcus (see also other Strep sets)—201, 629, Pericarditis (NOTE, Cardiac conditions are inherently unstable. Therefore, we cannot recommend the use of frequencies on humans, even outside U.S. jurisdiction. These frequencies are for animal research only.)—2720, 2170, 1600, 880, 1550, 802, 787, 727, 625, 125, 95, 72, 20; Periodontal_disease (see also osteo and dental infection freqs)—47.5, 1800, 1600, 650, 625, 600, 880, 787, 776, 727; Peritonitis—727, 787, 880; Perniciosia (scilicet malaria, extremely severe form of malaria)—232, 622, 822, 4211; Perniosis (chillbains. A disorder of the blood vessels caused by prolonged exposure to cold and characterized by skin lesions on the lower legs, hands, toes, feet, ears and face)—20, 232, 622, 822, 4211, 2112; Pertussis (whooping cough)—526, 765, 46, 284, 9101, 697, 906; Pertussis_secondary—880, 832, 802, 787, 776, 727, 1234, 7344; Pesticide_detox (see also Liver and Kidney support, Circulatory and Lymph stasis freqs)—73, 26, 6, 1; Phaqocyross_stimulation—20, 125, 727, 787, 880; Pharyngitis (inflammation of pharynx, can cause chronic sore throat, halitosis, and pharyngeal ulcers. Also see Sore throat, Halitosis)—440, 380, 1600, 1550, 802, 776, 784, 880, 727, 20, 522, 146; Phlebitis (vein inflammation)—1500, 776; Phoma_Destructiva (homeopathic)—163, 815, 621; Pineal_gland_stimulation—20; Pingueculum (conjunctival tumors. Ping_eculum is the medical term for a common white or yellow raised area on the conjunctiva; mostly a hint to a neurolymphatic condition.); Plague (use Bubonic plague); Planetary_orbits (brainwave freqs, from mercury to pinto)—141.27, 221.23, 272.2, 144.72, 183.58, 295.7, 414.7, 422.8, 280.5; Plantaris—2008; Plasmacytoma (use Cancer_plasmacytoma); Pleurisy (Inflammation of the lung membrane and abdominal lining)—1550, 802, 880, 787, 776, 727, 125, 95, 72, 20, 444, 1865, 450; Pneumococcus (use Streptococcus pneumoniae); Pneumocystis_camii (fungus which causes pneumonia usually developing in the immune suppressed or in infants)—204, 340, 742; Pneumonia_1—10346.56, 10976.38, 5045.03, 5548.59, 5549.22, 5554.69, 5554.84, 5558.75, 5560.78, 5562.5, 6752.01, 7118.2, 7255, 7284.06, 7414.28, 7631.09, 7632.66, 7667.38, 7676.94, 8045.81, 8041.5, 10334.06, 8082.59, 8305.19, 8911.25, 9113.5, 9141.5, 9393.13, 6654.69; Pneumonia_2—986, 987, 988; Pneumonia (inflammation of bronchii and lungs)—550, 802, 880, 787, 776, 727, 452, 1474, 578; Pneumonia_general (see also Pneumonia klebsiella, Pneumonia mycoplasma, Pneumonia bronchial, Pneumonocystis carinii, Streptococcus pneumoniae. If no or slow results, try Mycoplasma General set.)—7660, 7344, 5000, 3702, 3672, 2688, 1862, 1550, 1238, 1234, 1200, 975, 880, 802, 787, 780, 778, 776, 774, 772, 770, 768, 766, 727, 688, 683, 660, 450, 412, 352, 20; Pneumonia_general_v—6007, 5423, 5421, 5420, 5419, 2688, 2581, 2356, 967, 877, 838, 765, 748, 746, 568, 542, 532, 522, 520, 440; Pneumonia_mycoplasma (a contagious pneumonia of children and young adults. See also Mycoplasma General)—688, 975, 777, 2688, 660, 709.2, 2838.5; Pneumonia_walking (use Pneumonia mycoplasma); Pneumoniae_klebsiella (causes an acute, bacterial pneumonia)—840, 818, 783, 779, 776, 766, 765, 746, 413, 412; Pneumoniae_klebsiella—19964.61, 20860.78, 1038.60, 993.98; Pneumovirus (causes bronchiolitis and pneumonia in infants, use Respiratory syncitial virus); Polio (also known as poliomyelitis)—135, 283, 742, 776, 1500, 2632, 1850; Polio_secondary_complications—1550, 802, 428, 1500, 880, 787, 727; Polyarthritis—512; Polyp_general—522, 146, 2720, 2489, 2170, 2127, 2008, 1800, 1600, 727, 690, 666, 650, 625, 600, 465, 444, 20; Polyp_nasal—542, 1436; Polyp_uterine—689; Porphyria (several rare disorders of the nervous system and skin)—698, 780; Postsurgery_detoxification (also use saunas and electrolyte replacement)—522, 146; Proctitis (use Chlamydia trachomatis); Propionibacterium_acnes (may be secondary cause of some prostate cancers. Sweep from 5996.1 to 6078.1 by 0.03125 dwell 1 pulse 64 75 plus 6046.9 for 6 min)—60469; Propionibacterium_acnes _BC—19267.60, 959.28; Prostate_adenominum—442, 688, 1875, 748, 766: Prostate_enlarged (see Prostatitis and other prostate sets and Strep general)—2250, 2128, 2050, 920, 690, 666; Prostate_hyperplasia (use Prostate_enlarged); Prostate_problems_general (see also Strep sets and practice metals avoidance)—2720, 2128, 2008, 880, 802, 787, 728, 727, 690, 666, 465, 408, 125, 95.72, 20, 9; Prostatitis (benign prostate tumor or infection)—100, 410, 522, 146, 2720, 2050, 2489, 2170, 2127, 2008, 1550, 802, 787, 776, 727, 690, 666, 465, 125, 95, 72, 20, 444, 522, 9,1; Prostatitis 1 (use 2050 and 2250 for 15 min)—2050, 2250; Proteus—424, 434, 834; Proteus_mirabilis_HC—16131.01, 803.12; Proteus_vulgaris_HC (urinary tract pathogen)—20562.06, 16728.45, 16330.16, 1023.72, 832,86, 813,03; Pruritis (use Itching); Pseudomonas_aeruginosa (bacteria causing blue pus infections found in wounds, burns, and the urinary tract, et al)—174, 482, 785, 1132, 5311, 3965, 405, 633, 191, 178, 731, 6646, 178, 739.8, 2959.4; Pseudomonas_aeruginosa_HC (causes infection in open wounds)—16579.09, 825.42; Pseudomonas_gen—6646, 5311, 3965, 2959.4, 1273, 1132, 986, 875, 857, 785, 774, 743, 739.8, 737, 731, 687, 633, 501, 482, 437, 405, 191, 178, 174; Pseudomonas_mallei (also called glanders bacillus. Causes glanders, a blue pus infection of the respiratory system and mouth, and transmitted occasionally to man by equine.)—986, 737, 687, 857, 875, 1273, 501, 743, 774; Pseudomonas_pyocyanea—437; Psittacosis (use Ornithosis); Psoriasis (also use Hypothyroid set and see General Parasite, ascaris, and other roundworm freqs)—2720, 2489, 2180, 2170, 2128, 2008, 1552, 880, 800, 786, 728, 664, 304, 152, 112, 104, 100, 96, 64, 60; Psoriasis_ankylosing_spondylitis—3000, 95, 1550, 802, 880, 787, 776, 727, 650, 625, 600, 28, 1.2, 10, 35, 28, 7.69, 110, 100, 60, 428, 680; Psorinum—786, 767; Ptosis (Eyelid droop. Also use Parasite general, ascaris, and roundworm); Pullidaria_pullulans (allergy remedy)—432, 873, 1364, 684, 739, 750; Pulmonary_fibrosis (use also Parasite, roundworm frequencies and see General Antiseptic)—27.5, 220, 410; Pyelitis_proteus (bacteria commonly found in hospital-borne conditions)—434, 594, 776; Pyocyaneus (homeopathic nosode for Pseudomonas pyocyanea)—437; Pyoderma (or Pyoderma Gangrenosum; a rare skin disorder of unknown cause. Small pustules develop into large ulcers at various sites on the body. Also see Parasites general and General antiseptic)—123, 132, 967, 974, 1556, 1489, 663; Pyorrhea (infection of periodontium causing inflammation of gums and bone loss. Although pyorrhea may be controlled or eliminated with application, infection will always return whenever subject experiences stress or poor diet, until mercury inlays aka ‘silver’ fillings and nickel alloy which is stainless steel, dental appliances are replaced with porcelain that does not contain uranium and infected root canals and sockets are treated. See also Dental, Dental foci, Stomatitis, Gingivitis, Toothache)—2720, 2489, 2008, 1800, 1600, 1550, 802, 880, 787, 776, 766, 727, 465, 444, 522, 146; Pyrogenium_62 (general homeopathic remedy for pus)—151, 429, 594, 622, 872, 497, 498; Pyrogenium_ex_ovo—231, 1244, 1210, 1216; Pyrogenium_fish—287, 304; Pyrogenium_mayo—688, 759, 1625, 727, 770; Pyrogenium_suis—341, 356, 673;

Q Fever (an infectious disease caused by contact with animals with the parasitic Rickettsia bacteria and Coxiella burnetii whose symptoms may include headache, fever, chills, and sweats. See also Typhoid fever and Rickettsia)—523, 1357, 607, 129, 632, 943, 1062, 549, 720, 726;

Rickettsia bacteria and Coxiella burnetii whose symptoms may include headache, fever, chills, and sweats. See also Typhoid fever and Rickettsia)—523, 1357, 607, 129, 632, 943, 1062, 549, 720, 726; Rabies (lyssinum)—20, 120, 547, 793; Radiation_burns (also see Regeneration and Healing)—727, 787, 880, 10000; Ragweed—473; Raynauds_disease—727, 20; Refractory_an—435; Regeneration_and_healing—47, 2720; Regeneration_and_healing_1—2720, 2,0, 20.5, 3,9, 4.0, 50.5, 6.3, 148, 7.0; Relaxation_to_produce—6000, 10, 7.83; Renal_calculi (use Kidney stones); Renal_excretory_insufficiency_diastolic_hypertensive (also see Kidney insufficiency)—9.2; Reproductive—335, 536, 622, 712; Respiratory_syncytial_virus—336, 712, 278; Respiratory_syncytial_virus_HC—18919.09, 941.93; Retrovirus_variants—2489, 465, 727, 787, 880, 448, 800, 10000; Rhesus_gravidatum—312, 322, 536, 684; Rheuma (watery discharge from nose or eyes)—952, 436, 595, 775; Rheumaticus—333, 376, 820; Rheumatism (also use Arthritis_rheumatoid)—10000, 776, 766, 262; Rheumatoid_arthritis (use Arthritis_rheumatoid); Rhinitis (ninny nose, also see Sinusitis)—20, 120, 1550, 802, 1500, 880, 787, 727, 465, 522, 146; Rhino_pneumonitis—185, 367, 820. 487; Rhizopus_nigricans—132, 327, 775, 659, 660; Rhodo_torula—833, 598, 778; Rhodococcus—124, 835, 432, 764, 337, 682, 720; Rickettsia (bacteria that are transmitted to man by lice, fleas, ticks, and mites. Can cause typhoid fever and Q fever.)—129, 632, 943, 1062, 549, 720, 726, 521, 2085, 4170; Ringworm (see Microsporum audouini, Microsporum canis, Trichophyton, and/or Epidermophyton. Use all freqs for 6 min)—5000, 800, 732, 442, 440, 422, 128, 120, 92, 76, 60, Roaches (may target a symbiot)—100; Rocky_Mountain_spotted_fever—375. 862, 943, 788, 468, 308; Rocky_Mtn_Spotted_Fever_and_Lyme_v—7989, 1590, 239, 846, 422, 417, 1455, 39975, 40439, 884. 797, 758, 693, 673, 577, 4870. 4880. 578, 128, 579; Rosacea (use Helicobacter pylori and take enzyme supplement), Roseola (use Herpes type 6); Rotifer—4500; Rubella (use Measles_rubella). Rubeola (use Measles_rubeola); Rukotick—6634, 285, 308;

Sacred_frequencies—11, 22, 33, 44, 55, 66, 70, 72, 77, 88, 99, 111, 144, 153, 222, 333, 444, 555, 666, 777, 888, 999, 1111, 1260, 3142; Salivary_gland_virus (human herpes type 5. Use Cytomegalovirus); Salmonella (1)—718.2, 713.3; Salmonella (can cause intestinal inflammation and infection, and contribute to flu in children)—1522, 718, 717, 713, 972, 664, 643; Salmonella_comp—8656, 7771, 6787, 1634, 1522, 1244, 972, 773, 762, 754, 752, 719, 718,2, 717, 713.3, 711, 707, 693, 664, 643, 546, 420, 165, 92, 59; Salmonella_enteriditis_HC (causes intestinal infection)—16379,95, 815.51; Salmonella_paratyphi_B—59, 92, 643, 707, 717, 719, 752, 972, 7771, 1244, 6787, 165, 711; Salmonella_paratyphi_HC—18321.64, 912.18; Salmonella_type_B—546, 1634; Salmonella_typhi (can cause typhoid fever)—420, 664, 8656, 7730; Salmonella_typhimurium—693, 754, 762; Salmonella_typhimurium_HC (can cause food poisoning, nervousness, apathy)—19168.02, 19217,81, 954.32, 956.80; Sanguinera (use Nanobacter); Sanguis_menst—591; Sarcocystis_HC—22503.75, 1120.40; Sarcoidosis (also see Cancer, lymphogranuloma)—2167, 2967, 3289; SARS_1—9563, 6157, 5513, 3735, 1559, 33566, 255616, 1556, 2286, 5763, 8015, 162, 563, 5613, 5235; SARS_2—597.68, 769.62, 689.14, 937.76, 779.5, 524.47, 1048.94, 499.25, 998.5, 778.12, 1143, 720.36, 1001.86, 648, 563, 701.6, 654.4; Scabies (use Parasites_scabies and Mange_follicular); Scarlet_fever—437, 880, 787, 727, 690, 666; Schistosoma (use Parasites, schistosoma); Schizophrenia_paranoid_1 (also see Tuberculosis)—802, 1500, 1550; Schumann_resonance (Earth frequencies which may be relaxing)—7.83, 14, 20, 26, 33, 39. 45; Schunan_B_Cell—322, 425, 561, 600, 620, 623, 780, 781, 950, 952, 1023, 1524, 1097, 1.100; Sciatica_1 (also use “Bone Builder with Boron” at double dose)—254, 464, 465, 866, 15, 25.4; Sciatica_2—782, 732, 860; Sciatica_schias—1550, 802, 880, 787, 727, 690, 666, 10; Scleroderma_v—4334, 42046, 44178, 49847, 55687, 67868, 75969, 35237, 54778, 44837, 39703, 31888, 34112, 36769, 42938, 38882, 48686, 30121, 64734, 44679, 70542, 48450; Sclerosis_lateral (degeneration of spinal cord resulting in spastic paraplegia)—254; Sedation_and_pain_relief—304, 6000; Sedative_effect (Reported use on bleeding, bruises, insomnia, sinusitis)—2.5; Seizure_1—329, 226, 953; Semperillium—1140; Serratia_marcescens_HC—17475.26, 870.04; Serum_schweinepest (swine fever or hog cholera)—503, 246, 604, 465; Sexual dysfunction men (see also Circulatory stasis, Orchitis)—9.39, 2112, 2127, 2008, 465, 880, 802, 787, 727, 690, 666, 124, 95, 73, 72, 20, 650, 625, 600; Shigella (can cause acute dysentery and diarrhea as well as infect netves, brain, and spinal cord chronically)—621, 762, 769, 770, 1550, 802, 832; Shigella_dysenteriae_HC—19421.39, 966.93; Shigella_flexneri_HC (causes depression)—19616.10, 976,63; Shigella_sonnnei_HC (invades tumors)—15832.29, 788.24; Shingles (use Herpes zoster); Silver—1590:3; Simian_virus_40 (SV40, also try for throat and sinus issues)—7635.45, 6653.86, 6346.71, 5631.24, 5543.65, 2962.14, 2642.24, 1876.13, 1413.46, 1344.43, 83173.3, 93806.5; Sinus_Bacteria (also use Sinusitis)—548: Sinusitis (sinus and lung infections can be caused by a large number of pathogens including viruses like RSV, adenovirus, parainfluenza, influenza A, influenza B, rhino virus, et al. Common bacteria like strep pneum, are always suspect when condition is chronic, as are fungi.)—952, 7411, 682, 320, 160; Sinusitis_1 (all freqs for 5 min)—1862, 1600, 1552, 1500, 880, 802, 784, 732, 728, 712, 683, 660, 548, 524, 464, 440, 400, 146, 120, 72, 20; Sinusitis 2—125, 160, 367, 472, 600, 615, 625. 650, 820, 952, 1150, 1520, 1865, 2000, 4392, 4400, 4412; Sinusitis_3—60, 95, 128, 225, 414, 427, 432, 456, 610, 614, 618, 1234, 2600, 5500, 304; Sinusitis_4—107, 160, 952, 942, 320, 741, 682, 1395; Sinusitis frontalis—952, 320, 682; Sinusitis_maxillaris—160, 741; Sinusitis_stubborn_TR—10000, 7344, 5000, 2950, 2900, 2650, 2600, 1550, 1234, 740, 880, 787, 727, 330, 165, 82.6, 41.75, 20.87, 30; Sleeping_sickness—120, 20; Slipped_discs (spasms from microbial toxins)—125, 880, 787, 728, 727, 95, 72, 20; Smallpox (also known as variola, an extremely contagious viral disease marked by fever, prostration, and a rash of small blisters)—3222, 2544, 2132, 1644, 1550, 876, 832, 802, 569, 542, 511, 476, 142; Smallpox_secondary—334, 360, 471, 647, 506, 711, 880, 787, 727, 20; Smegma—153, 180, 638; Sneezing—880, 787, 727, 683, 465, 146; Solfeggio_scale—963, 852, 741, 639. 528, 417, 396, 288, 174; Sore_throat (also see Pharyngitis, Strep general, Strep pyrogens, and Actinomyces israelii)—2720, 2489, 1800, 1600, 1550, 802, 885, 880, 875, 787, 776, 727, 46.5, 766; Sore_throat_1—34464, 14080, 4192, 3552, 2720, 2489, 2154, 1800, 1600, 1550, 802, 885, 880, 875, 787, 776, 727, 46.5, 766, 222, 262; Sore_throat comp—34464, 14080, 10000, 8450, 7880, 5004, 4192, 3552, 2720, 2502, 2489, 2154, 2111, 1998, 1840, 1800, 1600, 1560, 1550, 1214, 1000, 925, 885, 880, 875, 845, 802, 787, 776, 766, 747, 735, 727, 720, 690, 666, 660, 625,5, 624, 616, 590, 567, 522, 488, 465, 440, 432, 380, 340, 262, 222, 151, 146, 120, 46.5, 23; Sore_throat_TR—2720, 1800, 144, 452; Sorghum_smut (homeopathic preparation for an allergen)—294; Sorghum_syrup_HC—13791.02, 686.61; Spasms_muscle—6,8; Spastic_paresis—30.87, 48; Sphaerotilus_natans_HC—19466.74, 969.19; Spirillum_serpens_HC—18919.09, 941.93; Spleen_enlarged—35, 787, 3176; Spleen_secondary—10000, 2720, 2170, 1800, 1550, 880, 802, 727, 465, 20; Sporobolomyces—753, 700; Sporotrichum_prutinosum—584, 598, 687, 755, 715; Stammering—10000, 20, 6000, 7.83; Staph infection_1—943, 727, 643, 20; Staph_and_Strep_v—40887, 9646, 7160, 2431, 1902, 1109, 1060, 1050, 1010, 985, 958, 934, 786, 727, 718, 686, 643, 576, 563, 542, 453, 436, 423, 411, 333, 134, 128; Staphylococci_infection (see also other Staph freqs, 727*, 786*)—960, 727, 786, 453, 678, 674, 550, 1109, 424, 943. 1050, 643, 2600, 7160, 639, 1089, 8697; Staphylococcus_aureus (can cause boils, carbuncles, abscesses, tooth infection, heart disease, and infect tumors, 786*)—8697, 7270, 1050, 999, 943, 824.4, 787, 784, 745, 738, 728, 727, 647, 644, 555, 478, 424; Staphylococcus_aureus_HC (tooth infection, abscesses, heart disease, invades tumors)—18819.51, 936.97, 18968.87, 944.40; Staphylococcus_coagulae_positive—643; Staphylococcus_comp—40887, 9646, 8697, 7270, 7160, 2600, 2431, 1902, 1109, 1089, 1060, 1050, 1010, 999, 985, 960, 958, 943, 934, 884, 882, 880, 878, 876 824.4, 787, 786, 784, 745, 738, 728, 727, 718, 686, 678, 674, 647, 644, 643, 639, 634, 576, 563, 555, 550, 542, 478, 453, 436, 424, 423, 411, 333, 134, 128; Staphylococcus_general (728*, 786*)—7160, 1109, 1089, 885, 884, 883, 882, 881, 880, 879, 878, 877, 876, 875, 786, 728, 674, 639, 634, 550, 453; Stemonius_HC—10505.07, 523.02; Stemphylium—461, 340, 114; Sterigniatocystin_HC—6621.68, 6273.17, 4779.56, 4381.26, 329.67, 312.32, 237.96, 218.13; Stiff_muscles_comp—6000, 1800, 1550, 880, 802, 787, 776, 776, 766, 728, 727, 328, 320, 304, 300, 250, 240, 160, 125, 80, 40, 20, 10, 9.19, 6, 5.8, 4.9, 2.5, 1.5, 1.2, 1, 0.5; Stiff_muscles—320, 328, 304, 300, 240, 160, 776, 728, 1800, 125, 80, 40, 20, 6000; Stiff_muscles_secondary—250, 10, 5.8, 2,5, 11.5, 1.2, 1. 0.5, 1550, 802, 880, 787, 776; Stiff_neck—4.9, 6, 9.19; Stiff_shoulder (see also Rheumatism)—10000, 727, 766, 20; Stigeoclonium_HC—20512.27, 1021.25, 20263.34, 1008.85; Stomach disorders (see also E. Coli and Parasites general if no lasting relief)—21127, 2008, 1552, 880, 832, 802, 784, 727, 690, 676, 664, 450, 422, 125, 95, 72, 20, 3.9; Stomach_gas—50, 51, 52, 53, 56, 59, Stomatitis (Inflammation of mucous membranes in the mouth. See also Candida, Herpes simplex I, Stomatitis aphthous, Pyorrhea, and Gingivitis)—465, 677, 702, 787, 234, 278, 568, 672; Stomatitis_aphthous (ulcers on mucous membrane of mouth. Also called canker sores. All freqs for 5 min)—478, 487, 498, 788, 955, 982, 1902, 11904, 1906; Stomatitis_aphthous_1 (all freqs for 5 min)—1901, 1902, 1903, 1904, 1905, 1906, 1907; Stomatitis_aphthous_v—888, 880, 848, 846, 831, 685, 742, 734 , 1043, 944, 782, 591, 480, 423, 343, 339, 322, 832, 556, 808, 534, 460, 424, 246; Strep_and_Staph_v—9647, 40887, 7160, 2431, 1902, 1109, 1060, 1050, 1010, 985, 958, 935, 786, 727, 718, 686, 643, 576, 563, 542, 453, 436, 423, 411, 333, 134, 128; Strep_mutans (use Strep viridans); Strep_sweep_TR (sweep for strep pneumonia, group G, and pyogenes strains. Sweep from 5632.8152 to 5763 by 0.015625 and 5763 to 6053.125 by 0.03125 for dwell 0.25 and pulse 64 75); Strep_virus—563, 611, 727; Streptococcus_enterococcinum (can cause infection in the digestive and urinary tracts)—686, 409; Streptococcus_hemolytic (blood infection by strep, 1522*, 535*, 368*, 318*)—728, 880, 786, 712, 128, 134, 334, 443, 535, 542, 675, 1415, 1522, 1902, 691, 710, 1203, 368, 318; Streptococcus_infection_general (streptococcus family. Also see General antiseptic and other Strep sets, 880*)—2000, 1266, 885, 884, 883, 882, 881, 880, 879, 878, 877, 876, 875, 848, 802, 800, 787, 784, 727; Streptococcus_lactis_HC (occurs in milk)—19168.02, 954.32; Streptococcus_mitis_HC (lung infection, tooth infection, abscesses, stiff knees)—15832.29, 788.24; Streptococcus_mutant_strain—114, 437, 625, 883, 994; Streptococcus_mutant_strain_secondary—108, 433, 488, 687, 833, 8686, 8777, 9676, 660, 732, 745, 754, 764; Streptococcus_Pepto (can infect digestive tract)—201, 629; Streptococcus_pneumoniae (can cause pneumonia, empyema, middle car infections, endocarditis, peritonitis, arthritis, bacteremia, and meningitis, 683*, 688*)—231, 232, 683, 688, 776, 766, 728, 846, 1552, 8865; Streptococcus_pneumoniae_HC (pneumonia, inner ear disease)—18321.64, 912.18; Streptococcus_pneumoniae_hi_alt—1136.7, 2273.4, 46275, 46065.5, 46000, 45856, 23138, 23032.8, 23000, 22938, 4546.9, 18187.4, 36374.8, 72749.6, 625.5, 20015.3, 40030.6; Streptococcus_pneumoniae_mixed_flora—158, 174, 645, 801; Streptococcus_pyogenes (pus forming infections. Can cause sore throat, skin inflammation, scarlet fever, pharyngitis, scarlet fever, impetigo, erysipelas, cellulitis, septicemia, toxic shock syndrome, and acute glomerulonephritis See also General antiseptic)—10000, 5004, 8450, 2502, 2111, 1214, 880, 880, 845, 787, 776, 735, 727, 720, 660, 625.5, 616, 465, 20; Streptoccus_pyogenes_HC (infects teeth)—18570.58, 924.57; Streptococcus_sp_group_G_HC—18321.64, 912.18; Streptococcus_viridans (425*, 433*)—425, 433, 445, 935, 1010, 1060, 8478, 457, 465, 777, 778, 1214, 1216; Streptomyces_griseus (soil bacteria which yields streptomycin)—333, 887; Streptothrix (includes Actinomycosis, Nocardia, and Actinomyces israeli)—10000, 7880, 7870, 2890, 2154, 887, 787, 784, 747, 727, 678, 567, 488, 465, 262, 237, 231, 228, 222, 192, 157, 20; Stroke_follow_up (ensure adequate water, minerals especially magnesium, and omega 3 intake and consider using enzymes like bromelain to inhibit clotting)—2112, 3, 203, 1800, 880, 787, 727, 650, 625, 600, 125, 95, 72, 1865, 522, 428, 10000, 20, 2720; Strongyloides (use Parasites, strongyloides); Strum (family of organisms that can infect the thyroid causing goiter. Use kelp internally. Use Struma cystica, nodosa, and parenchyme freqs.); Struma_cystica—5311, 531, 756, 361; Struma_nodosa—105, 122, 321, 517, 532, 651, 714; Struma_parenchyme—121, 576; Stye (staph infection of sebaceous gland of eyelash. Also use Staphylococci infection set)—10000, 880, 787, 727, 20, 453, 2600; Subluxation_induced_disorders—9.6; Sudor_pedis (excessive foot sweat)—148; Sun_allergy (Examine prescription drugs, such as Psoralen, for photo sensitization properties.)—3, 330, 10000; Sunstroke—444, 440, 190, 3000, 95, 522, 146, 880, 20, 10000; Surgery_anaesthesia_detox (see also Liver support)—522, 146; Surgery_preop_postop_prevent_infections (see also General antiseptic, Staph, and Strep sets)—2170, 1800, 1600, 1550, 802, 1500, 880, 832, 802, 787, 776, 727, 465, 444, 522, 146, 428; Surgical_pain_post_op—3000, 2720, 95; SV40 (use Simian_virus_40); Swelling (edema. See also Kidney insufficiency and Lymph stasis freqs)—522, 146, 6.3, 148, 444, 440, 880 787, 727, 20, 10000, 5000, 3000; Swimmers_ear (also see Pseudomonas aeruginosa and Otitis externa, To help prevent, always disinfect and dry ears after using public pools with 40 percent alcohol solution such as vodka or white rum)—728, 884, 880, 464, 174, 482, 5311; Swollen_glands (use Lymph and see Mumps freq sets as appropriate); Syphilis—6600, 789, 900, 2776, 177, 650, 625, 600, 660, 658;

T-lymph_virus_TR—8225, 6353, 6230, 3806, 2664, 2432, 1675, 1230, 1220, 965, 844, 837, 776, 732, 725, 714, 646, 63:3, 392, 314, 245, 243, 111; Tachycardia (rapid heart beat. For lab animals only. See also Heart tonic, Relaxation sets)—1 Taenia (use Parasites tapeworm); Tainio_sweep (sweep from 42 to 72 Hz in small intervals, at most 1, to possibly stimulate the immune system); Taste_lack_of—10000, 20; Tendomyopathy—320, 250, 160, 80, 40, 20, 10, 5.8, 2,5, 1.5, 1.2, 1, 0.5; Tetanus (infectious disease of the central nervous system caused by clostridium tetani)—15770, 1200, 1142, 880, 700, 628, 600, 554, 465, 458, 400, 36:3, :352, 244, 234, 120, 20; Tetanus_secondary—880, 787, 727: Tetragenus—393, 43:3, 2712; Thalamus_stimulant—20; Thermi_bacteria—233, 441; Throat_tickle_chronic (see also Streptococcus and Strep pneumonia sets)—120, 666, 690, 727, 787, 800, 880, 1560, 1840, 1998, 766, 776; Thrombophlebitis (inflammation of vein walls from clotting. Use on lab animals only, See also Circulatory stasis and Stroke_followup)—1500, 776, 685; Thrombosis_infective_herpes_type (not to be used with arrhythmia or in U.S.)—2720, 2489, 2170, 1800, 1550, 802, 880, 787, 727, 444, 125, 95, 72, 20, 444, 1865, 1489; Thrush (use Candida and see Stomatitis freqs)—414, 465; Thymus_stimulation—20; Tinea cruris (use Epidermophyton floccinum); Tinea_pedis (use Trichophyon mentagrophyte); Tinea_versicolor (use Malassezia furfur); Tinnitus (see also Circulatory stasis, Dental, Dental foci, General antiseptic, and Otitis freqs)—20, 2720, 728, 784, 880; Tobacco_nosaic—233, 274, 543, 782, 1052; Tobacco_mosaic_virus_HC—21308.87, 1060.91; Tonsillar_nodules—246, 151, 414; Tonsillar_nosode—1656; Tonsillitis (inflammation of tonsils)—1.2, 73, 1550, 802, 1500, 880, 832, 787, 776, 727, 650, 625, 600, 465, 144, 452, 582; Tooth_extraction_followup (see also General antiseptic set)—3000, 2720, 95, 47.5, 7.82; Toothache (to neglect this can prevent recovery from any illness. Should also be treated professionally. See also Dental. Dental foci, Gingivitis, Pyorrhea.)—5170, 3000, 2720, 2489, 1800, 1600, 1550, 1500, 880, 832, 802, 787, 776, 776, 727, 666, 650, 646, 600, 465, 190, 95, 47.5; Toxin_elimination—0.5, 522, 146, 1552, 800; Toxoplasma_HC (human strain)—19665.89, 979.11; Toxoplasmosis (a serious, infectious disease that can be either acquired or present at birth and that is commonly contracted by handling contaminated cat litter)—434, 852; Trachoma (use Chlamydia trachomatis); Transformation_series (includes 18 ZOBET, 6 of which are the Sacred Solfeggio, 9 Activation, One KI, and the Sacred Spiral)—9999, 147, 174, 417, 471, 714, 741, 258, 285, 528, 582, 825, 852, 369, 396, 639, 693, 936, 963, 2664, 3330, 3996, 774, 855, 1584, 1746, 2475, 2556, 9990, 144, 234, 378, 612, 991, 1604; Trauma—96, 192, 300, 760, 3000; Trench_mouth (use Gingivitis); Treponema_pallidum (use also Syphilis)—660, 902; Treponema_pallidum_HC (causes syphilis)—17276.11, 860.13; Trichinosis (use Parasites_trichinosis); Trichodermia—711; Trichomonas nicroorganism causing vaginal irritation with discharge and itching)—610, 692, 980; Trichophyton_general—132, 725, 808, 812, 2422, 9493, Trichophylon_mentagrophytes, Trichophyton_nagel—381, 585, 593. 812; Trichophyton_nagel_secondary—133, 142, 373, 376, 378, 385, 387, 420, 425, 428, 576, 578, 580, 581, 583, 584, 587, 588, 592, 595, 597, 724, 725, 726, 750, 794, 797, 801, 805, 808, 809, 817, 886, 2422, 6887, 7688, 7697, 7885, 584, 587, 592, 732, 733, 738, 748, 765, 766, 771, 777, 778, 779, 1256; Trichophyton_rubnum—752, 923, Trichophyton_tonsuraus—454, 765; Trigeminal_neuralgia—2720, 2489, 2170, 1800, 1600, 1550, 802, 7.5, 880, 832, 787, 776, 760, 727, 650, 146, 7,82, 27.5, 428; Troglodytella_abrassari_HC—19068.45, 949,36, 20860.78, 1038.60; Trypanosoma_gambiense—255, 316, 403, 700, 724; Tuherculinum—332, 522, 664, 731, 737, 748, 1085, 1099, 1700, 761, Tuberculosis (also see Tuberculinum. Some think some chronic lung infections can actually be a mild case of TB and recommend to run a TB set periodically, especially after traveling on airplanes and/or abroad, 11552*, 802*)—8030, 16000, 1840, 1600, 1552, 1550, 1513, 1500, 803, 802, 784, 740, 727, 720, 690, 666, 583, 541, 369, 216, 20; Tuberculosis_aviare—303, 332, 342, 438, 440, 532, 3113, 6515, 697, 698, 720, 731, 741, 748, 770: Tuberculosis_bovine—229, 523, 625, 635, 838, 877, 3353, 748, 757; Tuberculosis_klebsiella—217, 220, 221, 686, 1132, 1644, 2313, 6516, 729, 748; Tuberculosis_rod_E_colikinfections—799, 802, 804, 1550, 1513; Tuberculosis_rod_form (803*)—8030, 369, 583, 541, 802, 803, 1513; Tuberculosis_secondary_complications—776, 2127, 2008, 465; Tuberculosis_virus (1552*)—2565, 1552; Tularemia (a serious infectious disease also called deerfly fever or rabbit fever)—324, 427, 823; Tumor_brain (use Cancer, astrocytoma, glioma, glioblastoma); Tumor_benign—1, 10, 10.2, 10,4, 10.6, 10.8, 11; Turbatrix—104; Typhoid_fever (typhus infection related symptoms causing high fever, headache, and rash. Also use Salmonella typhi and Rickettsia and see Q fever,)—9680, 18620, 13944, 6900, 3205, 1868, 1866, 1864, 1862, 1860, 1800, 1550, 1445, 900, 869, 824, 802, 760, 714, 712, 690;

Ulcer_duodenal—676, 727, 750, 880, 10000; Ulcer_gastric—676; Ulcer_parasites—3, 3.5, 4, 6,8, 9.9, Ulcer_ventric—142, 566, 676, 232, 1000; Ulcer_ventricular—769, 760; Ulcers_general—676, 664, 802, 784, 2489, 2170, 2127, 1800, 1600, 880, 832, 802, 787, 776, 727, 73; Urea_plasma—756; Uremia (also known as uremic poisoning; excessive amounts of nitrogenous waste products in the blood, as seen in kidney failure. Also use Kidney and Lymph stasis freqs.)—911; Urethritis (also see Vaginosis and use Chlamydia trachomatis)—2720, 2170, 2127, 1800, 1600, 1550, 802, 1500, 880, 832, 787, 776, 727, 660, 650, 625, 600, 465, 444, 1865, 125, 95, 72, 1.2; Urinary_Tract_Infections (also see Bacterium coli sets)—2050, 880, 1550, 802, 787, 727, 465, 20, 9.39, 642, 358, 539; Urticaria (hives, often due to toxins)—1800, 880, 787, 727, 522, 146, 4.9; Uterine_polyp—689;

Vaccinium (a homeopathic nosode)—476; Vaginosis (non-specified infection. Also see Gardinerella, Candida, and Trichomonas)—414, 542, 642, 652, 800, 832, 845, 866, 942, 728, 784, 880, 464; Varicella (use herpes zoster); Varicoses—1.2, 28; Variola (use Smallpox); Vegetative_dystonia (involuntary muscle dysfunction)—40; Veillonella_dispar_HC—20064.19, 998.94; Vein_thrombosis (use Thrombophlebitis); Vemica (use Wart_vernica); Vertigo (also use Otitis, Meniere's, and see General antiseptic freqs)—60, 5.8, 4; Vertigo_TR—880, 802, 784, 787, 786, 766, 522, 727, 720, 688, 683, 652, 645, 316, 72, 92, 60, 58, 40, 1550; Viral_complex_TR—10000, 7344, 5000, 2950, 2900, 2650, 2600, 1550, 1234, 430, 620, 624, 646, 866, 5148, 2213, 1918, 742.4, 303, 23.2, 20, 864, 790, 690, 610, 470, 484, 986, 644, 254, 30, 33, 6000, 599, 611, 613, 2127, 2080, 2050, 2013, 2008, 2003, 2000, 1850, 880, 803, 800, 787, 727, 660, 484, 465, 440, 35, 500, 200, 68; Vision poor (see also Cataracts. Run 1830 for 15 minutes.)—350, 360, 1830; Visual_acuity (run 1830 for 10 minutes)—3176, 1860, 1856, 1852, 1848, 1846, 1842, 1838, 1834, 1830, 1826, 1822, 1818, 1814, 1810, 1806, 1802, 360, 350; Vitamag_complete_set—1, 2, 3, 4, 5, 6, 7, 7.8, 9, 10, 13, 16, 19.5, 22.5, 24; Vitiligo (loss of pigmentation in areas of skin. Use with B complex with extra PABA internally and PABA cream topically. Also see E. coli, Parasite General, Liver support, and Fungus freqs if necessary)—440, 600, 650, 2112, 880, 787, 727, 444, 20;

Wart_condyloma (venereal warts caused by infectious papilloma virus)—466, 907; Warts_general (also see Papilloma and Parasites roundworms and Parasites, flukes general)—2720, 2489, 2170, 2127, 2008, 1800, 1600, 1500, 907, 915, 874, 727, 690, 666, 660, 644, 767, 953, 495, 466, 110; Warts_1 (apply with pad at negative 24v for 5 min at a time)—21750; Warts_papilloma (branch or stalk, use Papilloma virus); Warts_plantar (use 915 and 918 for 15 to 30 min)—915, 918, 20, 120, 727.5, 787, 880, 2008, 2127.5; Warts_verruca (a rough surfaced, supposedly harmless, viral caused skin wart)—495, 644, 767, 797, 877, 953, 173, 787; Wellness—6.8, 7.83; Werlhof—690, 452; West_Nile_1—413, 826, 1239, 3303, 465, 841, 8410; West_Nile_2—1385, 1115, 841, 799, 730, 697, 686, 664, 514, 498, 484, 434, 404, 313, 240; Wheat_smut—10163, 156, 375; Wheat_stem_rust1—643; Whiplash—20, 2720, 10000; White Blood Cell stimulation (use Immune system stimulation); Whooping_cough (use Pertussis); Wolhynia_fever (a Rickettsia illness, transmitted by lice, that is debilitating and conducive to relapse. Also see Rickettsia)—547; Wound_healing (also see General antiseptic)—2720, 880, 787, 727, 220, 190, 20, 40;

Yeast_cervical (also use Candida)—788, 706, 771; Yeast_general (also see Candida. Use Parasites general, roundworm, and ascaris if no lasting relief)—72, 254, 375, 522, 876, 987, 414, 422, 465, 582, 787, 1016, 2222; Yeast_general_v—72, 422, 582, 787, 1016, 2222, 1134, 254, 987, 986, 984, 982, 980, 974, 908, 906, 878, 876, 873, 871, 866, 865, 864, 863, 861, 778; Yellow_fever (a severe viral infection which can cause damage to the liver, kidneys, heart, and entire gastrointestinal tract)—20, 142, 178, 232, 432, 734, 1187, 733, 779, 10000; Yellow_fly—996; Yerseni_pestis (aka Bubonic plague; spread primarily by fleas on rats)—5120, 5000, 1600, 512, 508, 504, 500, 496, 492, 338, 333, 210, 216, 160; Yersenia_pestis_1—337.6; and

Zearalenone_HC—4978.71 247.88; Zygomycosis (also called mucormycosis; a serious fungal infection usually^{,} associated with uncontrolled diabetes mellitus or immunosuppressive drugs)—942, 623, 73.

### POTENTIAL ASPECTS OR ELEMENTS OF THE CLAIMED INVENTION THAT CAN BE OPTIONALLY EXCLUDED OR NEGATIVELY CLAIMED

The present invention can also in particular claimed embodiments exclude or negatively claim one or more aspects, e.g., to more particularly recite or exclude embodiments or elements that might occur in cited or other published art, as presented herein. Accordingly, the present invention can optionally exclude, not include, or not provide, one of more, or any combination of any element, feature, component or step disclosed herein.

A number of implementations have been described. Nevertheless, it can be understood. that various modifications may be made. For example, elements of one or more implementations may be combined, deleted, modified, or supplemented to form further implementations. As yet another example, the logic flows depicted in the figures do not require the particular order shown, or sequential order, to achieve desirable results, In addition, other steps may be provided, or steps may be eliminated, from the described flows, and other components may be added to, or removed from, the described systems. Accordingly, other implementations are within the scope of the following claims.

What is claimed is:

1. A method for improving properties or functions of cells, tissues, proteins, fats, or organs in vitro, in vivo,or in situ, by treating the cells, tissues, proteins, fats, or organs with one or more pulsed electromagnetic frequencies (EMFs), the method comprising:
treating the cetissues,lls, proteins, fats, or organs with pulsed EMFs comprising more of frequencies selected from 963, 852, 741, 639, 528, 417, 396, 288, and 174, or resonant frequencies, harmonics, or sub harmonics thereof, including pulses of EMFs including sound and/or laser light comprising two or more of frequencies between 0.25 Hz and 11780000 Hz;
wherein the EMF pulses range from tens of nanoseconds to ten femtoseconds and have durations selected from 100 ps to 10,000 nanoseconds.

2. A method of claim 1. wherein the improved properties or function of cells, tissues, proteins, fats, or organs is due to removal or inactivation of one or more of a non-naturally occurring substance selected from at least one of an impurity, a contaminant, an inorganic chemical, a pharmaceutical or a metabolite thereof, a toxin, a carcinogen, a pathogen, a pollutant, a metal, a salt, a sugar, or ionic form thereof, a radioactive material or radioactive particle, or a microbiotic.

3. A method of claim 2, wherein the one or more of a non-naturally occurring substance is in the form of one or more of a molecule, a compound, a composition, a mixture, a suspension, an emulsion, a dispersion, an ion, a salt, a sugar, a microparticle, a nanoparticle, or treated inflammation of cells, tissues, proteins, fats, or organs in vitro, in vivo, or in situ.

4. A method of claim 2, wherein the one or more of a purified natural, or a non-naturally occurring, substance selected from a chemical, a toxin, a metal, a microorganism, a prescription drug, a food contaminant, a pesticide, an herbicide, a hormone analog, a neurotransmitter analog, and a water contaminant.

5. A method of claim 1, wherein the method further comprises treating the cells, tissues, proteins, fats, or organs the EMF treated cells, tissues, proteins, fats, or organs or the EMF treated cells, tissues, proteins, fats, or organs with one or more of ultraviolet exposure to provide UV, EMF treated cells, tissues, proteins, fats, or organs in vitro, in vivo, or in situ.

6. A method of claim 1, wherein the application step (a) and/or the applying step (b) comprises the use of laser generating pulses of light, pulsed sound frequencies and pulsed electromagnetic fields (EMFs) at pulsed frequencies using a frequency tone generator or system of frequency tone generators using one or more copper rings to produce the one or more EMF frequencies.

7. A method of claim 6, wherein the use of laser generating pulses of light, pulsed sound frequencies and pulsed electromagnetic fields (EMFs) at pulsed frequencies using a frequency tone generator or system of frequency tone generators using one or more copper rings are provided as one or more copper rings with substantially the same radius, wherein the one or more copper rings intersect with each other such that the center of each disk is adjacent to a perimeter portion of the other copper rings.

8. A method of claim 6, wherein the use of laser generating pulses of light, pulsed sound frequencies and pulsed electromagnetic fields (EMFs) at pulsed frequencies using a frequency tone generator or system of frequency tone generators using one or more copper rings vibrate at said at least one or more pulsed electromagnetic frequencies corresponding to the length, or a fraction or multiple of the length, of at least a portion of the copper rings.

9. A method of claim 6, wherein the use of laser generating pulses of light, pulsed sound frequencies and pulsed electromagnetic fields (EMFs) at pulsed frequencies using a frequency tone generator or system of frequency tone generators wherein a primary coil of the copper rings at said one or more EMF frequencies that are resonant with a secondary frequency at which a secondary coil of the copper rings vibrate that balances the force of gravity within a magnetic field produced by the copper rings.

10. A method of claim 1, wherein the EMF treated cells, tissues, proteins, fats, or organs substantially comprise mammalian cells, tissues, proteins, fats, or organs in vitro, in vivo, or in situ.

11. A method of claim 1, wherein the EMF treated cells, tissues, proteins, fats, or organs are selected from the blood, adrenals, kidney, liver, bladder, intestines, heart, diaphragm, spleen, lungs, colon, gall bladder, pancreas, stomach, brain, fat cells, muscles or bone for improving properties or fiinctions of cells, tissues, proteins, fats, or organs in vitro, in vivo, or in situ.

12. A method of claim 1, wherein the improved properties or functions of cells, tissues, proteins, fats, or organs include one or more of the increase, reduction, or improvement of one or more of: carbon dioxide (CO2), oxygen (O2), pH, carbonate, hiocarhonate, inflammation, magnesium, calcium, phosphate, potassium, chlorine, chloride, manganese, selenium, aluminum, mercury, monatomic gold, atomic geometry, amino acid(s), protein, saturated fatty acids, unsaturated fatty acids, sugars, triglycerides, ATPs, AMPS, ADPs, nucleic acids, magnetic field, electric field, electromagnetic field, or the presence of non-naturally occurring compounds or compositions, mutations in cells, tissues, proteins, fats, or organs of diseased cells, tissues, proteins, fats, or organs of DNA damaged cells, tissues, proteins, fats, or organs of abnormal cells, mutation cells, cancer cells, pathogens; and cellular markers of aging;
wherein the increase, reduction, or improvement is to an amount or concentration closer to normal physiological levels.

13. A method of claim 1, wherein the EMF treated cells, tissues, proteins, fats, or organs are selected wherein the increase, reduction, or improvement of the sugar levels in the blood is to an amount or concentration closer to normal physiological levels in the blood.
